(19) United States
(12) Patent Application Publication
(10) Pub. No.: US 2026/0158171 A1
(43) Pub. Date: Jun. 11, 2026

(54) GENE THERAPIES FOR METABOLIC DISORDERS

(71) Applicant: Fractyl Health, Inc., Lexington, MA (US)

(72) Inventors: Jason West, Lexington, MA (US); Jay Caplan, Lexington, MA (US)

(73) Assignee: Fractyl Health, Inc., Lexington, MA (US)

(21) Appl. No.: 18/720,429

(22) PCT Filed: Dec. 14, 2022
(86) PCT No.: PCT/US2022/081543

§ 371 Date: Jun. 14, 2024

Related U.S. Application Data
(60) Provisional application No. 63/289,850, filed on Dec. 15, 2021.
(60) Provisional application No. 63/423,411, filed on Nov. 7, 2022.

(51) Int. Cl. A61K 48/00; A61K 38/26; A61P 3/10; C12N 15/86

(52) U.S. Cl. CPC A61K 48/0058; A61K 38/26; A61K 48/0075; A61K 48/0083; A61P 3/10; C12N 15/86; C12N 2750/14143

(57) ABSTRACT

The disclosure provides insulin-glucagon-like peptide 1 (GLP-1) polynucleotides for delivery to pancreatic islet cells, as well as compositions comprising the polynucleotides and methods of treating obesity-associated metabolic disorders.

### RELATED APPLICATIONS

This application claims the benefit under 35 U.S.C. § 119 of U.S. provisional application 63/289,850, filed Dec. 15, 2021, and U.S. provisional application 63/423,411, filed Nov. 7, 2022, the entire contents of each of which are incorporated herein by reference.

### REFERENCE TO AN ELECTRONIC SEQUENCE LISTING

The contents of the electronic sequence listing (F085770000WO00-SEQ-HJD.xml; Size: 103,314 bytes; and Date of Creation: Dec. 13, 2022) is herein incorporated by reference in its entirety.

### BACKGROUND

Metabolic diseases, such as type 2 diabetes (T2D) account for significant morbidity and mortality globally. Approximately 50% of the estimated 27 million people diagnosed with T2D in the United States have inadequately controlled disease despite the availability of over 60 approved drugs, and an estimated 50 million people are expected to be living with T2D in the United States by 2035. Most of the currently available therapies aim to treat metabolic diseases by managing the symptoms, rather than treating the underlying causes of the disease.

### SUMMARY

A primary role of pancreatic beta cells is to produce and secrete endogenous insulin in response to nutrients. Insulin production and secretion from a beta cell is a multi-step, complex process that includes significant post-translational processing and trafficking of the insulin gene product in order to load functional insulin hormone peptides into nutrient-responsive secretory vesicles of the beta cell (see FIG. 1). These vesicles fuse with the plasma membrane to release functional insulin in response to nutrients, particularly glucose.

Glucoregulatory hormones, including but not limited to GLP-1, are often produced and secreted in response to dietary nutrients and impact pancreatic beta cell function in a multitude of ways. Primary among their effects on beta cells include stimulation of insulin secretion and production, as well as positively impacting beta cell health. Beta cell health improvements reported for GLP-1 include the maintenance of beta cell mass through increased cell proliferation, beta cell neogenesis, and/or the inhibition of apoptosis. Applying these beneficial functions of glucoregulatory hormones for the treatment of diabetes and related disorders has been a successful clinical strategy and remains an active area of therapeutic research.

Many glucoregulatory hormones possess two properties that make their pharmacologic application to disease treatment challenging: (1) a short half-life, and (2) side effects of nausea and vomiting when present in circulation at chronically at high levels. To overcome these challenges, the present disclosure provides, in some aspects, methods to produce transgenic glucoregulatory hormones in a sustained way and in response to nutrients to mimic their endogenous production. This manner of production limits sustained high levels in circulation. To achieve this, the technology described herein leverages pancreatic beta cells for the production and secretion of transgenic glucoregulatory hormones, as these cells already perform a similar function for endogenous insulin production and secretion. Furthermore, local production of glucoregulatory hormones by the pancreatic beta cell produce these hormones at one of their primary sites of action, thereby reduce the circulating levels of hormone needed to achieve a desired effect on beta cell function.

The methods of the present disclosure provide a way to encode transgenic glucoregulatory hormones that are functionally active and loaded into nutrient-responsive secretory vesicles akin to endogenous insulin. By fusing the glucoregulatory hormone (payload) into a copy of a transgenic insulin sequence enables post-translational processing, secretory-vesicle loading, and nutrient-responsive payload secretion by beta cells (see FIG. 2). This payload needs to be processed into a functional and independent peptide. Thus, the constructs provided herein are strategically designed with respect to both the position where the in-frame insertion with the transgenic Insulin sequence occurs as well as the flanking sequences that facilitate processing of the transgenic glucoregulatory hormone into a functional peptide that is independent from the transgenic insulin peptides.

Some aspects provide an endoscopic gene therapy method, comprising: advancing a depositing device comprising at least one depositing element to at least one pancreatic deposit site in a patient having a metabolic disease and/or a pancreatic disease; and delivering an effective amount of a treatment agent through the at least one depositing element into the at least one pancreatic deposit site, wherein the treatment agent comprises an adeno-associated virus (AAV) vector that comprises an AAV vector genome, wherein the AAV vector genome comprises a polynucleotide comprising a human pancreatic islet beta cell-specific promoter operably linked to (a) a human GLP-1 receptor agonist coding sequence, optionally a human GLP-1 coding sequence, or (b) a peptide tyrosine tyrosine (PYY) coding sequence.

In some embodiments, the at least one depositing element comprises at least one needle positioned on a distal portion of the depositing device.

In some embodiments, the distal end of the depositing device is delivered into the patient through the mouth, and advanced through a wall of the gastrointestinal tract to a location proximate the pancreas, optionally wherein (a) the depositing device is delivered through a working channel of a gastrointestinal endoscope that has been delivered through the mouth of the patient of (b) the depositing device is delivered alongside a gastrointestinal endoscope that has been delivered through the mouth of the patient.

In some embodiments: the metabolic disease is selected from the group consisting of: Type 1 diabetes; Type 2 diabetes; nonalcoholic fatty liver disease (NAFLD); nonalcoholic steatohepatitis (NASH); obesity; and combinations thereof; or the pancreatic disease is selected from the group consisting of: pancreatitis; pancreatic cancer; hyperinsulinism; and combinations thereof.

In some embodiments, the at least one pancreatic deposit site is selected from the group consisting of: intraparenchymal space; anterior pararenal space; intraductal space; intraarterial space of an artery that feeds at least a portion of the pancreas; and combinations thereof, preferably wherein the at least one pancreatic deposit site is the intraparenchymal space.

In some embodiments, the delivering of a treatment agent comprises at least a first delivery in which a minimum volume of the treatment agent is delivered into the pancreatic parenchyma, and wherein the minimum volume of treatment agent comprises a volume sufficient to cause at least a portion of the volume of the treatment agent to exit into the anterior pararenal space, spread, and re-enter the pancreas, optionally wherein the method further comprises at least a second delivery of the treatment agent to at least one additional deposit site proximate the tail of the pancreas.

In some embodiments, the delivering of a treatment agent comprises at least a first delivery in which a minimum volume of treatment agent is delivered into the pancreatic parenchyma, and wherein the minimum volume of the treatment agent comprises a volume of at least 2 ml, at least 3 ml, and/or at least 5 ml.

In some embodiments, the depositing device is advanced to the at least one pancreatic deposit site under image guidance, optionally, wherein the image guidance comprises: endoscopic ultrasound guidance; computerized tomography (CT) guidance; and/or magnetic Resonance Imaging (MRI) guidance.

In some embodiments, the at least one pancreatic deposit site comprises locations within 10 cm, 7.5 cm, 5 cm, and/or 3 cm of a portion of the pancreas, and wherein the portion of the pancreas comprises the tail, the neck, the body, the head, and/or the uncinate process.

In some embodiments, the treatment agent and/or the at least one depositing element is configured to be visualized by an imaging device, and wherein the method further comprises visualizing the treatment agent and/or the at least one depositing element with the imaging device to confirm proper delivery of the treatment agent.

In some embodiments, the method further comprises delivering an imaging agent through the at least one depositing element and visualizing the delivery of the imaging agent with an imaging device to confirm subsequently proper delivery of the treatment agent.

In some embodiments, the method further comprises pre-loading the depositing device with the treatment agent, optionally wherein the treatment agent is loaded into the depositing device from the distal end of the depositing device.

In some embodiments, the delivering of a treatment agent is performed at a pressure of at least 3 mmHg and/or no more than 25 mmHg.

In some embodiments, the delivering of a treatment agent is performed at a flow rate of at least 1 ml/min and/or no more than 5 ml/min.

In some embodiments, the at least one depositing element comprises multiple fenestrations along its length.

In some embodiments, the method further comprises confirming the at least one depositing element is in a proper location prior to delivering the treatment agent.

In some embodiments, the method further comprises delivering a permeability-enhancing agent prior to the delivering of the treatment agent and/or simultaneously with the delivering of the treatment agent, optionally wherein the delivering of the permeability-enhancing agent is performed locally and/or intravenously, optionally wherein the permeability-enhancing agent comprises an agent selected from the group consisting of: hyaluronidase; collagenase; losartan; and combinations thereof, and optionally wherein the treatment agent comprises a coformulation of the treatment agent and the permeability-enhancing agent.

In some embodiments, the method further comprises heating tissue proximate the at least one pancreatic deposit site to a temperature above 39° C. prior to, during, and/or after the delivery of the treatment agent.

In some embodiments, the method further comprises delivering a dissemination-blocking material that is configured to prevent undesired dissemination of the treatment agent to non-target locations, optionally wherein the dissemination-blocking material comprises a viscous substance and/or a polymer.

In some embodiments, the method further comprises positioning a blocking element in the patient, wherein the blocking element is configured to prevent undesired dissemination of the treatment agent to non-target locations.

In some embodiments, the method further comprises removing at least a portion of the treatment agent from a deposit site location after the delivery of the treatment agent begins.

In some embodiments, the method further comprises removing all of the treatment agent.

Some aspects relate to polynucleotide comprising a glucoregulatory hormone coding sequence and an insulin coding sequence.

In some embodiments, the glucoregulatory hormone coding sequence and the insulin coding sequence are arranged such that a functional glucoregulatory hormone and a functional insulin are produced in vivo following administration to a subject.

In some embodiments, the glucoregulatory hormone coding sequence is nested in the insulin coding sequence.

In some embodiments, the insulin coding sequence comprises, 5′ to 3′, a signal peptide coding sequence, a B-chain coding sequence, a C-peptide coding sequence, and an A-chain coding sequence, optionally wherein: (a) the glucoregulatory hormone coding sequence is located between the signal peptide coding sequence and the B-chain coding sequence; (b) the glucoregulatory hormone coding sequence is nested in the B-chain coding sequence; (c) the glucoregulatory hormone coding sequence is located between the B-chain coding sequence and the C-peptide coding sequence; (d) the glucoregulatory hormone coding sequence is nested in the C-peptide coding sequence; (e) glucoregulatory hormone coding sequence is located between the C-peptide coding sequence and the A-chain coding sequence; (f) the glucoregulatory hormone coding sequence is nested in the A-chain coding sequence; or (g) the glucoregulatory hormone coding sequence is located downstream from the A-chain coding sequence.

In some embodiments, (a) the glucoregulatory hormone coding sequence is flanked by a first PCSK1 (PC1/3) and/or PCSK2 (PC2) enzyme processing sequence, optionally a native PCSK1 or PCSK2 enzyme processing sequence or an artificial PCSK1 or PCSK2 enzyme processing sequence; or (b) the glucoregulatory hormone coding sequence is flanked by a first PCSK1 and/or PCSK2 enzyme processing sequence and a second PCSK1 and/or PCSK2 enzyme processing sequence, optionally a native PCSK1 or PCSK2 enzyme processing sequence or an artificial PCSK1 or PCSK2 enzyme processing sequence.

In some embodiments, the polynucleotide comprises, 5′ to 3′: (a) a signal peptide coding sequence, a glucoregulatory hormone coding sequence, a B-chain coding sequence, a first PCSK1 or PCSK2 enzyme processing sequence, a C-peptide coding sequence, a second PCSK1 or PCSK2 enzyme processing sequence, and an A-chain coding sequence; (b) a signal peptide coding sequence, a B-chain coding sequence, a glucoregulatory hormone coding sequence, a first PCSK1 or PCSK2 enzyme processing sequence, a C-peptide coding sequence, a second PCSK1 or PCSK2 enzyme processing sequence, and an A-chain coding sequence; (c) a signal peptide coding sequence, a B-chain coding sequence, a first PCSK1 or PCSK2 enzyme processing sequence, a glucoregulatory hormone coding sequence, a C-peptide coding sequence, a second PCSK1 or PCSK2 enzyme processing sequence, and an A-chain coding sequence; (d) a signal peptide coding sequence, a B-chain coding sequence, a first PCSK1 or PCSK2 enzyme processing sequence, a glucoregulatory hormone coding sequence nested in a C-peptide coding sequence, a second PCSK1 or PCSK2 enzyme processing sequence, and an A-chain coding sequence; (e) a signal peptide coding sequence, a B-chain coding sequence, a first PCSK1 or PCSK2 enzyme processing sequence, a C-peptide coding sequence, a glucoregulatory hormone coding sequence, a second PCSK1 or PCSK2 enzyme processing sequence, and an A-chain coding sequence; (f) a signal peptide coding sequence, a B-chain coding sequence, a first PCSK1 or PCSK2 enzyme processing sequence, a C-peptide coding sequence, a second PCSK1 or PCSK2 enzyme processing sequence, a glucoregulatory hormone coding sequence, and an A-chain coding sequence; or (g) a signal peptide coding sequence, a B-chain coding sequence, a first PCSK1 or PCSK2 enzyme processing sequence, a C-peptide coding sequence, a second PCSK1 or PCSK2 enzyme processing sequence, an A-chain coding sequence, and a glucoregulatory hormone coding sequence.

In some embodiments, the glucoregulatory hormone is selected from glucagon, GLP-1, oxyntomodulin, glicentin, glicentin-related polypeptide (GRPP), major proglucagon fragment, intervening peptide 1 (IP-1), intervening peptide 2 (IP-2), GLP-2, glucose-dependent insulinotropic peptide (GIP), peptide tyrosine tyrosine (PYY), cholecystokinin (CCK), somatostatin, oxyntomodulin, ghrelin, amylin, glucagon, leptin, follistatin, insulin-like growth factor 1 (IGF1), vasoactive intestinal peptide (VIP), and growth hormone 1 (GH1), and peptides, variants and fusions thereof, optionally any one of SEQ ID NOs: 31-58, 93, and 94.

In some embodiments, the glucoregulatory hormone is a wild-type human GLP-1.

In some embodiments, the GLP-1 is a variant human GLP-1, optionally comprising a Gly8 substitution, relative to a wild-type human GLP-1.

In some embodiments, the polynucleotide comprises a nucleotide sequence having at least 80%, at least 85%, at least 90%, at least 95%, or 100% identity to the nucleotide sequence of any one of SEQ ID NOs: 59-71.

In some embodiments, the polynucleotide comprises a nucleotide sequence encoding a polypeptide comprising an amino acid sequence having at least 80%, at least 85%, at least 90%, at least 95%, or 100% identity to the amino acid sequence of any one of SEQ ID NOs: 72-84.

In some embodiments, the glucoregulatory hormone is a wild-type human PYY.

In some embodiments, the polynucleotide comprises a nucleotide sequence having at least 80%, at least 85%, at least 90%, at least 95%, or 100% identity to the nucleotide sequence of any one of SEQ ID NOs: 85-87.

In some embodiments, the polynucleotide comprises a nucleotide sequence encoding a polypeptide comprising an amino acid sequence having at least 80%, at least 85%, at least 90%, at least 95%, or 100% identity to the amino acid sequence of any one of SEQ ID NOs: 88-90.

In some embodiments, wherein the polynucleotide is operably linked to a promoter sequence, preferably a pancreatic islet cell promoter sequence, more preferably a pancreatic islet beta cell promoter sequence, optionally selected from: the human Insulin promoter, the mouse Insulin2 promoter, the mouse Insulin1 promoter, the rat Insulin2 promoter, the rat Insulin1 promoter, Slc2a, IAPP, NKX6.1, DLK1, MafA, Slc30a8/Znt8, PCSK1, and ADCYAP1.

In some embodiments, the polynucleotide further comprises an enhancer sequence.

Some aspects relate to vector comprising the polynucleotide of any one of the preceding paragraphs.

In some embodiments, the vector is a nonviral vector, optionally a plasmid, bacterial artificial chromosome, yeast artificial chromosome, or minicircle.

In some embodiments, the vector is a viral vector, optionally selected from selected from a retroviral vector, an adenovirus vector, a Herpes simplex virus (HSV) vector, and an adeno-associated virus (AAV) vector.

Other aspects relate to a recombinant adeno-associated virus (AAV) vector genome comprising the polynucleotide of any one of the preceding paragraphs.

In some embodiments, the recombinant AAV vector genome further comprises inverted terminal repeat (ITR) sequences, optionally flanking the polynucleotide, further optionally wherein the ITR sequences are selected from AAV1, AAV2, AAV3, AAV4, AAV5, AAV6, AAV7, AAV8, AAV9, AAV10 (AAVrh10), and AAV11 ITR sequences.

Further aspects relate to recombinant adeno-associated virus (AAV) vector comprising (a) the recombinant AAV vector genome of any one of the preceding paragraphs and (b) a capsid protein, optionally wherein the capsid protein is selected from AAV1, AAV2, AAV3, AAV4, AAV5, AAV6, AAV7, AAV8, AAV9, AAV10, AAV11, Rh10, Rh74, AAV-218, AAV-DJ, AAV-LK03, AAV-KP1, AAV-KP2, and AAV-KP3 capsid proteins, and variants thereof.

Still other aspects relate to a fusion protein encoded by the polynucleotide of any one of the preceding paragraphs.

Some aspects relate to a fusion protein comprising an amino acid sequence having at least 80%, at least 85%, at least 90%, at least 95%, or 100% identity to the amino acid sequence of any one of SEQ ID NOs: 72-84 and 88-90.

Other aspects relate to a host cell comprising the polynucleotide of any one of the preceding paragraphs, the vector of any one of the preceding paragraphs, the recombinant AAV vector genome of any one of the preceding paragraphs, the recombinant AAV vector of any one of the preceding paragraphs, or the fusion protein of any one of the preceding paragraphs.

Some aspects relate to method comprising administering to a pancreatic islet cell the polynucleotide of any one of the preceding paragraphs, the vector of any one of the preceding paragraphs, the recombinant AAV vector genome of any one of the preceding paragraphs, the recombinant AAV vector of any one of the preceding paragraphs, or the fusion protein of any one of the preceding paragraphs.

Other aspects relate to a method comprising administering to a subject the polynucleotide of any one of the preceding paragraphs, the vector of any one of the preceding paragraphs, the recombinant AAV vector genome of any one of the preceding paragraphs, the recombinant AAV vector of any one of the preceding paragraphs, or the fusion protein of any one of the preceding paragraphs, optionally wherein the subject has an obesity-related metabolic disorder selected from the group consisting of: prediabetes, Type 2 diabetes, cardiovascular disease, polycystic ovary syndrome (PCOS), non-alcoholic fatty liver disease (NAFLD), and non-alcoholic steatohepatitis (NASH).

Some aspects relate to a method of treating an obesity-related metabolic disorder, the method comprising administering to a subject in need thereof an effective amount of the polynucleotide of any one of the preceding paragraphs, the vector of any one of the preceding paragraphs, the recombinant AAV vector genome of any one of the preceding paragraphs, the recombinant AAV vector of any one of the preceding paragraphs, or the fusion protein of any one of the preceding paragraphs, optionally, wherein the obesity-related metabolic disorder is selected from the group consisting of: prediabetes, Type 2 diabetes, cardiovascular disease, polycystic ovary syndrome (PCOS), non-alcoholic fatty liver disease (NAFLD), and non-alcoholic steatohepatitis (NASH).

In some embodiments, the administering is via endoscopic delivery to the pancreas or a region near the pancreas, optionally wherein: (a) the effective amount restores glycemic durability in the subject; (b) the effective amount reduces fasting blood glucose by at least 50% relative to baseline; (c) the effective amount increases fasting insulin by at least 2-fold relative to baseline; (d) the effective amount significantly improves glucose tolerance relative to baseline; and/or (e) the effective amount significantly improves glucose-stimulated insulin secretion relative to baseline.

In some embodiments, body weight of the subject does not change significantly relative to baseline.

In some embodiments, the effective amount is a single dose, and the single dose is sufficient for long-term restoration of beta cell function and/or reduction in therapeutic burden.

Some aspects relate to an endoscopic gene therapy method, comprising: advancing a depositing device comprising at least one depositing element to at least one pancreatic deposit site in a patient having a metabolic disease and/or a pancreatic disease; and delivering an effective amount of a treatment agent through the at least one depositing element into the at least one pancreatic deposit site, wherein the treatment agent comprises the recombinant AAV vector genome of any one of the preceding paragraphs, or the recombinant AAV vector of any one of the preceding paragraphs.

In some embodiments, the at least one depositing element comprises at least one needle positioned on a distal portion of the depositing device.

In some embodiments, the distal end of the depositing device is delivered into the patient through the mouth, and advanced through a wall of the gastrointestinal tract to a location proximate the pancreas, optionally wherein (a) the depositing device is delivered through a working channel of a gastrointestinal endoscope that has been delivered through the mouth of the patient of (b) the depositing device is delivered alongside a gastrointestinal endoscope that has been delivered through the mouth of the patient.

In some embodiments: the metabolic disease is selected from the group consisting of: Type 1 diabetes; Type 2 diabetes; nonalcoholic fatty liver disease (NAFLD); nonalcoholic steatohepatitis (NASH); obesity; and combinations thereof; or the pancreatic disease is selected from the group consisting of: pancreatitis; pancreatic cancer; hyperinsulinism; and combinations thereof.

In some embodiments, the at least one pancreatic deposit site is selected from the group consisting of: intraparenchymal space; anterior pararenal space; intraductal space; intraarterial space of an artery that feeds at least a portion of the pancreas; and combinations thereof, preferably wherein the at least one pancreatic deposit site is the intraparenchymal space.

In some embodiments, the delivering of a treatment agent comprises at least a first delivery in which a minimum volume of the treatment agent is delivered into the pancreatic parenchyma, and wherein the minimum volume of treatment agent comprises a volume sufficient to cause at least a portion of the volume of the treatment agent to exit into the anterior pararenal space, spread, and re-enter the pancreas, optionally wherein the method further comprises at least a second delivery of the treatment agent to at least one additional deposit site proximate the tail of the pancreas.

In some embodiments, the delivering of a treatment agent comprises at least a first delivery in which a minimum volume of treatment agent is delivered into the pancreatic parenchyma, and wherein the minimum volume of the treatment agent comprises a volume of at least 2 ml, at least 3 ml, and/or at least 5 ml.

In some embodiments, the depositing device is advanced to the at least one pancreatic deposit site under image guidance, optionally, wherein the image guidance comprises: endoscopic ultrasound guidance; computerized tomography (CT) guidance; and/or magnetic Resonance Imaging (MRI) guidance.

In some embodiments, the at least one pancreatic deposit site comprises locations within 10 cm, 7.5 cm, 5 cm, and/or 3 cm of a portion of the pancreas, and wherein the portion of the pancreas comprises the tail, the neck, the body, the head, and/or the uncinate process.

In some embodiments, the treatment agent and/or the at least one depositing element is configured to be visualized by an imaging device, and wherein the method further comprises visualizing the treatment agent and/or the at least one depositing element with the imaging device to confirm proper delivery of the treatment agent.

In some embodiments, the method further comprises delivering an imaging agent through the at least one depositing element and visualizing the delivery of the imaging agent with an imaging device to confirm subsequently proper delivery of the treatment agent.

In some embodiments, the method further comprises pre-loading the depositing device with the treatment agent, optionally wherein the treatment agent is loaded into the depositing device from the distal end of the depositing device.

In some embodiments, the delivering of a treatment agent is performed at a pressure of at least 3 mmHg and/or no more than 25 mmHg.

In some embodiments, the delivering of a treatment agent is performed at a flow rate of at least 1 ml/min and/or no more than 5 ml/min.

In some embodiments, the at least one depositing element comprises multiple fenestrations along its length.

In some embodiments, the method further comprises confirming the at least one depositing element is in a proper location prior to delivering the treatment agent.

In some embodiments, the method further comprises the delivery of a permeability-enhancing agent prior to the delivery of the treatment agent and/or simultaneously with the delivery of the treatment agent, optionally wherein the delivery of the permeability-enhancing agent is performed locally and/or intravenously, optionally wherein the permeability-enhancing agent comprises an agent selected from the group consisting of: hyaluronidase; collagenase; losartan; and combinations thereof, and optionally wherein the treatment agent comprises a coformulation of the treatment agent and the permeability-enhancing agent.

In some embodiments, the method further comprises heating tissue proximate the at least one pancreatic deposit site to a temperature above 39° C. prior to, during, and/or after the delivery of the treatment agent.

In some embodiments, the method further comprises delivering a dissemination-blocking material that is configured to prevent undesired dissemination of the treatment agent to non-target locations, optionally wherein the dissemination-blocking material comprises a viscous substance and/or a polymer.

In some embodiments, the method further comprises positioning a blocking element in the patient, wherein the blocking element is configured to prevent undesired dissemination of the treatment agent to non-target locations.

In some embodiments, the method further comprises removing at least a portion of the treatment agent from a deposit site location after the delivery of the treatment agent begins.

In some embodiments, the method further comprises removing all of the treatment agent.

### BRIEF DESCRIPTION OF THE DRAWINGS

FIGS. 1A-1B show a schematic of endogenous insulin production (FIG. 1A) and corresponding subcellular localization (FIG. 1B).

FIGS. 2A-2B shows a schematic of AAV-based transgene production (FIG. 2A) and corresponding subcellular localization (FIG. 2B).

FIGS. 3A-3B show schematic representations of preproinsulin (INS) (FIG. 3A) and preproglucagon (GCG) (FIG. 3B).

FIG. 4 shows schematics of putative sites for insertion of a glucoregulatory hormone into the preprosinulin gene. Arrows indicate fusion sites.

FIG. 5 is a graph showing total GLP-1 detected in the supernatants of MIN-6 cells (a mouse insulinoma cell line) transfected with buffer only (TE Buffer), empty plasmids (pUC18 plasmid), plasmids expressing enhanced green fluorescence protein (GFP), plasmids expressing the human preproglucagon coding sequence (GCG), or plasmids expressing either INS-GLP-1 Hybrid #01, #02, #03, #04, #05, #06, #07, #08, #09, #10, #11, #12, or #13, 5-hours after a high glucose treatment (or high glucose (25 mM+0.100 mM 3-isobutyl-1-methylxanthine (IBMX)) treatment).

FIG. 6 is a graph showing fold-increase in total GLP-1 detected in the supernatants of MIN-6 cells transfected with cells only (control), empty plasmids (control), plasmids expressing GFP, plasmids expressing GCG, plasmids expressing INS-GLP-1 Hybrid #1, or plasmids expressing INS-GLP-1 Hybrid #2 after either low glucose (2 mM) or high glucose (25 mM+0.100 mM 3-isobutyl-1-methylxanthine (IBMX)) treatment.

FIG. 7 is a graph showing human insulin detected in the supernatants of MIN-6 cells transfected with cells only (control), empty plasmids (control), plasmids expressing GFP, plasmids expressing GCG, plasmids expressing INS-GLP-1 Hybrid #1, or plasmids expressing INS-GLP-1 Hybrid #2 after either low glucose (2 mM) or high glucose (25 mM+0.100 mM IBMX) treatment.

FIGS. 8A-8B are graphs showing total GLP-1 in the supernatants (FIG. 8A) and cell lysates (FIG. 8B) of EndoC-BH5 cells (human beta cells) 48-hours after transfection with various expression plasmids.

FIGS. 9A-9B are graphs showing dose-dependent sustained and reduced glycemia (FIG. 9A) and elevated fasting insulinemia (FIG. 9B). Statistics: ** P,0.01, *** P<0.001 vs. Vehicle, MIP-eGFP 10e12 groups; * P<0.05 vs vehicle only; One-Way ANOVA, post-hoc Tukey Test

FIGS. 10A-10C are graphs showing intraperitoneal glucose tolerance test (IPGTT) test results (FIG. 10A), the area under the curve (FIG. 10B), and insulin secretion (FIG. 10C) on Day 39. Statistics: in FIGS. 10A-10B, One-Way ANOVA; Post-hoc Tukey's multiple comparisons test, *P<0.05, **** P<0.0001. In FIG. 10C, Two-Way ANOVA mixed-effects model [REML]; Post-hoc Tukey's multiple comparison test, aP<0.005 vs. Vehicle, bP<0.05 vs. eGFP control.

FIGS. 11A-11B are graphs showing absolute body weight (FIG. 11A) and the change in body weight (FIG. 11B) over time.

FIGS. 12A-12C show that GLP-1RA protein (i.e., Exendin-4) is expressed in the pancreas via immunohistochemical staining (FIG. 12A), percent islet expression (FIG. 12B), and whole pancreas protein expression (FIG. 12C).

FIG. 13 shows PYY in the supernatants of MIN-6 cells transfected with various expression plasmids including control plasmids (empty and eGFP), the human PYY CDS, and INS-PYY Hybrids (#1-3) after either low glucose (2 mM) or high glucose (25 mM+0.01 mM IBMX) treatment (n=3 independent experiments).

FIGS. 14A-14B are graphs showing the identification of top functional GLP-1RA (i.e., Exendin-4) producers in the MIN-6 beta-cell line. FIG. 14A shows the total GLP-1RA secretion in a 25 mM glucose stimulation. FIG. 14B shows the cAMP signaling in a CHO-K1 hGLP-1R Gs cell line.

FIGS. 15A-15B are graphs showing improved insulin secretion from primary BKS db/db islets ex vivo after treatment with an AAV delivering a GLP-1RA (i.e., Exendin-4) (compared to an AAV-eGFP control). FIG. 15A shows the total GLP-1 content while FIG. 15B shows the glucose-stimulated insulin secretion.

FIG. 16 shows insulin levels following AAV-mediated delivery of GLP-1RA (i.e., Exendin-4) in the human beta-cell line EndoC-BH5. Exendin-9 (Ex9) peptide treatment, a potent inhibitor of the GLP-1R, demonstrates that increased INS secretion due to AAV-GLP-1RA is due to its action on the GLP-1R.

FIGS. 17A-17B are graphs showing the change in fasting blood glucose (FIG. 17A) and quantification of Exendin-4 in the serum and pancreas (FIG. 17B) of BKS db/db mice four weeks after administration of AAV-MIP-Ex-4 (an AAV-based Exendin-4 treatment) or vehicle.

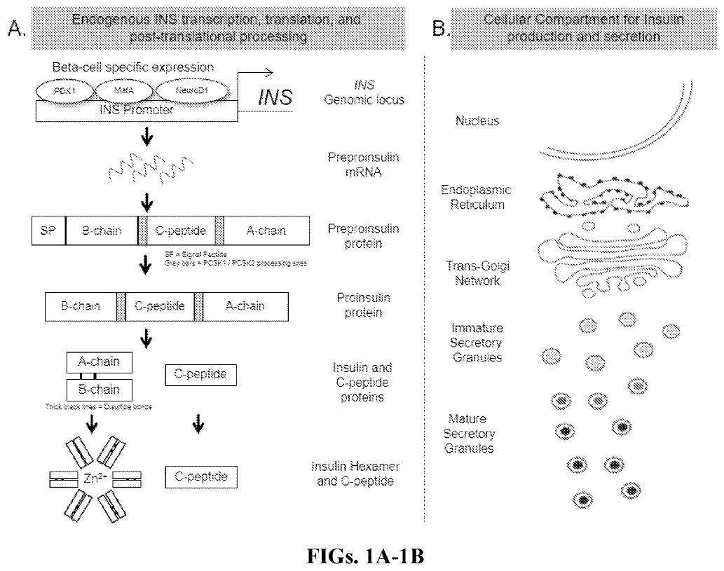

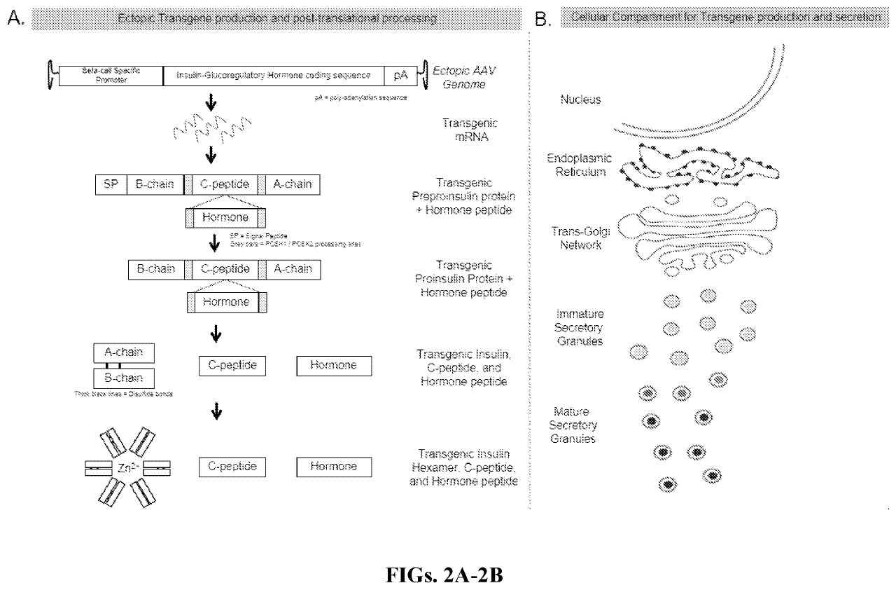

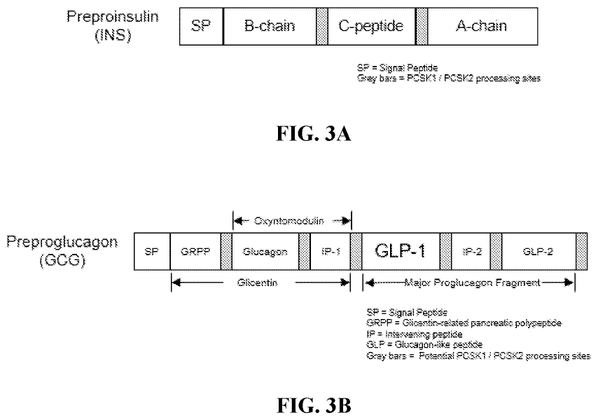

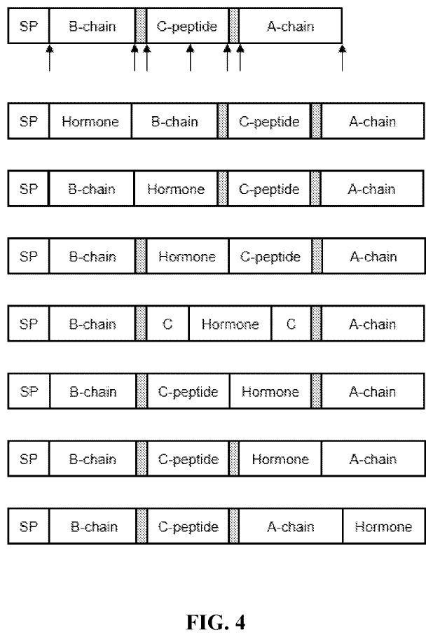

### DETAILED DESCRIPTION

Metabolic diseases result from a disruption of normal metabolism, the process of converting inputs (food and drink) into an output (energy). Typically, chemicals in the body break down the proteins, carbohydrates, and fats consumed, turning them into energy for current use or storing it for later use. Metabolic disorders, such as prediabetes, Type 2 diabetes (T2D), cardiovascular disease, polycystic ovary syndrome (PCOS), and non-alcoholic fatty liver disease (NAFLD), are conditions that increase the risk of heart disease, stroke, and death. The diseases are becoming more prevalent, and it is estimated that up to one-third of American adults have at least one. Despite advances in treatment over the last 50 years, metabolic diseases in general, and T2D in particular, continue to be a principal driver of morbidity and mortality today.

T2D is a disorder of rising blood glucose that is caused by a multitude of factors, which lead to two parallel, progressive disease processes within the body: insulin resistance and insulin insufficiency. Insulin resistance is the body's inability to respond appropriately to an insulin signal to remove glucose from the bloodstream, whereas insulin insufficiency is the gradual failure of the pancreas to produce sufficient insulin to meet the body's needs. Guidelines today focus on managing the blood glucose symptoms of T2D, often measured by blood concentrations of glycosylated hemoglobin, or HbA1c, rather than attempting to correct the underlying pathology in the body causing insulin resistance and insulin insufficiency. Therefore, patients make drastic dietary and lifestyle changes that require lifelong patient adherence and persistence to medicines. For some, this approach to care is unmanageable and leaves many patients at risk, potentially resulting in chronic elevations in blood glucose that increase the likelihood of microvascular and macrovascular complications of T2D, and even death.

There are no therapies that are approved today in T2D that offer disease modification, that is, ongoing and durable preservation of pancreatic insulin production capacity even after therapy is discontinued.

A new approach is described herein. Instead of treating a patient's symptoms, the compositions and methods described herein are used to treat the underlying causes of the disease with gene therapy. Described herein are gene therapy approaches to treating, with the goal of achieving long-term remission, metabolic disorders (e.g., T2D) by restoring insulin production. Briefly, gene therapy compositions and methods relating to key metabolic hormones necessary for proper insulin production in the beta cells of the pancreas are provided. As an example, a glucagon-like peptide-1 (GLP-1) coding sequence may be nested within an insulin (preproinsulin, proinsulin, or insulin) coding sequence, and the resulting polynucleotide may be delivered to the pancreas (e.g., in proximity to beta cells) by any means known the art. In one embodiment, the polynucleotide is packaged as an adeno-associated virus (AAV) vector and delivered locally using an endoscope. Without wishing to be bound by theory, it is thought that augmenting GLP-1 receptor activation in the pancreas may lead to reductions in blood glucose. Other configurations are also possible and described in greater detail below.

### Insulin-Glucoregulatory Hormone Polynucleotide

Provided herein, are polynucleotides comprising a glucoregulatory hormone coding sequence and an insulin (preproinsulin, proinsulin, or insulin) coding sequence. As used herein, “coding sequence” refers to a nucleotide sequence that directly defines the amino acid sequence of a protein product (i.e., the protein encoded by the nucleotide coding sequence). The limits of the coding sequence are generally determined by open reading frames (hereinafter “ORF”). An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein (e.g., a glucoregulatory hormone or insulin). The polynucleotide comprises nucleic acids (nucleotides). Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

As used herein, a “glucoregulatory hormone” refers to a hormone involved in the modulation of circulating blood glucose levels. Two hormones known canonically to be involved in blood glucose regulation are insulin, which lowers blood glucose levels, and glucagon, which elevates blood glucose levels. Thus, both insulin and glucagon are glucoregulatory hormones, and additional hormones that impact the secretion or function of insulin and/or glucagon may also be categorized as glucoregulatory hormones. In some embodiments, the glucoregulatory hormone acts directly; that is, it is directly involved in regulating blood glucose via insulin and/or glucagon modulation, and in other embodiments, the glucoregulatory hormone's effects are indirect; that is, the activity of the hormone indirectly affects blood glucose via insulin and/or glucagon modulation. Glucoregulatory hormones include, for example, preproglucagon-derived peptides (e.g., glucagon, GLP-1, oxyntomodulin, glicentin, glicentin-related polypeptide (GRPP), major proglucagon fragment, intervening peptide 1 (IP-1), intervening peptide 2 (IP-2), and GLP-2), incretins (e.g., GLP-1 and glucose-dependent insulinotropic peptide (GIP)), gut enteroendocrine cell derived peptides (e.g., GLP-1, peptide tyrosine (PYY), cholecystokinin (CCK), GIP, somatostatin, oxyntomodulin, and ghrelin), hormones produced in the pancreas (e.g., insulin, amylin, somatostatin, glucagon, and GLP-1). In some embodiments, the glucoregulatory hormone comprises an agent that mimics the action of GLP-1. In some embodiments, the agent comprises a GLP-1 receptor agonist (e.g., an agent that binds and activates GLP-1 receptors, reducing blood glucose levels). In some embodiments, the GLP-1 receptor agonist is a polypeptide agonist for the GLP-1 receptor (e.g., Exendin-4 and variants thereof). Exendin-4 (present in the saliva of the Gila monster, Heloderma suspectum) is a long acting GLP-1 analogue that is an agonist for the GLP-1 receptor. In some embodiments, the GLP-1 receptor agonist is Exendin-4. Other endocrine hormones with glucoregulatory activity contemplated herein include, without limitation, leptin, follistatin, insulin-like growth factor 1 (IGF1), vasoactive intestinal peptide (VIP), and growth hormone 1 (GH1). See Table 1 for exemplary protein and coding sequences.

TABLE 1
Examples of Glucoregulatory Hormone Coding Sequences and Proteins Sequences

| Name | Nucleic acid | SEQ ID NO: |
|---|---|---|
| GCG | ATGAAAAGCATTTACTTTGTGGCTGGGTTATTTGTAATGCTGGTAC | 1 |
| (Preproglucagon) | AAGGCAGCTGGCAACGTTCCCTTCAAGACACAGAGGAGAAATCCAG | |
| | ATCATTCTCAGCTTCCCAGGCAGACCCACTCAGTGATCCTGATCAG | |
| | ATGAACGAGGACAAGCGCCATTCACAGGGCACATTCACCAGTGACT | |
| | ACAGCAAGTATCTGGACTCCAGGCGTGCCCAAGATTTTGTGCAGTG | |
| | GTTGATGAATACCAAGAGGAACAGGAATAACATTGCCAAACGTCAC | |
| | GATGAATTTGAGAGACATGCTGAAGGGACCTTTACCAGTGATGTAA | |
| | GTTCTTATTTGGAAGGCCAAGCTGCCAAGGAATTCATTGCTTGGCT | |
| | GGTGAAAGGCCGAGGAAGGCGAGATTTCCCAGAAGAGGTCGCCATT | |
| | GTTGAAGAACTTGGCCGCAGACATGCTGATGGTTCTTTCTCTGATG | |
| | AGATGAACACCATTCTTGATAATCTTGCCGCCAGGGACTTTATAAA | |
| | CTGGTTGATTCAGACCAAAATCACTGACAGGAAATAA | |
| Glucagon | CATTCACAGGGCACATTCACCAGTGACTACAGCAAGTATCTGGACT | 2 |
| | CCAGGCGTGCCCAAGATTTTGTGCAGTGGTTGATGAATACC | |
| GLP-1(1-37) | CACGATGAATTTGAGAGACATGCTGAAGGGACCTTTACCAGTGATG | 3 |
| | TAAGTTCTTATTTGGAAGGCCAAGCTGCCAAGGAATTCATTGCTTG | |
| | GCTGGTGAAAGGCCGAGGA | |
| Oxyntomodulin | CATTCACAGGGCACATTCACCAGTGACTACAGCAAGTATCTGGACT | 4 |
| | CCAGGCGTGCCCAAGATTTTGTGCAGTGGTTGATGAATACCAAGAG | |
| | GAACAGGAATAACATTGCC | |
| Glicentin | CGTTCCCTTCAAGACACAGAGGAGAAATCCAGATCATTCTCAGCTT | 5 |
| | CCCAGGCAGACCCACTCAGTGATCCTGATCAGATGAACGAGGACAA | |
| | GCGCCATTCACAGGGCACATTCACCAGTGACTACAGCAAGTATCTG | |
| | GACTCCAGGCGTGCCCAAGATTTTGTGCAGTGGTTGATGAATACCA | |
| | AGAGGAACAGGAATAACATTGCC | |
| Glicentin-related | CGTTCCCTTCAAGACACAGAGGAGAAATCCAGATCATTCTCAGCTT | 6 |
| polypeptide (GRPP) | CCCAGGCAGACCCACTCAGTGATCCTGATCAGATGAACGAGGAC | |
| Major proglucagon | CACGATGAATTTGAGAGACATGCTGAAGGGACCTTTACCAGTGATG | 7 |
| fragment | TAAGTTCTTATTTGGAAGGCCAAGCTGCCAAGGAATTCATTGCTTG | |
| | GCTGGTGAAAGGCCGAGGAAGGCGAGATTTCCCAGAAGAGGTCGCC | |
| | ATTGTTGAAGAACTTGGCCGCAGACATGCTGATGGTTCTTTCTCTG | |
| | ATGAGATGAACACCATTCTTGATAATCTTGCCGCCAGGGACTTTAT | |
| | AAACTGGTTGATTCAGACCAAAATCACTGAC | |
| Intervening peptide 1 | AACAGGAATAACATTGCC | 8 |
| (IP-1) | | |
| Intervening peptide 2 | GATTTCCCAGAAGAGGTCGCCATTGTTGAAGAACTTGGC | 9 |
| (IP-2) | | |
| GLP-2 | CATGCTGATGGTTCTTTCTCTGATGAGATGAACACCATTCTTGATA | 10 |
| | ATCTTGCCGCCAGGGACTTTATAAACTGGTTGATTCAGACCAAAAT | |
| | CACTGAC | |
| Glucose-dependent | ATGGTGGCCACGAAGACCTTTGCTCTGCTGCTGCTGTCCCTGTTCC | 11 |
| insulinotropic peptide | TGGCAGTGGGACTAGGAGAGAAGAAAGAGGGTCACTTCAGCGCTCT | |
| (GIP) | CCCCTCCCTGCCTGTTGGATCTCATGCTAAGGTGAGCAGCCCTCAA | |
| | CCTCGAGGCCCCAGGTACGCGGAAGGGACTTTCATCAGTGACTACA | |
| | GTATTGCCATGGACAAGATTCACCAACAAGACTTTGTGAACTGGCT | |
| | GCTGGCCCAAAAGGGGAAGAAGAATGACTGGAAACACAACATCACC | |
| | CAGAGGGAGGCTCGGGCGCTGGAGCTGGCCAGTCAAGCTAATAGGA | |
| | AGGAGGAGGAGGCAGTGGAGCCACAGAGCTCCCCAGCCAAGAACCC | |
| | CAGCGATGAAGATTTGCTGCGGGACTTGCTGATTCAAGAGCTGTTG | |
| | GCCTGCTTGCTGGATCAGACAAACCTCTGCAGGCTCAGGTCTCGGT | |
| | GA | |
| Peptide tyrosine- | ATGGTGTTCGTGCGCAGGCCGTGGCCCGCCTTGACCACAGTGCTTC | 12 |
| tyrosine (PYY) | TGGCCCTGCTCGTCTGCCTAGGGGCGCTGGTCGACGCCTACCCCAT | |
| | CAAACCCGAGGCTCCCCGCGAAGACGCCTCGCCGGAGGAGCTGAAC | |
| | CGCTACTACGCCTCCCTGCGCCACTACCTCAACCTGGTCACCCGGC | |
| | AGCGGTATGGGAAAAGAGACGGCCCGGACACGCTTCTTTCCAAAAC | |
| | GTTCTTCCCCGACGGCGAGGACCGCCCCGTCAGGTCGCGGTCGGAG | |
| | GGCCCAGACCTGTGGTGA | |
| Peptide tyrosine- | TACCCCATCAAACCCGAGGCTCCCCGCGAAGACGCCTCGCCGGAGG | 91 |
| tyrosine (PYY) | AGCTGAACCGCTACTACGCCTCCCTGCGCCACTACCTCAACCTGGT | |
| (Y29-R67) | CACCCGGCAGCGGTATGGGAAAAGA | |
| Peptide tyrosine- | TACCCCATCAAACCCGAGGCTCCCCGCGAAGACGCCTCGCCGGAGG | 92 |
| tyrosine (PYY) | AGCTGAACCGCTACTACGCCTCCCTGCGCCACTACCTCAACCTGGT | |
| (Y29-R67) | CACCCGGCAGCGGTAT | |
| Cholecystokinin | ATGAACAGCGGCGTGTGCCTGTGCGTGCTGATGGCGGTACTGGCGG | 13 |
| (CCK) | CTGGCGCCCTGACGCAGCCGGTGCCTCCCGCAGATCCCGCGGGCTC | |
| | CGGGCTGCAGCGGGCAGAGGAGGCGCCCCGTAGGCAGCTGAGGGTA | |
| | TCGCAGAGAACGGATGGCGAGTCCCGAGCGCACCTGGGCGCCCTGC | |
| | TGGCAAGATACATCCAGCAGGCCCGGAAAGCTCCTTCTGGACGAAT | |
| | GTCCATCGTTAAGAACCTGCAGAACCTGGACCCCAGCCACAGGATA | |
| | AGTGACCGGGACTACATGGGCTGGATGGATTTTGGCCGTCGCAGTG | |
| | CCGAGGAGTATGAGTACCCCTCCTAG | |
| Somatostatin | ATGCTGTCCTGCCGCCTCCAGTGCGCGCTGGCTGCGCTGTCCATCG | 14 |
| | TCCTGGCCCTGGGCTGTGTCACCGGCGCTCCCTCGGACCCCAGACT | |
| | CCGTCAGTTTCTGCAGAAGTCCCTGGCTGCTGCCGCGGGGAAGCAG | |
| | GAACTGGCCAAGTACTTCTTGGCAGAGCTGCTGTCTGAACCCAACC | |
| | AGACGGAGAATGATGCCCTGGAACCTGAAGATCTGTCCCAGGCTGC | |
| | TGAGCAGGATGAAATGAGGCTTGAGCTGCAGAGATCTGCTAACTCA | |
| | AACCCGGCTATGGCACCCCGAGAACGCAAAGCTGGCTGCAAGAATT | |
| | TCTTCTGGAAGACTTTCACATCCTGTTAG | |
| Ghrelin | ATGCCCTCCCCAGGGACCGTCTGCAGCCTCCTGCTCCTCGGCATGC | 15 |
| | TCTGGCTGGACTTGGCCATGGCAGGCTCCAGCTTCCTGAGCCCTGA | |
| | ACACCAGAGAGTCCAGAGAAAGGAGTCGAAGAAGCCACCAGCCAAG | |
| | CTGCAGCCCCGAGCTCTAGCAGGCTGGCTCCGCCCGGAAGATGGAG | |
| | GTCAAGCAGAAGGGGCAGAGGATGAACTGGAAGTCCGGTTCAACGC | |
| | CCCCTTTGATGTTGGAATCAAGCTGTCAGGGGTTCAGTACCAGCAG | |
| | CACAGCCAGGCCCTGGGGAAGTTTCTTCAGGACATCCTCTGGGAAG | |
| | AGGCCAAAGAGGCCCCAGCCGACAAGTGA | |
| Insulin | ATGGCCCTGTGGATGCGCCTCCTGCCCCTGCTGGCGCTGCTGGCCC | 16 |
| | TCTGGGGACCTGACCCAGCCGCAGCCTTTGTGAACCAACACCTGTG | |
| | CGGCTCACACCTGGTGGAAGCTCTCTACCTAGTGTGCGGGGAACGA | |
| | GGCTTCTTCTACACACCCAAGACCCGCCGGGAGGCAGAGGACCTGC | |
| | AGGTGGGGCAGGTGGAGCTGGGCGGGGGCCCTGGTGCAGGCAGCCT | |
| | GCAGCCCTTGGCCCTGGAGGGGTCCCTGCAGAAGCGTGGCATTGTG | |
| | GAACAATGCTGTACCAGCATCTGCTCCCTCTACCAGCTGGAGAACT | |
| | ACTGCAACTAG | |
| Amylin | ATGGGCATCCTGAAGCTGCAAGTATTTCTCATTGTGCTCTCTGTTG | 17 |
| | CATTGAACCATCTGAAAGCTACACCCATTGAAAGTCATCAGGTGGA | |
| | AAAGCGGAAATGCAACACTGCCACATGTGCAACGCAGCGCCTGGCA | |
| | AATTTTTTAGTTCATTCCAGCAACAACTTTGGTGCCATTCTCTCAT | |
| | CTACCAACGTGGGATCCAATACATATGGCAAGAGGAATGCAGTAGA | |
| | GGTTTTAAAGAGAGAGCCACTGAATTACTTGCCCCTTTAG | |
| Leptin | ATGCATTGGGGAACCCTGTGCGGATTCTTGTGGCTTTGGCCCTATC | 18 |
| | TTTTCTATGTCCAAGCTGTGCCCATCCAAAAAGTCCAAGATGACAC | |
| | CAAAACCCTCATCAAGACAATTGTCACCAGGATCAATGACATTTCA | |
| | CACACGCAGTCAGTCTCCTCCAAACAGAAAGTCACCGGTTTGGACT | |
| | TCATTCCTGGGCTCCACCCCATCCTGACCTTATCCAAGATGGACCA | |
| | GACACTGGCAGTCTACCAACAGATCCTCACCAGTATGCCTTCCAGA | |
| | AACGTGATCCAAATATCCAACGACCTGGAGAACCTCCGGGATCTTC | |
| | TTCACGTGCTGGCCTTCTCTAAGAGCTGCCACTTGCCCTGGGCCAG | |
| | TGGCCTGGAGACCTTGGACAGCCTGGGGGGTGTCCTGGAAGCTTCA | |
| | GGCTACTCCACAGAGGTGGTGGCCCTGAGCAGGCTGCAGGGGTCTC | |
| | TGCAGGACATGCTGTGGCAGCTGGACCTCAGCCCTGGGTGCTGA | |
| Follistatin | ATGGTCCGCGCGAGGCACCAGCCGGGTGGGCTTTGCCTCCTGCTGC | 19 |
| | TGCTGCTCTGCCAGTTCATGGAGGACCGCAGTGCCCAGGCTGGGAA | |
| | CTGCTGGCTCCGTCAAGCGAAGAACGGCCGCTGCCAGGTCCTGTAC | |
| | AAGACCGAACTGAGCAAGGAGGAGTGCTGCAGCACCGGCCGGCTGA | |
| | GCACCTCGTGGACCGAGGAGGACGTGAATGACAACACACTCTTCAA | |
| | GTGGATGATTTTCAACGGGGGCGCCCCCAACTGCATCCCCTGTAAA | |
| | GAAACGTGTGAGAACGTGGACTGTGGACCTGGGAAAAAATGCCGAA | |
| | TGAACAAGAAGAACAAACCCCGCTGCGTCTGCGCCCCGGATTGTTC | |
| | CAACATCACCTGGAAGGGTCCAGTCTGCGGGCTGGATGGGAAAACC | |
| | TACCGCAATGAATGTGCACTCCTAAAGGCAAGATGTAAAGAGCAGC | |
| | CAGAACTGGAAGTCCAGTACCAAGGCAGATGTAAAAAGACTTGTCG | |
| | GGATGTTTTCTGTCCAGGCAGCTCCACATGTGTGGTGGACCAGACC | |
| | AATAATGCCTACTGTGTGACCTGTAATCGGATTTGCCCAGAGCCTG | |
| | CTTCCTCTGAGCAATATCTCTGTGGGAATGATGGAGTCACCTACTC | |
| | CAGTGCCTGCCACCTGAGAAAGGCTACCTGCCTGCTGGGCAGATCT | |
| | ATTGGATTAGCCTATGAGGGAAAGTGTATCAAAGCAAAGTCCTGTG | |
| | AAGATATCCAGTGCACTGGTGGGAAAAAATGTTTATGGGATTTCAA | |
| | GGTTGGGAGAGGCCGGTGTTCCCTCTGTGATGAGCTGTGCCCTGAC | |
| | AGTAAGTCGGATGAGCCTGTCTGTGCCAGTGACAATGCCACTTATG | |
| | CCAGCGAGTGTGCCATGAAGGAAGCTGCCTGCTCCTCAGGTGTGCT | |
| | ACTGGAAGTAAAGCACTCCGGATCTTGCAACTGA | |
| Insulin-like growth | ATGGGAAAAATCAGCAGTCTTCCAACCCAATTATTTAAGTGCTGCT | 20 |
| factor 1 (IGF1) | TTTGTGATTTCTTGAAGGTGAAGATGCACACCATGTCCTCCTCGCA | |
| | TCTCTTCTACCTGGCGCTGTGCCTGCTCACCTTCACCAGCTCTGCC | |
| | ACGGCTGGACCGGAGACGCTCTGCGGGGCTGAGCTGGTGGATGCTC | |
| | TTCAGTTCGTGTGTGGAGACAGGGGCTTTTATTTCAACAAGCCCAC | |
| | AGGGTATGGCTCCAGCAGTCGGAGGGCGCCTCAGACAGGCATCGTG | |
| | GATGAGTGCTGCTTCCGGAGCTGTGATCTAAGGAGGCTGGAGATGT | |
| | ATTGCGCACCCCTCAAGCCTGCCAAGTCAGCTCGCTCTGTCCGTGC | |
| | CCAGCGCCACACCGACATGCCCAAGACCCAGAAGGAAGTACATTTG | |
| | AAGAACGCAAGTAGAGGGAGTGCAGGAAACAAGAACTACAGGATGT | |
| | AG | |
| Vasoactive intestinal | ATGGACACCAGAAATAAGGCCCAGCTCCTTGTGCTCCTGACTCTTC | 21 |
| peptide (VIP) | TCAGTGTGCTCTTCTCACAGACTTCGGCATGGCCTCTTTACAGGGC | |
| | ACCTTCTGCTCTCAGGTTGGGTGACAGAATACCCTTTGAGGGAGCA | |
| | AATGAACCTGATCAAGTTTCATTAAAAGAAGACATTGACATGTTGC | |
| | AAAATGCATTAGCTGAAAATGACACACCCTATTATGATGTATCCAG | |
| | AAATGCCAGGCATGCTGATGGAGTTTTCACCAGTGACTTCAGTAAA | |
| | CTCTTGGGTCAACTTTCTGCCAAAAAGTACCTTGAGTCTCTTATGG | |
| | GAAAACGTGTTAGCAGTAACATCTCAGAAGACCCTGTACCAGTCAA | |
| | ACGTCACTCAGATGCAGTCTTCACTGACAACTATACCCGCCTTAGA | |
| | AAACAAATGGCTGTAAAGAAATATTTGAACTCAATTCTGAATGGAA | |
| | AGAGGAGCAGTGAGGGAGAATCTCCCGACTTTCCAGAAGAGTTAGA | |
| | AAAATGA | |
| Growth hormone 1 | ATGGCTACAGGCTCCCGGACGTCCCTGCTCCTGGCTTTTGGCCTGC | 22 |
| (GH1) | TCTGCCTGCCCTGGCTTCAAGAGGGCAGTGCCTTCCCAACCATTCC | |
| | CTTATCCAGGCTTTTTGACAACGCTATGCTCCGCGCCCATCGTCTG | |
| | CACCAGCTGGCCTTTGACACCTACCAGGAGTTTGAAGAAGCCTATA | |
| | TCCCAAAGGAACAGAAGTATTCATTCCTGCAGAACCCCCAGACCTC | |
| | CCTCTGTTTCTCAGAGTCTATTCCGACACCCTCCAACAGGGAGGAA | |
| | ACACAACAGAAATCCAACCTAGAGCTGCTCCGCATCTCCCTGCTGC | |
| | TCATCCAGTCGTGGCTGGAGCCCGTGCAGTTCCTCAGGAGTGTCTT | |
| | CGCCAACAGCCTGGTGTACGGCGCCTCTGACAGCAACGTCTATGAC | |
| | CTCCTAAAGGACCTAGAGGAAGGCATCCAAACGCTGATGGGGAGGC | |
| | TGGAAGATGGCAGCCCCCGGACTGGGCAGATCTTCAAGCAGACCTA | |
| | CAGCAAGTTCGACACAAACTCACACAACGATGACGCACTACTCAAG | |
| | AACTACGGGCTGCTCTACTGCTTCAGGAAGGACATGGACAAGGTCG | |
| | AGACATTCCTGCGCATCGTGCAGTGCCGCTCTGTGGAGGGCAGCTG | |
| | TGGCTTCTAG | |
| GLP-1(7-37) | CATGCTGAAGGGACCTTTACCAGTGATGTAAGTTCTTATTTGGAAG | 23 |
| | GCCAAGCTGCCAAGGAATTCATTGCTTGGCTGGTGAAAGGCCGAGG | |
| | A | |
| GCG(K82-R145)/ | AAGAGGAACAGGAATAACATTGCCAAACGTCACGATGAATTTGAGA | 24 |
| GLP-1(K82-R145) | GACATGCTGAAGGGACCTTTACCAGTGATGTAAGTTCTTATTTGGA | |
| | AGGCCAAGCTGCCAAGGAATTCATTGCTTGGCTGGTGAAAGGCCGA | |
| | GGAAGGCGAGATTTCCCAGAAGAGGTCGCCATTGTTGAAGAACTTG | |
| | GCCGCAGA | |
| GCG(K90-R130)/ | AAACGTCACGATGAATTTGAGAGACATGCTGAAGGGACCTTTACCA | 25 |
| GLP-1(K90-R130) | GTGATGTAAGTTCTTATTTGGAAGGCCAAGCTGCCAAGGAATTCAT | |
| | TGCTTGGCTGGTGAAAGGCCGAGGAAGGCGA | |
| GCG(K82-R130)/ | AAGAGGAACAGGAATAACATTGCCAAACGTCACGATGAATTTGAGA | 26 |
| GLP-1(K82-R130) | GACATGCTGAAGGGACCTTTACCAGTGATGTAAGTTCTTATTTGGA | |
| | AGGCCAAGCTGCCAAGGAATTCATTGCTTGGCTGGTGAAAGGCCGA | |
| | GGAAGGCGA | |
| GCG(K82-R145)/ | AAGAGGAACAGGAATAACATTGCCAAACGTCACGATGAATTTGAGA | 27 |
| GLP-1(K82-R145) | GACATGCTGAAGGGACCTTTACCAGTGATGTAAGTTCTTATTTGGA | |
| | AGGCCAAGCTGCCAAGGAATTCATTGCTTGGCTGGTGAAAGGCCGA | |
| | GGAAGGCGAGATTTCCCAGAAGAGGTCGCCATTGTTGAAGAACTTG | |
| | GCCGCAGA | |
| GCG(K90-R130)/ | AAACGTCACGATGAATTTGAGAGACATGCTGAAGGGACCTTTACCA | 28 |
| GLP-1(K90-R130) | GTGATGTAAGTTCTTATTTGGAAGGCCAAGCTGCCAAGGAATTCAT | |
| | TGCTTGGCTGGTGAAAGGCCGAGGAAGGCGA | |
| GCG(K82-P133)/ | AAACGTCACGATGAATTTGAGAGACATGCTGAAGGGACCTTTACCA | 29 |
| GLP-1(K82-P133) | GTGATGTAAGTTCTTATTTGGAAGGCCAAGCTGCCAAGGAATTCAT | |
| | TGCTTGGCTGGTGAAAGGCCGAGGAAGGCGAGATTTCCCAGAAGAG | |
| | GTCGCCATTGTTGAAGAACTTGGCCGCAGA | |
| GCG(K90-R145)/ | AAGAGGAACAGGAATAACATTGCCAAACGTCACGATGAATTTGAGA | 30 |
| GLP-1(K90-R145) | GACATGCTGAAGGGACCTTTACCAGTGATGTAAGTTCTTATTTGGA | |
| | AGGCCAAGCTGCCAAGGAATTCATTGCTTGGCTGGTGAAAGGCCGA | |
| | GGAAGGCGAGATTTCCCA | |
| Name | Amino Acid | SEQ ID NO: |
| GCG (Preproglucagon) | MKSIYFVAGLFVMLVQGSWQRSLQDTEEKSRSFSASQADPLSDPDQ | 31 |
| | MNEDKRHSQGTFTSDYSKYLDSRRAQDFVQWLMNTKRNRNNIAKRH | |
| | DEFERHAEGTFTSDVSSYLEGQAAKEFIAWLVKGRGRRDFPEEVAI | |
| | VEELGRRHADGSFSDEMNTILDNLAARDFINWLIQTKITDRK | |
| Glucagon | HSQGTFTSDYSKYLDSRRAQDFVQWLMNT | 32 |
| GLP-1(1-37) | HDEFERHAEGTFTSDVSSYLEGQAAKEFIAWLVKGRG | 33 |
| Oxyntomodulin | HSQGTFTSDYSKYLDSRRAQDFVQWLMNTKRNRNNIA | 34 |
| Glicentin | RSLQDTEEKSRSFSASQADPLSDPDQMNEDKRHSQGTFTSDYSKYL | 35 |
| | DSRRAQDFVQWLMNTKRNRNNIA | |
| Glicentin-related | RSLQDTEEKSRSFSASQADPLSDPDQMNED | 36 |
| polypeptide (GRPP) | | |
| Major proglucagon | HDEFERHAEGTFTSDVSSYLEGQAAKEFIAWLVKGRGRRDFPEEVA | 37 |
| fragment | IVEELGRRHADGSFSDEMNTILDNLAARDFINWLIQTKITD | |
| Intervening peptide 1 | NRNNIA | 38 |
| (IP-1) | | |
| Intervening peptide 2 | DFPEEVAIVEELG | 39 |
| (IP-2) | | |
| GLP-2 | HADGSFSDEMNTILDNLAARDFINWLIQTKITD | 40 |
| Glucose-dependent | MVATKTFALLLLSLFLAVGLGEKKEGHFSALPSLPVGSHAKVSSPQ | 41 |
| insulinotropic peptide | PRGPRYAEGTFISDYSIAMDKIHQQDFVNWLLAQKGKKNDWKHNIT | |
| (GIP) | QREARALELASQANRKEEEAVEPQSSPAKNPSDEDLLRDLLIQELL | |
| | ACLLDQTNLCRLRSR | |
| Peptide tyrosine- | MVFVRRPWPALTTVLLALLVCLGALVDAYPIKPEAPREDASPEELN | 42 |
| tyrosine (PYY) | RYYASLRHYLNLVTRQRYGKRDGPDTLLSKTFFPDGEDRPVRSRSE | |
| | GPDLW | |
| Peptide tyrosine- | YPIKPEAPREDASPEELNRYYASLRHYLNLVTRQRYGKR | 93 |
| tyrosine (PYY) | | |
| (Y29-R67) | | |
| Peptide tyrosine- | YPIKPEAPREDASPEELNRYYASLRHYLNLVTRQRY | 94 |
| tyrosine (PYY) (1-36) | | |
| Cholecystokinin | MNSGVCLCVLMAVLAAGALTQPVPPADPAGSGLQRAEEAPRRQLRV | 43 |
| (CCK) | SQRTDGESRAHLGALLARYIQQARKAPSGRMSIVKNLQNLDPSHRI | |
| | SDRDYMGWMDFGRRSAEEYEYPS | |
| Somatostatin | MLSCRLQCALAALSIVLALGCVTGAPSDPRLRQFLQKSLAAAAGKQ | 44 |
| | ELAKYFLAELLSEPNQTENDALEPEDLSQAAEQDEMRLELQRSANS | |
| | NPAMAPRERKAGCKNFFWKTFTSC | |
| Ghrelin | MPSPGTVCSLLLLGMLWLDLAMAGSSFLSPEHQRVQRKESKKPPAK | 45 |
| | LQPRALAGWLRPEDGGQAEGAEDELEVRENAPFDVGIKLSGVQYQQ | |
| | HSQALGKFLQDILWEEAKEAPADK | |
| Insulin | MALWMRLLPLLALLALWGPDPAAAFVNQHLCGSHLVEALYLVCGER | 46 |
| | GFFYTPKTRREAEDLQVGQVELGGGPGAGSLQPLALEGSLQKRGIV | |
| | EQCCTSICSLYQLENYCN* | |
| Amylin | MGILKLQVFLIVLSVALNHLKATPIESHQVEKRKCNTATCATQRLA | 47 |
| | NFLVHSSNNFGAILSSTNVGSNTYGKRNAVEVLKREPLNYLPL | |
| Leptin | MHWGTLCGFLWLWPYLFYVQAVPIQKVQDDTKTLIKTIVTRINDIS | 48 |
| | HTQSVSSKQKVTGLDFIPGLHPILTLSKMDQTLAVYQQILTSMPSR | |
| | NVIQISNDLENLRDLLHVLAFSKSCHLPWASGLETLDSLGGVLEAS | |
| | GYSTEVVALSRLQGSLQDMLWQLDLSPGC | |
| Follistatin | MVRARHQPGGLCLLLLLLCQFMEDRSAQAGNCWLRQAKNGRCQVLY | 49 |
| | KTELSKEECCSTGRLSTSWTEEDVNDNTLFKWMIFNGGAPNCIPCK | |
| | ETCENVDCGPGKKCRMNKKNKPRCVCAPDCSNITWKGPVCGLDGKT | |
| | YRNECALLKARCKEQPELEVQYQGRCKKTCRDVFCPGSSTCVVDQT | |
| | NNAYCVTCNRICPEPASSEQYLCGNDGVTYSSACHLRKATCLLGRS | |
| | IGLAYEGKCIKAKSCEDIQCTGGKKCLWDFKVGRGRCSLCDELCPD | |
| | SKSDEPVCASDNATYASECAMKEAACSSGVLLEVKHSGSCN | |
| Insulin-like growth | MGKISSLPTQLFKCCFCDFLKVKMHTMSSSHLFYLALCLLTFTSSA | 50 |
| factor 1 (IGF1) | TAGPETLCGAELVDALQFVCGDRGFYENKPTGYGSSSRRAPQTGIV | |
| | DECCFRSCDLRRLEMYCAPLKPAKSARSVRAQRHTDMPKTQKEVHL | |
| | KNASRGSAGNKNYRM | |
| Vasoactive intestinal | MDTRNKAQLLVLLTLLSVLFSQTSAWPLYRAPSALRLGDRIPFEGA | 51 |
| peptide (VIP) | NEPDQVSLKEDIDMLQNALAENDTPYYDVSRNARHADGVFTSDFSK | |
| | LLGQLSAKKYLESLMGKRVSSNISEDPVPVKRHSDAVFTDNYTRLR | |
| | KQMAVKKYLNSILNGKRSSEGESPDFPEELEK | |
| Growth hormone 1 | MATGSRTSLLLAFGLLCLPWLQEGSAFPTIPLSRLEDNAMLRAHRL | 52 |
| (GH1) | HQLAFDTYQEFEEAYIPKEQKYSFLQNPQTSLCFSESIPTPSNREE | |
| | TQQKSNLELLRISLLLIQSWLEPVQFLRSVFANSLVYGASDSNVYD | |
| | LLKDLEEGIQTLMGRLEDGSPRTGQIFKQTYSKFDTNSHNDDALLK | |
| | NYGLLYCFRKDMDKVETFLRIVQCRSVEGSCGF | |
| GLP-1(7-37) | HAEGTFTSDVSSYLEGQAAKEFIAWLVKGRG | 53 |
| GCG(K82-R130)/ | KRNRNNIAKRHDEFERHAEGTFTSDVSSYLEGQAAKEFIAWLVKGR | 54 |
| GLP-1(K82-R130) | GRR | |
| GCG(K82-R145)/ | KRNRNNIAKRHDEFERHAEGTFTSDVSSYLEGQAAKEFIAWLVKGR | 55 |
| GLP-1(K82-R145) | GRRDFPEEVAIVEELGRR | |
| GCG(K90-R130)/ | KRHDEFERHAEGTFTSDVSSYLEGQAAKEFIAWLVKGRGRR | 56 |
| GLP-1(K90-R130) | | |
| GCG(K82-P133)/ | KRHDEFERHAEGTFTSDVSSYLEGQAAKEFIAWLVKGRGRRDFPEE | 57 |
| GLP-1(K82-P133) | VAIVEELGRR | |
| GCG(K90-R145)/ | KRNRNNIAKRHDEFERHAEGTFTSDVSSYLEGQAAKEFIAWLVKGR | 58 |
| GLP-1(K90-R145) | GRRDFP | |

It should be understood that the term “glucoregulatory hormone” includes functional peptides and polypeptides of any of the foregoing examples as well as functional variants thereof, meaning the peptides and/or variants are capable of impacting blood glucose control through either direct or indirect functions.

Thus, amino acid modifications (e.g., substitutions) can be made to the glucoregulatory hormones provided herein. In some embodiments, modified amino acid sequence imparts a beneficial property for protein production and/or function. For example, a GLP-1 peptide sequence can include a glycine substitution for alanine at the amino acid position 8 of the GLP-1(1-37) sequence (GLP-1-Gly8), which confers resistance to cleavage into an inactive form by dipeptidyl peptidase-IV (DPP4 or DPPIV). Other modifications and thus other variants are contemplated herein.

In some embodiments, the polynucleotide comprises multiple coding sequences, each for a different protein. In some embodiments, the polynucleotide comprises one or more coding sequence(s) for 1-10 glucoregulatory hormones. For example, a polynucleotide may comprise one or more coding sequence(s) for 1-3, 1-4, or 1-5 different glucoregulatory hormones. In some embodiments, the polynucleotide comprises one or more coding sequence(s) 1, 2, 3, 4, 5, 6, 7, 8, 9, 10, or more glucoregulatory hormones. In some embodiments, the polynucleotide comprises a GLP-1 coding sequence. In some embodiments, the polynucleotide comprises a GLP-1 coding sequence, an IP-1 coding sequence and/or an IP-2 coding sequence.

GLP-1 is a polypeptide derived from the proglucagon protein. Under physiological conditions, it is produced and secreted by intestinal enteroendocrine L-cells and certain neurons within the nucleus of the solitary tract in the brainstem upon food consumption. GLP-1 is rapidly metabolized and inactivated by dipeptidyl peptidase IV (an enzyme) even before the hormone has left the gut. GLP-1 stimulates insulin secretion (acting as an incretin hormone) and inhibits glucagon secretion. It also inhibits gastrointestinal motility and secretion. In this way, the protein acts as an enterogastrone and part of the “ileal brake” mechanism. GLP-1 also plays a role as a physiological regulator of appetite and food intake. Decreased secretion of GLP-1 can lead to the development of obesity. In some embodiments, the polynucleotide encodes a full-length GLP-1 sequence (e.g., SEQ ID NO: 33) or a truncated GLP-1 sequence (e.g., any one of SEQ ID NOS: 53-58), or other functional variant or fragment thereof.

The polynucleotides described herein also comprise an insulin (preproinsulin, proinsulin, insulin) coding sequence. Preproinsulin, 110 amino acids in length, is a biologically inactive precursor to insulin. Insulin mRNA is translated as preproinsulin, a single chain precursor, and removal of its signal peptide during insertion into the endoplasmic reticulum generates proinsulin. Insulin is produced in and secreted by beta cells in the pancreas. Proinsulin and preproinsulin comprise three domains: an amino-terminal B-chain, a carboxy-terminal A-chain, and a connecting peptide in the middle known as the C-peptide. Within the endoplasmic reticulum, proinsulin is exposed to several specific endopeptidases which excise the C-peptide, generating the mature form of insulin which consists of the A and B-chain. Insulin and free C-peptide are packaged in the Golgi into secretory granules which accumulate in the cytoplasm. In some embodiments, the insulin protein comprises the amino acid sequence of SEQ ID NO: 46, or a variant thereof. It should be understood that unless otherwise stated, the term “insulin” encompasses the various forms of insulin, including preproinsulin, proinsulin, insulin.

In some embodiments, the glucoregulatory hormone coding sequence and the insulin coding sequence are arranged such that a functional glucoregulatory hormone and a functional insulin are produced in vivo following administration to a subject. As used herein, “functional” refers to a protein that possesses biological activity (e.g., enzymatic activity). That is, the functional protein produced has at least 10%, 20%, 30%, 40%, 50%, 60%, 70%, 80%, 90%, 100% or more activity (e.g., enzymatic activity) compared to a corresponding wild-type protein. Biological activity can be measured by any method known in the art; for example, by an in vitro activity assay or by in vivo measurements of enzymatic byproducts (e.g., C-peptide) or other related components (e.g., glucose levels). In some embodiments, the functional insulin has approximately the same activity as wild-type insulin (e.g., promotion of glucose uptake, glycogenesis, lipogenesis, and/or protein synthesis of skeletal muscle and/or fat tissue through the tyrosine kinase receptor pathway). In some embodiments, the functional insulin has more activity than wild-type insulin. In some embodiments, the functional glucoregulatory hormone has approximately the same activity as a corresponding wild-type glucoregulatory hormone (e.g., maintenance of circulating glucose concentrations within a physiological range). In some embodiments, the functional glucoregulatory hormone has more activity than a corresponding wild-type glucoregulatory hormone.

In some embodiments, the glucoregulatory coding sequence is nested within the insulin coding sequence; that is, the glucoregulatory coding sequence is located between the 5′ and 3′ ends of the insulin coding sequence. As used herein, “nested” refers to the positional relationship between the two coding sequences: one is located within the other and arranged such that each sequence is in frame, resulting in the production of insulin and the glucoregulatory hormone (e.g., GLP-1) in vivo. That is, nesting does not cause one or more frameshift mutations.

The glucoregulatory hormone coding sequence may be nested within or next to any of the insulin protein coding sequences. The insulin protein coding sequence comprises, from 5′ to 3′: the B-chain coding sequence, the C-peptide coding sequence, and the A-chain coding sequence. Therefore, in some embodiments, the glucoregulatory hormone coding sequence is nested within the C-peptide coding sequence. In some embodiments, the glucoregulatory hormone coding sequence is nested between the B-chain coding sequence and the C-peptide coding sequence. In some embodiments, the glucoregulatory hormone coding sequence is nested between the C-peptide coding sequence and the A-chain coding sequence.

In some embodiments, the glucoregulatory hormone coding sequence is flanked by one or more sequences, such as an intervening peptide sequence or a cleavage/enzyme processing site. As used herein, “flanked” refers to the addition of a polynucleotide sequence that is adjacent to (e.g., within 5, 10, 15, 20, 25, or 30 nucleotides of) the 5′ and/or 3′ end of the coding sequence. In some embodiments, the glucoregulatory hormone coding sequence is flanked at the 5′ end of the glucoregulatory hormone coding sequence. In some embodiments, the glucoregulatory hormone coding sequence is flanked at the 3′ end of the glucoregulatory hormone coding sequence. In some embodiments, the glucoregulatory hormone coding sequence is flanked at the 3′ end and the 5′ end of the glucoregulatory hormone coding sequence.

In some embodiments, a flanking sequence comprises a processing or cleavage signal sequence such that the resulting glucoregulatory hormone is cleaved from the resulting insulin. In some embodiments, a flanking sequence comprises a PCSK1 (also known as prohormone convertase 1/3 (PC1/3)) processing sequence and/or PCSK2 (also known as prohormone convertase 2 (PC2)) enzyme processing sequence. PCSK1 and PCSK2 are enzymes that act together to process proinsulin. PCSK1 cleaves the PCSK1 sequence (typically either a lysine-arginine (KR) or an arginine-arginine (RR) amino acid sequence), while PCSK2 cleaves the PCSK2 sequence (also either a KR or an RR amino acid sequence). It is not well understood what additional flanking amino acid sequences surround a given “KR” or “RR” enzyme processing site, or what additional considerations such as protein co-factors or secondary or tertiary protein structures confer additional specificity for either PCSK1 or PCSK2 for a given “KR” or “RR” enzyme processing site, and some redundancy certainly exists. Therefore, without wishing to be bound by theory, it is thought that the inclusion of one or two PCSK1 and/or PCSK2 enzyme processing sequences flanking the glucoregulatory hormone sequence leads to the excision of the glucoregulatory hormone from the proinsulin.

Other cleavage sites are contemplated herein, for example, furin cleavage sites (e.g., RXXR, RXKR, and RXRR).

In some embodiments, the glucoregulatory hormone coding sequence is flanked by a first PCSK1 and/or PCSK2 enzyme processing sequence (either on the 5′ end or the 3′ end). In other embodiments, the glucoregulatory hormone coding sequence is flanked by a first PCSK1 and/or PCSK2 enzyme processing sequence and a second PCSK1 and/or PCSK2 enzyme processing sequence (there is a flanking sequence at the 5′ end and at the 3′ end of the glucoregulatory hormone coding sequence). In some embodiments, a PCSK1 and/or PCSK2 enzyme processing sequence is a native (wild-type) sequence. In other embodiments, a PCSK1 and/or PCSK2 enzyme processing sequence is an artificial (engineered) sequence. As used herein, an “artificial sequence” is a nucleotide sequence (or amino acid sequence) that does not occur in nature (e.g., a polynucleotide without 100% identity with a naturally-occurring protein or a fragment thereof). In configurations or arrangements comprising two flanking PCSK1 and/or PCSK2 enzyme processing sequences, both PCSK1 and/or PCSK2 enzyme processing sequences may be artificial, both may be native, or one may be artificial while the other is native.

In some embodiments, one or both of the flanking sequences may further comprise a preproglucagon intervening peptide (IP) sequence (e.g., IP-1 or IP-2). In native (wild-type) proglucagon, GLP-1 is flanked by two IP sequences: IP-1 (5′ end) and IP-2 (3′ end). In some embodiments, the glucoregulatory hormone coding sequence is flanked by a first IP sequence (either on the 5′ end or the 3′ end). In other embodiments, the glucoregulatory hormone coding sequence is flanked by a first IP sequence and a second IP sequence (there is a flanking sequence at the 5′ end and at the 3′ end of the glucoregulatory hormone coding sequence). In some embodiments, an IP sequence is a native (wild-type) sequence (e.g., IP-1 or IP-2). In other embodiments, an IP sequence is an artificial (engineered) sequence. In configurations or arrangements comprising two flanking IP sequences, both IP sequences may be artificial, both may be native, or one may be artificial while the other is native.

In some embodiments, the glucoregulatory hormone coding sequence is flanked by an IP sequence and a processing sequence (e.g., PCSK1 and/or PCSK2 enzymatic processing sequence). For example, the glucoregulatory hormone coding sequence is flanked by PCSK1 and/or PCSK2 enzymatic processing sequences on both termini, and each PCSK1 and/or PCSK2 enzymatic processing sequence is flanked by an IP sequence. That is, from 5′ to 3′, the polynucleotide comprises a first IP sequence (e.g., encoding IP-1), a first PCSK1 and/or PCSK2 enzymatic processing sequence, the glucoregulatory hormone coding sequence, a second PCSK1 and/or PCSK2 enzymatic processing sequence, and a second IP sequence (e.g., encoding IP-2).

Different configurations or arrangements including the flanking sequence or sequences within the polynucleotide are possible. In some embodiments, the polynucleotide comprises, from 5′ to 3′, a signal peptide coding sequence, a B-chain coding sequence, a first PCSK1 and/or PCSK2 enzyme processing site, a C-peptide coding sequence, a glucoregulatory hormone coding sequence, a second PCSK1 and/or PCSK2 enzyme processing site, and an A-chain coding sequence. In another embodiment, the polynucleotide comprises, from 5′ to 3′, a signal peptide coding sequence, a B-chain coding sequence, a first PCSK1 and/or PCSK2 enzyme processing site, a C-peptide coding sequence, a second PCSK1 and/or PCSK2 enzyme processing site, a first preproglucagon IP sequence, a third PCSK1 and/or PCSK2 enzyme processing site, a glucoregulatory hormone coding sequence, a fourth PCSK1 and/or PCSK2 enzyme processing site, a second preproglucagon IP, a fifth PCSK1 and/or PCSK2 enzyme processing site, and an A-chain coding sequence.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular wild-type, native, or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm.

In some embodiments, the polynucleotides provided herein further comprise at least one promoter sequence. As used herein, a “promoter sequence” is a nucleotide sequence located at the 5′ terminal of the polynucleotide to which a polymerase specifically binds and initiates transcription of the remainder of the polynucleotide. In some embodiments, the promoter is a pancreatic islet cell promoter sequence, such as a pancreatic islet beta cell promoter sequence. In some embodiments, the promoter sequence is an insulin promoter sequence, such as a human insulin promoter, mouse insulin1 promoter, mouse insulin2 promoter, rat insulin2 promoter, or rat insulin1 promoter. Additional exemplary promoters include, but are not limited to, Slc2a, IAPP, NKX6.1, DLK1, MafA, Slc30a8/Znt8, PCSK1, and ADCYAP1.

In some embodiments, the polynucleotides provided herein further comprise an enhancer sequence. An “enhancer sequence” is a nucleotide sequence that can stimulate promoter activity by enhancing the level of tissue specificity of a promoter and is positioned between the promoter and the coding sequences of the polynucleotide. Exemplary enhancer sequences include, but are not limited to, a CMV enhancer, a synthetic enhancer, a liver-specific enhancer, a vascular-specific enhancer, a brain-specific enhancer, a neural cell-specific enhancer, a lung-specific enhancer, a muscle-specific enhancer, a kidney-specific enhancer, a pancreas-specific enhancer, and an islet cell-specific enhancer. In some embodiments, the enhancer sequence is an islet cell-specific enhancer.

In some embodiments, the polynucleotide comprises a nucleotide sequence having at least 80%, at least 85%, at least 90%, at least 95%, or 100% identity to the nucleotide sequence of SEQ ID NO: 59. In some embodiments, the polynucleotide comprises a nucleotide sequence having at least 80%, at least 85%, at least 90%, at least 95%, or 100% identity to the nucleotide sequence of SEQ ID NO: 60. In some embodiments, the polynucleotide comprises a nucleotide sequence having at least 80%, at least 85%, at least 90%, at least 95%, or 100% identity to the nucleotide sequence of SEQ ID NO: 61. In some embodiments, the polynucleotide comprises a nucleotide sequence having at least 80%, at least 85%, at least 90%, at least 95%, or 100% identity to the nucleotide sequence of SEQ ID NO: 62. In some embodiments, the polynucleotide comprises a nucleotide sequence having at least 80%, at least 85%, at least 90%, at least 95%, or 100% identity to the nucleotide sequence of SEQ ID NO: 63. In some embodiments, the polynucleotide comprises a nucleotide sequence having at least 80%, at least 85%, at least 90%, at least 95%, or 100% identity to the nucleotide sequence of SEQ ID NO: 64. In some embodiments, the polynucleotide comprises a nucleotide sequence having at least 80%, at least 85%, at least 90%, at least 95%, or 100% identity to the nucleotide sequence of SEQ ID NO: 65. In some embodiments, the polynucleotide comprises a nucleotide sequence having at least 80%, at least 85%, at least 90%, at least 95%, or 100% identity to the nucleotide sequence of SEQ ID NO: 66. In some embodiments, the polynucleotide comprises a nucleotide sequence having at least 80%, at least 85%, at least 90%, at least 95%, or 100% identity to the nucleotide sequence of SEQ ID NO: 67. In some embodiments, the polynucleotide comprises a nucleotide sequence having at least 80%, at least 85%, at least 90%, at least 95%, or 100% identity to the nucleotide sequence of SEQ ID NO: 68. In some embodiments, the polynucleotide comprises a nucleotide sequence having at least 80%, at least 85%, at least 90%, at least 95%, or 100% identity to the nucleotide sequence of SEQ ID NO: 69. In some embodiments, the polynucleotide comprises a nucleotide sequence having at least 80%, at least 85%, at least 90%, at least 95%, or 100% identity to the nucleotide sequence of SEQ ID NO: 70. In some embodiments, the polynucleotide comprises a nucleotide sequence having at least 80%, at least 85%, at least 90%, at least 95%, or 100% identity to the nucleotide sequence of SEQ ID NO: 71. In some embodiments, the polynucleotide comprises a nucleotide sequence having at least 80%, at least 85%, at least 90%, at least 95%, or 100% identity to the nucleotide sequence of SEQ ID NO: 85. In some embodiments, the polynucleotide comprises a nucleotide sequence having at least 80%, at least 85%, at least 90%, at least 95%, or 100% identity to the nucleotide sequence of SEQ ID NO: 86. In some embodiments, the polynucleotide comprises a nucleotide sequence having at least 80%, at least 85%, at least 90%, at least 95%, or 100% identity to the nucleotide sequence of SEQ ID NO: 87.

In some embodiments, the polynucleotide encodes an amino acid sequence having at least 80%, at least 85%, at least 90%, at least 95%, or 100% identity to the amino acid sequence of SEQ ID NO: 72. In some embodiments, the polynucleotide encodes an amino acid sequence having at least 80%, at least 85%, at least 90%, at least 95%, or 100% identity to the amino acid sequence of SEQ ID NO: 73. In some embodiments, the polynucleotide encodes an amino acid sequence having at least 80%, at least 85%, at least 90%, at least 95%, or 100% identity to the amino acid sequence of SEQ ID NO: 74. In some embodiments, the polynucleotide encodes an amino acid sequence having at least 80%, at least 85%, at least 90%, at least 95%, or 100% identity to the amino acid sequence of SEQ ID NO: 75. In some embodiments, the polynucleotide encodes an amino acid sequence having at least 80%, at least 85%, at least 90%, at least 95%, or 100% identity to the amino acid sequence of SEQ ID NO: 76. In some embodiments, the polynucleotide encodes an amino acid sequence having at least 80%, at least 85%, at least 90%, at least 95%, or 100% identity to the amino acid sequence of SEQ ID NO: 77. In some embodiments, the polynucleotide encodes an amino acid sequence having at least 80%, at least 85%, at least 90%, at least 95%, or 100% identity to the amino acid sequence of SEQ ID NO: 78. In some embodiments, the polynucleotide encodes an amino acid sequence having at least 80%, at least 85%, at least 90%, at least 95%, or 100% identity to the amino acid sequence of SEQ ID NO: 79. In some embodiments, the polynucleotide encodes an amino acid sequence having at least 80%, at least 85%, at least 90%, at least 95%, or 100% identity to the amino acid sequence of SEQ ID NO: 80. In some embodiments, the polynucleotide encodes an amino acid sequence having at least 80%, at least 85%, at least 90%, at least 95%, or 100% identity to the amino acid sequence of SEQ ID NO: 81. In some embodiments, the polynucleotide encodes an amino acid sequence having at least 80%, at least 85%, at least 90%, at least 95%, or 100% identity to the amino acid sequence of SEQ ID NO: 82. In some embodiments, the polynucleotide encodes an amino acid sequence having at least 80%, at least 85%, at least 90%, at least 95%, or 100% identity to the amino acid sequence of SEQ ID NO: 83. In some embodiments, the polynucleotide encodes an amino acid sequence having at least 80%, at least 85%, at least 90%, at least 95%, or 100% identity to the amino acid sequence of SEQ ID NO: 84. In some embodiments, the polynucleotide encodes an amino acid sequence having at least 80%, at least 85%, at least 90%, at least 95%, or 100% identity to the amino acid sequence of SEQ ID NO: 88 In some embodiments, the polynucleotide encodes an amino acid sequence having at least 80%, at least 85%, at least 90%, at least 95%, or 100% identity to the amino acid sequence of SEQ ID NO: 89. In some embodiments, the polynucleotide encodes an amino acid sequence having at least 80%, at least 85%, at least 90%, at least 95%, or 100% identity to the amino acid sequence of SEQ ID NO: 90.

TABLE 2
Examples of Fusion Protein Coding Sequences and Protein Sequences

| Name | Nucleic Acid | SEQ ID NO: |
|---|---|---|
| INS-GLP-1 Hybrid | ATGGCCCTGTGGATGCGCCTCCTGCCCCTGCTGGCGCTGCTGGCCCT | 59 |
| #01 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGAGGAACAGGAATAACATT | |
| | GCCAAACGTCACGATGAATTTGAGAGACATGCTGAAGGGACCTTTAC | |
| | CAGTGATGTAAGTTCTTATTTGGAAGGCCAAGCTGCCAAGGAATTCA | |
| | TTGCTTGGCTGGTGAAAGGCCGAGGAAGGCGAGATTTCCCAAAGCGT | |
| | GGCATTGTGGAACAATGCTGTACCAGCATCTGCTCCCTCTACCAGCT | |
| | GGAGAACTACTGCAACTAG | |
| INS-GLP-1 Hybrid | ATGGCCCTGTGGATGCGCCTCCTGCCCCTGCTGGCGCTGCTGGCCCT | 60 |
| #02 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGAGGAACAGGAATAACATT | |
| | GCCAAACGTCACGATGAATTTGAGAGACATGCTGAAGGGACCTTTAC | |
| | CAGTGATGTAAGTTCTTATTTGGAAGGCCAAGCTGCCAAGGAATTCA | |
| | TTGCTTGGCTGGTGAAAGGCCGAGGAAGGCGAGATTTCCCAGAAGAG | |
| | GTCGCCATTGTTGAAGAACTTGGCCGCAGAAAGCGTGGCATTGTGGA | |
| | ACAATGCTGTACCAGCATCTGCTCCCTCTACCAGCTGGAGAACTACT | |
| | GCAACTAG | |
| INS-GLP-1 Hybrid | ATGGCCCTGTGGATGCGCCTCCTGCCCCTGCTGGCGCTGCTGGCCCT | 61 |
| #03 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCAAGAGGAACAGGAATAACATTGCCAA | |
| | ACGTCACGATGAATTTGAGAGACATGCTGAAGGGACCTTTACCAGTG | |
| | ATGTAAGTTCTTATTTGGAAGGCCAAGCTGCCAAGGAATTCATTGCT | |
| | TGGCTGGTGAAAGGCCGAGGAAGGCGAGATTTCCCAGAAGAGGTCGC | |
| | CATTGTTGAAGAACTTGGCCGCAGACGCCGGGAGGCAGAGGACCTGC | |
| | AGGTGGGGCAGGTGGAGCTGGGCGGGGGCCCTGGTGCAGGCAGCCTG | |
| | CAGCCCTTGGCCCTGGAGGGGTCCCTGCAGAAGCGTGGCATTGTGGA | |
| | ACAATGCTGTACCAGCATCTGCTCCCTCTACCAGCTGGAGAACTACT | |
| | GCAACTAG | |
| INS-GLP-1 Hybrid | ATGGCCCTGTGGATGCGCCTCCTGCCCCTGCTGGCGCTGCTGGCCCT | 62 |
| #04 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCAAGAGGAACAGGAATAACATTG | |
| | CCAAACGTCACGATGAATTTGAGAGACATGCTGAAGGGACCTTTACC | |
| | AGTGATGTAAGTTCTTATTTGGAAGGCCAAGCTGCCAAGGAATTCAT | |
| | TGCTTGGCTGGTGAAAGGCCGAGGAAGGCGAGATTTCCCAGAAGAGG | |
| | TCGCCATTGTTGAAGAACTTGGCCGCAGACCTGGTGCAGGCAGCCTG | |
| | CAGCCCTTGGCCCTGGAGGGGTCCCTGCAGAAGCGTGGCATTGTGGA | |
| | ACAATGCTGTACCAGCATCTGCTCCCTCTACCAGCTGGAGAACTACT | |
| | GCAACTAG | |
| INS-GLP-1 Hybrid | ATGGCCCTGTGGATGCGCCTCCTGCCCCTGCTGGCGCTGCTGGCCCT | 63 |
| #05 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAACGTCACGATGAATTTGAG | |
| | AGACATGCTGAAGGGACCTTTACCAGTGATGTAAGTTCTTATTTGGA | |
| | AGGCCAAGCTGCCAAGGAATTCATTGCTTGGCTGGTGAAAGGCCGAG | |
| | GAAGGCGAGATTTCCCAGAAGAGGTCGCCATTGTTGAAGAACTTGGC | |
| | CGCAGAAAGCGTGGCATTGTGGAACAATGCTGTACCAGCATCTGCTC | |
| | CCTCTACCAGCTGGAGAACTACTGCAACTAG | |
| INS-GLP-1 Hybrid | ATGGCCCTGTGGATGCGCCTCCTGCCCCTGCTGGCGCTGCTGGCCCT | 64 |
| #06 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGAGGAACAGGAATAACATT | |
| | GCCAAACGTCACGATGAATTTGAGAGACATGCTGAAGGGACCTTTAC | |
| | CAGTGATGTAAGTTCTTATTTGGAAGGCCAAGCTGCCAAGGAATTCA | |
| | TTGCTTGGCTGGTGAAAGGCCGAGGAAGGCGAAAGCGTGGCATTGTG | |
| | GAACAATGCTGTACCAGCATCTGCTCCCTCTACCAGCTGGAGAACTA | |
| | CTGCAACTAG | |
| INS-GLP-1 Hybrid | ATGGCCCTGTGGATGCGCCTCCTGCCCCTGCTGGCGCTGCTGGCCCT | 65 |
| #07 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCAAACGTCACGATGAATTTGAGAGACA | |
| | TGCTGAAGGGACCTTTACCAGTGATGTAAGTTCTTATTTGGAAGGCC | |
| | AAGCTGCCAAGGAATTCATTGCTTGGCTGGTGAAAGGCCGAGGAAGG | |
| | CGACGCCGGGAGGCAGAGGACCTGCAGGTGGGGCAGGTGGAGCTGGG | |
| | CGGGGGCCCTGGTGCAGGCAGCCTGCAGCCCTTGGCCCTGGAGGGGT | |
| | CCCTGCAGAAGCGTGGCATTGTGGAACAATGCTGTACCAGCATCTGC | |
| | TCCCTCTACCAGCTGGAGAACTACTGCAACTAG | |
| INS-GLP-1 Hybrid | ATGGCCCTGTGGATGCGCCTCCTGCCCCTGCTGGCGCTGCTGGCCCT | 66 |
| #08 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCAAACGTCACGATGAATTTGAGA | |
| | GACATGCTGAAGGGACCTTTACCAGTGATGTAAGTTCTTATTTGGAA | |
| | GGCCAAGCTGCCAAGGAATTCATTGCTTGGCTGGTGAAAGGCCGAGG | |
| | AAGGCGACCTGGTGCAGGCAGCCTGCAGCCCTTGGCCCTGGAGGGGT | |
| | CCCTGCAGAAGCGTGGCATTGTGGAACAATGCTGTACCAGCATCTGC | |
| | TCCCTCTACCAGCTGGAGAACTACTGCAACTAG | |
| INS-GLP-1 Hybrid | ATGGCCCTGTGGATGCGCCTCCTGCCCCTGCTGGCGCTGCTGGCCCT | 67 |
| #09 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAACGTCACGATGAATTTGAG | |
| | AGACATGCTGAAGGGACCTTTACCAGTGATGTAAGTTCTTATTTGGA | |
| | AGGCCAAGCTGCCAAGGAATTCATTGCTTGGCTGGTGAAAGGCCGAG | |
| | GAAGGCGAAAGCGTGGCATTGTGGAACAATGCTGTACCAGCATCTGC | |
| | TCCCTCTACCAGCTGGAGAACTACTGCAACTAG | |
| INS-GLP-1 Hybrid | ATGGCCCTGTGGATGCGCCTCCTGCCCCTGCTGGCGCTGCTGGCCCT | 68 |
| #10 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGCGTGGCATTGTGGAACAA | |
| | TGCTGTACCAGCATCTGCTCCCTCTACCAGCTGGAGAACTACTGCAA | |
| | CAAGAGGAACAGGAATAACATTGCCAAACGTCACGATGAATTTGAGA | |
| | GACATGCTGAAGGGACCTTTACCAGTGATGTAAGTTCTTATTTGGAA | |
| | GGCCAAGCTGCCAAGGAATTCATTGCTTGGCTGGTGAAAGGCCGAGG | |
| | AAGGCGAGATTTCCCAGAAGAGGTCGCCATTGTTGAAGAACTTGGCC | |
| | GCAGATAG | |
| INS-GLP-1 Hybrid | ATGGCCCTGTGGATGCGCCTCCTGCCCCTGCTGGCGCTGCTGGCCCT | 69 |
| #11 | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGCGTGGCATTGTGGAACAA | |
| | TGCTGTACCAGCATCTGCTCCCTCTACCAGCTGGAGAACTACTGCAA | |
| | CAAACGTCACGATGAATTTGAGAGACATGCTGAAGGGACCTTTACCA | |
| | GTGATGTAAGTTCTTATTTGGAAGGCCAAGCTGCCAAGGAATTCATT | |
| | GCTTGGCTGGTGAAAGGCCGAGGAAGGCGATAG | |
| INS-GLP-1 Hybrid | ATGGCCCTGTGGATGCGCCTCCTGCCCCTGCTGGCGCTGCTGGCCCT | 70 |
| #12 | CTGGGGACCTGACCCAGCCGCAGCCAAGAGGAACAGGAATAACATTG | |
| | CCAAACGTCACGATGAATTTGAGAGACATGCTGAAGGGACCTTTACC | |
| | AGTGATGTAAGTTCTTATTTGGAAGGCCAAGCTGCCAAGGAATTCAT | |
| | TGCTTGGCTGGTGAAAGGCCGAGGAAGGCGAGATTTCCCAGAAGAGG | |
| | TCGCCATTGTTGAAGAACTTGGCCGCAGATTTGTGAACCAACACCTG | |
| | TGCGGCTCACACCTGGTGGAAGCTCTCTACCTAGTGTGCGGGGAACG | |
| | AGGCTTCTTCTACACACCCAAGACCCGCCGGGAGGCAGAGGACCTGC | |
| | AGGTGGGGCAGGTGGAGCTGGGCGGGGGCCCTGGTGCAGGCAGCCTG | |
| | CAGCCCTTGGCCCTGGAGGGGTCCCTGCAGAAGCGTGGCATTGTGGA | |
| | ACAATGCTGTACCAGCATCTGCTCCCTCTACCAGCTGGAGAACTACT | |
| | GCAACTAG | |
| INS-GLP-1 Hybrid | ATGGCCCTGTGGATGCGCCTCCTGCCCCTGCTGGCGCTGCTGGCCCT | 71 |
| #13 | CTGGGGACCTGACCCAGCCGCAGCCAAACGTCACGATGAATTTGAGA | |
| | GACATGCTGAAGGGACCTTTACCAGTGATGTAAGTTCTTATTTGGAA | |
| | GGCCAAGCTGCCAAGGAATTCATTGCTTGGCTGGTGAAAGGCCGAGG | |
| | AAGGCGATTTGTGAACCAACACCTGTGCGGCTCACACCTGGTGGAAG | |
| | CTCTCTACCTAGTGTGCGGGGAACGAGGCTTCTTCTACACACCCAAG | |
| | ACCCGCCGGGAGGCAGAGGACCTGCAGGTGGGGCAGGTGGAGCTGGG | |
| | CGGGGGCCCTGGTGCAGGCAGCCTGCAGCCCTTGGCCCTGGAGGGGT | |
| | CCCTGCAGAAGCGTGGCATTGTGGAACAATGCTGTACCAGCATCTGC | |
| | TCCCTCTACCAGCTGGAGAACTACTGCAACTAG | |
| INS-PYY Hybrid #01 | ATGGCCCTGTGGATGCGCCTCCTGCCCCTGCTGGCGCTGCTGGCCCT | 85 |
| | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGTACCCCATCAAACCCGAGGC | |
| | TCCCCGCGAAGACGCCTCGCCGGAGGAGCTGAACCGCTACTACGCCT | |
| | CCCTGCGCCACTACCTCAACCTGGTCACCCGGCAGCGGTATGGGAAA | |
| | AGAGAGGCAGAGGACCTGCAGGTGGGGCAGGTGGAGCTGGGCGGGGG | |
| | CCCTGGTGCAGGCAGCCTGCAGCCCTTGGCCCTGGAGGGGTCCCTGC | |
| | AGAAGCGTGGCATTGTGGAACAATGCTGTACCAGCATCTGCTCCCTC | |
| | TACCAGCTGGAGAACTACTGCAACTAG | |
| INS-PYY Hybrid #02 | ATGGCCCTGTGGATGCGCCTCCTGCCCCTGCTGGCGCTGCTGGCCCT | 86 |
| | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGCGTTACCCCATCAAACCC | |
| | GAGGCTCCCCGCGAAGACGCCTCGCCGGAGGAGCTGAACCGCTACTA | |
| | CGCCTCCCTGCGCCACTACCTCAACCTGGTCACCCGGCAGCGGTATG | |
| | GGAAAAGAGGCATTGTGGAACAATGCTGTACCAGCATCTGCTCCCTC | |
| | TACCAGCTGGAGAACTACTGCAACTAG | |
| INS-PYY Hybrid #03 | ATGGCCCTGTGGATGCGCCTCCTGCCCCTGCTGGCGCTGCTGGCCCT | 87 |
| | CTGGGGACCTGACCCAGCCGCAGCCTTTGTGAACCAACACCTGTGCG | |
| | GCTCACACCTGGTGGAAGCTCTCTACCTAGTGTGCGGGGAACGAGGC | |
| | TTCTTCTACACACCCAAGACCCGCCGGGAGGCAGAGGACCTGCAGGT | |
| | GGGGCAGGTGGAGCTGGGCGGGGGCCCTGGTGCAGGCAGCCTGCAGC | |
| | CCTTGGCCCTGGAGGGGTCCCTGCAGAAGCGTGGCATTGTGGAACAA | |
| | TGCTGTACCAGCATCTGCTCCCTCTACCAGCTGGAGAACTACTGCAA | |
| | CAAGCGTTACCCCATCAAACCCGAGGCTCCCCGCGAAGACGCCTCGC | |
| | CGGAGGAGCTGAACCGCTACTACGCCTCCCTGCGCCACTACCTCAAC | |
| | CTGGTCACCCGGCAGCGGTATGGGAAAAGATAG | |
| Name | Amino Acid | SEQ ID NO: |
| INS-GLP-1 Hybrid | MALWMRLLPLLALLALWGPDPAAAFVNQHLCGSHLVEALYLVCGERG | 72 |
| #01 | FFYTPKTRREAEDLQVGQVELGGGPGAGSLQPLALEGSLQKRNRNNI | |
| | AKRHDEFERHAEGTFTSDVSSYLEGQAAKEFIAWLVKGRGRRDFPKR | |
| | GIVEQCCTSICSLYQLENYCN | |
| INS-GLP-1 Hybrid | MALWMRLLPLLALLALWGPDPAAAFVNQHLCGSHLVEALYLVCGERG | 73 |
| #02 | FFYTPKTRREAEDLQVGQVELGGGPGAGSLQPLALEGSLQKRNRNNI | |
| | AKRHDEFERHAEGTFTSDVSSYLEGQAAKEFIAWLVKGRGRRDFPEE | |
| | VAIVEELGRRKRGIVEQCCTSICSLYQLENYCN | |
| INS-GLP-1 Hybrid | MALWMRLLPLLALLALWGPDPAAAFVNQHLCGSHLVEALYLVCGERG | 74 |
| #03 | FFYTPKTKRNRNNIAKRHDEFERHAEGTFTSDVSSYLEGQAAKEFIA | |
| | WLVKGRGRRDFPEEVAIVEELGRRRREAEDLQVGQVELGGGPGAGSL | |
| | QPLALEGSLQKRGIVEQCCTSICSLYQLENYCN | |
| INS-GLP-1 Hybrid | MALWMRLLPLLALLALWGPDPAAAFVNQHLCGSHLVEALYLVCGERG | 75 |
| #04 | FFYTPKTRREAEDLQVGQVELGGGKRNRNNIAKRHDEFERHAEGTFT | |
| | SDVSSYLEGQAAKEFIAWLVKGRGRRDFPEEVAIVEELGRRPGAGSL | |
| | QPLALEGSLQKRGIVEQCCTSICSLYQLENYCN | |
| INS-GLP-1 Hybrid | MALWMRLLPLLALLALWGPDPAAAFVNQHLCGSHLVEALYLVCGERG | 76 |
| #05 | FFYTPKTRREAEDLQVGQVELGGGPGAGSLQPLALEGSLQKRHDEFE | |
| | RHAEGTFTSDVSSYLEGQAAKEFIAWLVKGRGRRDFPEEVAIVEELG | |
| | RRKRGIVEQCCTSICSLYQLENYCN | |
| INS-GLP-1 Hybrid | MALWMRLLPLLALLALWGPDPAAAFVNQHLCGSHLVEALYLVCGERG | 77 |
| #06 | FFYTPKTRREAEDLQVGQVELGGGPGAGSLQPLALEGSLQKRNRNNI | |
| | AKRHDEFERHAEGTFTSDVSSYLEGQAAKEFIAWLVKGRGRRKRGIV | |
| | EQCCTSICSLYQLENYCN | |
| INS-GLP-1 Hybrid | MALWMRLLPLLALLALWGPDPAAAFVNQHLCGSHLVEALYLVCGERG | 78 |
| #07 | FFYTPKTKRHDEFERHAEGTFTSDVSSYLEGQAAKEFIAWLVKGRGR | |
| | RRREAEDLQVGQVELGGGPGAGSLQPLALEGSLQKRGIVEQCCTSIC | |
| | SLYQLENYCN | |
| INS-GLP-1 Hybrid | MALWMRLLPLLALLALWGPDPAAAFVNQHLCGSHLVEALYLVCGERG | 79 |
| #08 | FFYTPKTRREAEDLQVGQVELGGGKRHDEFERHAEGTFTSDVSSYLE | |
| | GQAAKEFIAWLVKGRGRRPGAGSLQPLALEGSLQKRGIVEQCCTSIC | |
| | SLYQLENYCN | |
| INS-GLP-1 Hybrid | MALWMRLLPLLALLALWGPDPAAAFVNQHLCGSHLVEALYLVCGERG | 80 |
| #09 | FFYTPKTRREAEDLQVGQVELGGGPGAGSLQPLALEGSLQKRHDEFE | |
| | RHAEGTFTSDVSSYLEGQAAKEFIAWLVKGRGRRKRGIVEQCCTSIC | |
| | SLYQLENYCN | |
| INS-GLP-1 Hybrid | MALWMRLLPLLALLALWGPDPAAAFVNQHLCGSHLVEALYLVCGERG | 81 |
| #10 | FFYTPKTRREAEDLQVGQVELGGGPGAGSLQPLALEGSLQKRGIVEQ | |
| | CCTSICSLYQLENYCNKRNRNNIAKRHDEFERHAEGTFTSDVSSYLE | |
| | GQAAKEFIAWLVKGRGRRDFPEEVAIVEELGRR | |
| INS-GLP-1 Hybrid | MALWMRLLPLLALLALWGPDPAAAFVNQHLCGSHLVEALYLVCGERG | 82 |
| #11 | FFYTPKTRREAEDLQVGQVELGGGPGAGSLQPLALEGSLQKRGIVEQ | |
| | CCTSICSLYQLENYCNKRHDEFERHAEGTFTSDVSSYLEGQAAKEFI | |
| | AWLVKGRGRR | |
| INS-GLP-1 Hybrid | MALWMRLLPLLALLALWGPDPAAAKRNRNNIAKRHDEFERHAEGTFT | 83 |
| #12 | SDVSSYLEGQAAKEFIAWLVKGRGRRDFPEEVAIVEELGRRFVNQHL | |
| | CGSHLVEALYLVCGERGFFYTPKTRREAEDLQVGQVELGGGPGAGSL | |
| | QPLALEGSLQKRGIVEQCCTSICSLYQLENYCN | |
| INS-GLP-1 Hybrid | MALWMRLLPLLALLALWGPDPAAAKRHDEFERHAEGTFTSDVSSYLE | 84 |
| #13 | GQAAKEFIAWLVKGRGRRFVNQHLCGSHLVEALYLVCGERGFFYTPK | |
| | TRREAEDLQVGQVELGGGPGAGSLQPLALEGSLQKRGIVEQCCTSIC | |
| | SLYQLENYCN | |
| INS-PYY Hybrid #01 | MALWMRLLPLLALLALWGPDPAAAFVNQHLCGSHLVEALYLVCGERG | 88 |
| | FFYTPKTRRYPIKPEAPREDASPEELNRYYASLRHYLNLVTRQRYGK | |
| | REAEDLQVGQVELGGGPGAGSLQPLALEGSLQKRGIVEQCCTSICSL | |
| | YQLENYCN | |
| INS-PYY Hybrid #02 | MALWMRLLPLLALLALWGPDPAAAFVNQHLCGSHLVEALYLVCGERG | 89 |
| | FFYTPKTRREAEDLQVGQVELGGGPGAGSLQPLALEGSLQKRYPIKP | |
| | EAPREDASPEELNRYYASLRHYLNLVTRQRYGKRGIVEQCCTSICSL | |
| | YQLENYCN | |
| INS-PYY Hybrid #03 | MALWMRLLPLLALLALWGPDPAAAFVNQHLCGSHLVEALYLVCGERG | 90 |
| | FFYTPKTRREAEDLQVGQVELGGGPGAGSLQPLALEGSLQKRGIVEQ | |
| | CCTSICSLYQLENYCNKRYPIKPEAPREDASPEELNRYYASLRHYLN | |
| | LVTRQRYGKR | |

### Delivery Systems and Routes

In some embodiments, the polynucleotides of the disclosure are further formatted for delivery. The disclosure, in some embodiments, provides vectors comprising any one of the polynucleotides described herein.

### Nonviral Vectors

In some embodiments, the vector is a nonviral vector, such as a plasmid, bacterial artificial chromosome, yeast artificial chromosome, or minicircle.

In some embodiments, the polynucleotides are delivered as plasmid vectors. In some embodiments, the polynucleotide is part of a nucleic acid cassette comprising the necessary elements for expression of the encoded polypeptide within the cassette. Thus, in some aspects, a plasmid is provided for expression of the encoded polypeptide which includes an expression cassette comprising the coding sequence for the polypeptide; also referred to as a transcription unit. When a plasmid is placed in an environment suitable for expression, the transcriptional unit will express the polypeptide and anything else encoded in the construct. The transcription unit includes a transcriptional control sequence, which is transcriptionally linked with a cellular immune response element coding sequence. Transcriptional control sequence may include promoter/enhancer sequences such as cytomegalovirus (CMV) promoter/enhancer sequences, such as described above. However, those skilled in the art will recognize that a variety of other promoter sequences suitable for expression in mammalian cells, including human patient cells, are known and can similarly be used in the constructs disclosed herein. The level of expression of the polypeptide will depend on the associated promoter and the presence and activation of an associated enhancer element.

In some embodiments, the polynucleotide can be cloned into an expression plasmid which contains the regulatory elements for transcription, translation, RNA stability, and replication (e.g., including a transcriptional control sequence). Such expression plasmids are well known in the art and one of ordinary skill would be capable of designing an appropriate expression construct for producing a recombinant polypeptide described herein in vivo.

In some embodiments, the polynucleotide can be formatted as a bacterial artificial chromosome (BAC). A BAC is an engineered DNA molecule used to clone DNA sequences in bacterial cells (e.g., E. coli). On average, DNA sequences ranging from 30,000 to about 300,000 base pairs can be inserted into BACs. BACs with inserted DNA can be taken up by bacterial cells. As bacterial cells grow and divide, the BAC DNA has very low bacterial cell copy number per bacterial cell (e.g., one copy per cell) and is maintained stably under specific conditions.

In some embodiments, the polynucleotide is formatted as a yeast artificial chromosome (YAC). A YAC is a genetically modified circular chromosome containing elements from a yeast chromosome (such as yeast) and foreign DNA (e.g., the polynucleotides described herein. YAC vectors contain specific structural components for replication in yeast, including, but not limited to: centromere, telomere, autonomously replicating sequence (ARS), yeast selectable markers (e.g., TRP1, URA3, and SUP4), and cloning sites for insertion of large segments of exogenous DNA over 50 kb.

In some embodiments, the polynucleotide is formatted as a minicircle. Minicircle (mcDNA)-based gene transfer can also be adapted for delivery of encoded polypeptides to tissues in vivo. As plasmid DNA can cause undesired inflammatory responses, minicircles, which do not comprise the same elements of plasmid DNA and are less immunogenic, are used in some embodiments. In minicircles, the immunogenic bacterial control regions, such as the origin of replication and antibiotic resistance genes, are eliminated from gene delivery vectors during the process of plasmid production. Thus, the “parent” plasmid is recombined into a “minicircle” which generally comprises the polypeptide to be delivered (in this case, the polynucleotide coding sequence) and suitable control regions for its expression. Therefore, embodiments of the polynucleotides described herein may be processed in the form of minicircle DNA. Minicircle DNA pertains to small (2-4 kb) circular plasmid derivatives that have been freed from all prokaryotic vector parts.

### Viral Vectors

In some embodiments, the vector is a viral vector, such as a retroviral vector, an adenovirus vector, a Herpes simplex virus (HSV) vector, and an adeno-associated virus (AAV) vector.

In some embodiments, the vector is a retroviral vector. The polynucleotide is inserted into the viral genome in the place of certain viral sequences to produce a replication-defective virus. To produce virions, a packaging cell line containing the gag, pol and env genes but without the LTR (long terminal repeat) and psi components is constructed (Mann et al., Cell, 33:153-159 (1983)). When a recombinant plasmid containing the polynucleotide, LTR and psi is introduced into this cell line, the psi sequence allows the RNA transcript of the recombinant plasmid to be packaged into viral particles, which are then secreted into the culture media. The media containing the recombinant retroviruses is then collected, optionally concentrated and used for gene delivery system.

In some embodiments, the retroviral vector for use in the present disclosure is a lentiviral vector, which refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV). In some embodiments, the retroviral vectors are those based upon murine leukemia virus (MuLV), gibbon ape leukemia virus (GaLV), and combinations thereof. Still other retroviral vectors that can also be used in the present disclosure include, e.g., vectors based on human foamy virus (HFV) or other viruses in the Spumavirus genera.

In some embodiments, a retroviral vector contains all of the cis-acting sequences necessary for the packaging and integration of the viral genome, e.g., (a) a long terminal repeat (LTR), or portions thereof, at each end of the vector; (b) primer binding sites for negative and positive strand DNA synthesis; and (c) a packaging signal, necessary for the incorporation of genomic RNA into virions. In some embodiments, the retrovirus is a recombinant replication competent retrovirus comprising a nucleotide sequence encoding a retroviral GAG protein; a nucleotide sequence encoding a retroviral POL protein; a nucleotide sequence encoding a retroviral envelope; an oncoretroviral polynucleotide sequence comprising Long-Terminal Repeat (LTR) sequences at the 5′ and 3′ end of the oncoretroviral polynucleotide sequence; a cassette comprising an internal ribosome entry site (IRES) operably linked to a polynucleotide described herein, wherein the cassette is positioned 5′ to the U3 region of the 3′ LTR and 3′ to the sequence encoding the retroviral envelope; and cis-acting sequences for reverse transcription, packaging and integration in a target cell (e.g., pancreatic beta cell).

In some embodiments, the vector is a herpes simplex virus (HSV) vector. HSV-based vectors are either replication defective viruses, whose cytotoxicity has been eliminated by deleting viral gene products, or amplicon vectors, which are plasmids packaged into HSV particles with the aid of a helper virus (Lachmann, Int J Exp Pathol, 2004; 85 (4): 177-190; Warnock et al. (2011) Methods Mol. Biol. 737:1-25). Herpes simplex virus (HSV) 1 and 2 are members of the Herpesviridae family and infect humans. The HSV genome contains two distinct areas designated as the unique long (UL) and unique short (US) regions. Each of these regions is flanked by a pair of inverted terminal repeat sequences and can be replaced with a polynucleotide described herein.

In some embodiments, the vector is an adenoviral vector (AdV). AdVs are non-enveloped, double-stranded DNA viruses that do not integrate in the host genome or replicate during cell division. An “adenovirus expression vector” refers to vectors comprising adenovirus sequences sufficient to (a) support packaging of the construct and (b) to express a polynucleotide described herein. Adenovirus has been usually employed as a gene delivery vector because of its mid-sized genome, ease of manipulation, high titer, wide target-cell range and high infectivity. Both ends of the viral genome contains 100-200 bp ITRs (inverted terminal repeats), which are cis elements necessary for viral DNA replication and packaging. The E1 region (E1A and E1B) of genome encodes proteins responsible for the regulation of transcription of the viral genome and a few cellular genes. The E2 region (E2A and E2B) encodes proteins responsible for viral DNA replication. In some embodiments, the polynucleotide sequence may be inserted into the DA promoter region.

In some embodiments, the vector comprises an adeno-associated virus (AAV) vector. AAVs (or “rAAV” for recombinant AAV) are non-enveloped small, single-stranded DNA viruses capable of infecting both dividing and non-dividing cells. In contrast to the generally limited durability of AdV-mediated gene transfer, transgene expression can persist for years following intramuscular recombinant AAV (rAAV) vector delivery.

Typically, a recombinant AAV virus is made by co-transfecting a plasmid containing the gene of interest (e.g., the polynucleotide) flanked by the two AAV terminal repeats and an expression plasmid containing the wild type AAV coding sequences without the terminal repeats. The AAV expression vector which harbors the polynucleotide bounded by AAV ITRs, can be constructed by directly inserting the selected sequence(s) into an AAV genome which has had the major AAV open reading frames (“ORFs”) excised therefrom. In some embodiments, the ITR sequences are selected from AAV1, AAV2, AAV3, AAV4, AAV5, AAV6, AAV7, AAV8, AAV9, AAV10 (AAVrh10), and AAV11 ITR sequences. In some embodiments, the ITRs are designed for a single-stranded AAV genome or a self-complimentary AAV genome. With respect to the self-complimentary AAV genome, the TRS (terminal resolution site) located in the 3′ ITR is deleted.

In some embodiments, the AAV vector comprises the recombinant AAV vector genome described above and a nucleotide sequence encoding a capsid protein. Capsid proteins are related to the determination of the tissue-specific targeting capabilities of an AAV and are known in the art. In some embodiments, the capsid protein is selected from AAV1, AAV2, AAV3, AAV4, AAV5, AAV6, AAV7, AAV8, AAV9, AAV10, AAV11, Rh10, Rh74, AAV-218, AAV-DJ, AAV-LK03, AAV-KP1, AAV-KP2, and AAV-KP3 capsid proteins and variants thereof.

For eukaryotic cells, expression control sequences typically include a promoter, an enhancer, such as one derived from an immunoglobulin gene, SV40, cytomegalovirus, etc. (see above), and a polyadenylation sequence which may include splice donor and acceptor sites. The polyadenylation sequence generally is inserted following the transgene sequences and before the 3′ ITR sequence. In some embodiments, the polyadenylation sequence comprises a SV40 polyA or bovine GH polyA sequence. In some embodiments, the AAV vector comprises a 5′ UTR between the promoter and the coding sequence. In some embodiments, the 5′ UTR sequence comprises an intron. In some embodiments, the intron is artificial, derived from an insulin 5′ UTR, or derived from the hemoglobulin subunit beta (HBB) locus. Selection of these and other common vector and regulatory elements are conventional, and many such sequences are available. One of skill in the art may select among these expression control sequences without departing from the scope of this disclosure. Suitable promoter/enhancer sequences may be selected by one of skill in the art using the guidance provided by this application. Such selection is a routine matter and is not a limitation of the molecule or construct.

Exemplary publications relating to AAV vectors and virions include, but are not limited to, U.S. Publication Nos. 2020/0024616, 2015/0176027, 2015/0023924, 2014/0348794, 2014/0242031, and 2012/0164106; all of which are incorporated by reference herein in their entireties.

### Delivery Routes

The polynucleotides and vectors described herein may be delivered via various routes. Non-limiting examples include intraparenchymal delivery, intra-CSF delivery, intramuscular delivery, and systemic delivery (e.g., intravenous or intra-arterial).

In some embodiments, a polynucleotide of the present disclosure may be delivered to the intestine or pancreas of a subject via a minimally invasive endoscopic procedure (e.g., using a catheter). Various endoscopic procedures and devices are known and contemplated herein.

In some embodiments, a depositing device comprising a depositing element is used to deliver a polynucleotide of the present disclosure to a deposit site, such as a pancreatic deposit site.

A depositing device, in some embodiments, comprises a device for implanting, placing, seeding, inserting, spraying, topically applying, and/or otherwise depositing a polynucleotide or a pharmaceutical composition comprising a polynucleotide of the present disclosure at a “deposit site” of a patient. The depositing device comprises one or more needle(s) positioned on a distal portion of the depositing device. In some embodiments, the distal end of the depositing device is delivered into the patient through the mouth, and advanced through a wall of the gastrointestinal tract to a location proximate the pancreas. The depositing device, for example, can be delivered through a working channel of a gastrointestinal endoscope that has been delivered through the mouth of the patient. The depositing device can be delivered alongside a gastrointestinal endoscope that has been delivered through the mouth of the patient, for example. In some embodiments, the pancreatic deposit site comprise one or more sites selected from the group consisting of: intra-parenchymal space; anterior pararenal space; intraductal space; intra-arterial space of an artery that feeds at least a portion of the pancreas; and combinations thereof. Exemplary depositing devices and systems are described, for example, in WO 2022/174091 (e.g., the REJUVA® System) and WO 2016/011269, the entire contents of each of which are incorporated herein by reference.

In some embodiments, the delivering of the polynucleotide of the present disclosure comprises at least a first delivery in which a minimum volume of a pharmaceutical composition comprising the polynucleotide is delivered into the pancreatic parenchyma, and the minimum volume of the composition comprises a volume sufficient to cause at least a portion of the volume of the composition to exit into the anterior pararenal space, spread, and re-enter the pancreas. The method can further comprise at least a second delivery of the composition comprising the polynucleotide to one or more additional deposit sites proximate the tail of the pancreas.

In some embodiments, the delivering of the pharmaceutical composition comprises at least a first delivery in which a minimum volume of pharmaceutical composition is delivered into the pancreatic parenchyma, and the minimum volume of the pharmaceutical composition comprises a volume of at least 2 ml, at least 3 ml, and/or at least 5 ml.

In some embodiments, the depositing device is advanced to the selected one or more pancreatic deposit sites under image guidance. The image guidance can comprise: endoscopic ultrasound guidance; CT guidance; and/or MRI guidance.

In some embodiments, the therapeutic benefit is achieved for a time period of at least 6 months.

In some embodiments, the selected one or more pancreatic deposit sites comprise locations within 10 cm, 7.5 cm, 5 cm, and/or 3 cm of a portion of the pancreas, and the portion of the pancreas comprises the tail, the neck, the body, the head, and/or the uncinate process.

In some embodiments, the pharmaceutical composition and/or the at least one depositing element is configured to be visualized by an imaging device, and the method further comprises visualizing the pharmaceutical composition and/or the at least one depositing element with the imaging device to confirm proper delivery of the pharmaceutical composition.

In some embodiments, the method further comprises delivering an imaging agent through the at least one depositing element and visualizing the delivery of the imaging agent with an imaging device to confirm subsequently proper delivery of the pharmaceutical composition.

In some embodiments, the method further comprises pre-loading the depositing device with the pharmaceutical composition. The pharmaceutical composition can be loaded into the depositing device from the distal end of the depositing device.

In some embodiments, the delivering of the pharmaceutical composition is performed at a pressure of at least 3 mmHg. In some embodiments, the delivering of the pharmaceutical composition is performed at a pressure of no more than 25 mmHg.

In some embodiments, the delivering of the pharmaceutical composition is performed at a flow rate of at least 1 ml/min. In some embodiments, the delivering of the pharmaceutical composition is performed at a flow rate of no more than 5 ml/min.

In some embodiments, the at least one depositing element comprises multiple fenestrations along its length.

In some embodiments, the method further comprises confirming the at least one depositing element is in a proper location prior to delivering the pharmaceutical composition.

In some embodiments, the method further comprises the delivery of a permeability-enhancing agent prior to the delivery of the pharmaceutical composition and/or simultaneously with the delivery of the pharmaceutical composition. The delivery of the permeability-enhancing agent can be performed locally and/or intravenously. The permeability-enhancing agent can comprise an agent selected from the group consisting of: hyaluronidase; collagenase; losartan; and combinations thereof. The pharmaceutical composition can comprise a coformulation of the pharmaceutical composition and the permeability-enhancing agent.

In some embodiments, the method further comprises heating tissue proximate the selected one or more pancreatic deposit sites to a temperature above 39° C. prior to, during, and/or after the delivery of the pharmaceutical composition.

In some embodiments, the method further comprises delivering a dissemination-blocking material that is configured to prevent undesired dissemination of the pharmaceutical composition to non-target locations. The dissemination-blocking material can comprise a viscous substance and/or a polymer.

In some embodiments, the method further comprises positioning a blocking element in the patient, and the blocking element is configured to prevent undesired dissemination of the pharmaceutical composition to non-target locations.

In some embodiments, the method further comprises removing at least a portion of the pharmaceutical composition from a deposit site location after the delivery of the pharmaceutical composition begins.

Additional aspects and embodiments of the delivery device and methods are described in International Publication Number WO 2022/174091 (International Application Number PCT/US2022/016200), incorporated herein by reference in its entirety.

### Host Cells

The disclosure, in some embodiments, provides a host cell comprising the polynucleotides, vectors, vector genomes, recombinant AAV vectors, or fusion proteins described herein. In some embodiments, the host cell comprises a pancreatic islet cell, such as a beta cell.

Where the present gene delivery system is constructed on the basis of viral vector construction, delivery can be performed using conventional infection methods known in the art. Physical methods to enhance delivery both viral and non-viral polynucleotides or polypeptides include electroporation, gene bombardment, sonoporation, magnetofection, hydrodynamic delivery and the like, all of which are known to those of skill in the art. Eukaryotic and prokaryotic host cells, including mammalian cells as hosts for expression of the polypeptide disclosed herein are well known in the art and include many immortalized cell lines available from the American Type Culture Collection (ATCC), such as Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are bacterial cells and fungal cells. Fungal cells include yeast and filamentous fungus cells.

### Therapeutic Uses

The gene therapy described herein may be used to treat metabolic disorders, such as obesity-related metabolic disorders. Exemplary obesity-related metabolic disorders include, but are not limited to, prediabetes, Type 2 diabetes (T2D), cardiovascular disease, polycystic ovary syndrome (PCOS), non-alcoholic fatty liver disease (NAFLD), and non-alcoholic steatohepatitis (NASH).

In some embodiments, the polynucleotides or polypeptides (fusion proteins) described herein (e.g., encoding GLP-1) are used to treat T2D. Treatment of T2D can include, in some embodiments, reduction in HbA1c, reduction in fasting glucose, improved time in a normal range of blood glucose levels, and/or improvement in hyperglycemia. It may also include reduction in or elimination of the need for exogenous insulin, reduction in or elimination of need for exogenous GLP-1 receptor agonists, without causing increased rates of nausea, diarrhea, vomiting, constipation, or abdominal pain.

Reductions in blood glucose, in some embodiments, also leads to additional beneficial outcomes including reductions in the rates or progression of retinopathy, nephropathy, neuropathy, myocardial infarction, microvascular disease related to diabetes, and prevention or reduced incidence of end stage kidney disease.

Additional possible benefits, in some embodiments, include a reduced rate of cognitive decline and/or reduction in major adverse cardiovascular (CV) events (MACE), e.g., reduction in composite of CV death, nonfatal myocardial infarction (MI), and/or nonfatal stroke.

Other forms of diabetes where glucoregulatory hormone production in the pancreatic beta cells may in principle be helpful include “double diabetes” (when T1D patients also get T2D); gestational diabetes; and pre-diabetes.

Other consequences of T2D that may improve include, for example, hypertension, hypertriglyceridemia, hypercholesterolemia heart disease, diabetic heart disease, heart failure, diabetic heart failure, and/or diastolic dysfunction.

Other diseases that co-occur with T2D and obesity and are thought to be related (but not treated with insulin unless T2D indicated) include, for example, polycystic ovarian syndrome (PCOS), hyperandrogenism, fertility issues, menstrual dysfunction, hirsutism, dementia, Alzheimer's disease, cognitive decline, cancer such as liver cancer, ovarian cancer, breast cancer, endometrial cancer, cholangiocarcinoma, adenocarcinoma, glandular tissue tumor(s), stomach cancer, large bowel cancer, and/or prostate cancer, psoriasis, hypogonadism, insufficient total testosterone levels, and/or insufficient free testosterone level.

In some embodiments, the polynucleotides or polypeptides (fusion proteins) described herein (e.g., encoding PYY) are used to treat NAFLD.

In some embodiments, the polynucleotides or polypeptides (fusion proteins) described herein (e.g., encoding PYY) are used to treat NASH.

In some embodiments, the polynucleotides or polypeptides (fusion proteins) described herein (e.g., encoding PYY) are used to treat obesity. Other diseases that co-occur with obesity, and thus would be expected to improve with weight loss include, for example, gastroesophageal reflux disease (GERD), sleep apnea, arthritis, hypertension coronary artery disease (e.g., as a secondary prevention), stroke, transient ischemic attack (TIA), diastolic dysfunction, myocardial infarction, and heart failure.

As used herein, “treating” refers to the administration of one or more therapeutics (e.g., polynucleotides, vectors, or fusion proteins) with the expectation that the subject may have a resulting benefit due to the administration. A subject may be any mammal, including non-human primate and human subjects. Typically, a subject is a human subject. In some embodiments, the amount of polynucleotide, vector, or fusion protein administered is an effective amount. An “effective amount” or a “therapeutically effective amount” of a composition (e.g., pharmaceutical composition comprising the polynucleotides, vectors, and/or fusion proteins provided herein) is based, at least in part, on the target tissue, target cell type, means of administration, physical characteristics of the pharmaceutical composition, other pharmaceutical composition, and other determinants, such as age, body weight, height, sex and general health of the subject. Typically, an effective amount of a pharmaceutical composition provides an improved glucose (blood sugar) level in the subject.

In some embodiments, an effective amount restores glycemic durability in the subject. As used herein, “glycemic durability” refers to a period of time during which a subject's glycemic levels are within physiological ranges (e.g., “ideal glycemic control” or “optimal glycemic control”). According to the American Diabetes Association, the recommended Hb1Ac cut-point for diagnosing diabetes is 6.5%, and individuals are at a high risk (Gillett et al., Diabetes Care. 2009; 32:1327-34). In some embodiments, the glycemic physiological range is glycosylated hemoglobin (HbA1c) value of less than 10%, less than 9%, less than 8%, less than 7.5%, less than 7%, less than 6.5%, less than 6%, or less than 5%. In some embodiments, the effective amount results in the maintenance of the HbA1c value at less than 7%. In some embodiments, the optimal glycemic control is maintained for at least 1 month, at least 2 months, at least 3 months, at least 4 months, at least 5 months, at least 6 months, at least 8 months, at least 10 months, at least 1 year, at least 1.5 years, at least 2 years, at least 2.5 years, at least 3 years, at least 3.5 years, at least 4 years, at least 4.5 years, at least 5 years, or longer. In some embodiments, the optimal glycemic control is maintained without substitution and/or addition of other glucose-lowering agents.

In some embodiments, an effective amount reduces fasting blood glucose relative to baseline. In some embodiments, an effective amount reduces fasting blood glucose by at least 10%, 15%, 20%, 25%, 30%, 35%, 40%, 45%, 50%, 55%, 60%, 65%, 70%, 75%, 80%, 85%, 90%, or more relative to baseline. In some embodiments, the effective amount reduces fasting blood glucose by at least 50% relative to baseline. In some embodiments, the effective amount reduces fasting blood glucose by at least 55% relative to baseline. In some embodiments, the effective amount reduces fasting blood glucose by at least 60% relative to baseline. According to the WHO, normal values for fasting glucose are between 70 mg/dL and 100 mg/dL, while 100 mg/dL to 125 mg/dL represents a pre-diabetic state, and fasting blood glucose above 126 mg/dl indicates the subject is diabetic (WHO, “Mean fasting blood glucose,” who.int/data/gho/indicator-metadata-registry/imr-details/2380). In some embodiments, the effective amount reduces fasting blood glucose in a subject to less than 130 mg/dL, less than 126 mg/dL, less than 120 mg/dL, less than 115 mg/dL, less than 110 mg/dL, less than 105 mg/dL, or less than 100 mg/dL. As used herein, “fasting blood glucose” refers to the blood glucose value (glucose concentration in venous plasma) determined when the subject has fasted (without any food except water) for at least 6, 7, 8, 9, 10, 11, 12, 14, 16, 18, 20, 22, 24, or more hours (see, e.g., WHO, “Mean fasting blood glucose”). “Baseline” refers to a subject's levels (e.g., blood glucose levels) before beginning treatment.

In some embodiments, an effective amount increases fasting insulin relative to baseline. In some embodiments, an effective amount increases fasting insulin by at least 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 4-fold, 5-fold, or more relative to baseline. In some embodiments, the effective amount increases fasting insulin by 2-fold relative to baseline. In some embodiments, the effective amount increases fasting insulin by, or by at least, 2.8-fold relative to baseline. In some embodiments, the effective amount increases fasting insulin by 3-fold relative to baseline. As used herein, “fasting insulin” refers to the insulin level determined when the subject has fasted (without any food except water) for at least 6, 7, 8, 9, 10, 11, 12, 14, 16, 18, 20, 22, 24, or more hours. “Baseline” refers to a subject's levels (e.g., insulin levels) before beginning treatment.

In some embodiments, an effective amount significantly improves glucose tolerance relative to baseline. Glucose tolerance refers to a subject's ability to control plasma glucose and/or plasma insulin levels when glucose intake varies. It may be measured using any method in the art, including oral glucose tolerance tests (OGTTs), such as a glucose challenge test during which the subject drinks a glass of concentrated glucose solution (e.g., 50 g of glucose dissolved in 250-300 mL of water) and the subject's blood sugar level is measured in the blood at least 1 hour later. In some embodiments, glucose tolerance is measured by comparing a fasting blood glucose level to the blood glucose level 1-3 hours after consuming the concentrated glucose solution. According to the American Diabetes Association, a blood glucose (sugar) concentration of less than 140 mg/dL is normal, 140 mg/dL-199 mg/dL indicates prediabetes, and 200 mg/dL or more indicates diabetes (diabetes.org/diabetes/alc/diagnosis). In some embodiments, the subject has a blood sugar level of less than 200 mg/dL, less than 190 mg/dL, less than 180 mg/dL, less than 170 mg/dL, less than 160 mg/dL, less than 150 mg/dL, less than 140 mg/dL, less than 130 mg/dL, or less. In some embodiments, the subject's blood sugar level is less than 140 mg/dL after treatment. In some embodiments, the subject's blood sugar level is reduced 5 mg/dL, 6 mg/dL, 7 mg/dL, 8 mg/dL, 9 mg/dL, 10 mg/dL, 15 mg/dL, 20 mg/dL, 25 mg/dL, 30 mg/dL, 35 mg/dL, 40 mg/dL, 45 mg/dL, 50 mg/dL, or more relative to baseline.

In some embodiments, an effective amount significantly improves glucose-stimulated insulin secretion relative to baseline. Glucose-stimulated insulin secretion (GSIS) can be measured using any method known in the art, for example, hyperinsulinemic-euglycemic clamp method, the hyperglycemic clamp method, or extrapolating from surrogate measures of insulin sensitivity (e.g., intravenous glucose tolerance test data, fasting blood samples, and the quantitative insulin sensitivity check index). In some embodiments, the GSIS is increased from baseline following treatment. In some embodiments, the GSIS is increased at least 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 4-fold, 5-fold, or more relative to baseline.

In some embodiments, the body weight of the subject does not change significantly relative to baseline. In some embodiments, the body weight of the subject changes by less than 10%, 9%, 8%, 7%, 6%, 5%, 4%, 3%, 2%, 1% relative to baseline. In some embodiments, the body weight of the subject stays the same relative to baseline.

In some embodiments, the effective amount of a pharmaceutical composition results in a subject having T2D in remission (that is, the subject maintains physiological levels of blood glucose). In some embodiments, the effective amount is a single dose, two doses, three doses, four doses, five doses, six doses, or more doses. In some embodiments, the effective amount is sufficient for long-term restoration of beta cell function and/or reduction of therapeutic burden (e.g., workload of healthcare experienced by the subject and its impact on the subject's well-being). In some embodiments, “long term restoration” means 1 year, 2 years, 3 years, 4 years, 5 years, 6 years, 7 years, 8 years, 9 years, 10 years or longer, including complete and permanent remission.

In some embodiments, additional treatments are administered in addition to the pharmaceutical compositions provided herein. Exemplary additional treatments include treatments for T2D, such as amylinomimetic drugs, alpha-glucosidase inhibitors, biguanides, dopamine agonists, dipeptidyl peptidase-4 (DPP-4) inhibitors, GLP-1 receptor agonists, meglitinides, statins, sodium-glucose transporter (SGLT) 2 inhibitors, sulfonylureas, thiazolidinediones, insulin, and combinations thereof. In some embodiments, additional treatments are not administered to the subject.

In some embodiments, the polynucleotides, vectors, or fusion proteins may be administered as part of a pharmaceutical composition. The term “pharmaceutical composition” refers to the combination of an active agent with a carrier, inert or active, making the composition especially suitable for therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

The pharmaceutical composition may further comprise one or more pharmaceutically-acceptable excipients. In addition to traditional excipients such as any and all solvents, dispersion media, diluents, or other liquid vehicles, dispersion or suspension aids, surface active agents, isotonic agents, thickening or emulsifying agents, preservatives, pharmaceutically-acceptable excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof. In some embodiments, the pharmaceutical compositions comprise at least one additional active substance, such as, for example, a therapeutically-active substance.

The compositions may be sterile, pyrogen-free or both sterile and pyrogen-free. For the purposes of the present disclosure, the phrase “active ingredient” generally refers to the polynucleotides, vectors, or fusion proteins described herein. Formulations of the compositions described herein may be prepared by any method known or hereafter developed in the art of pharmacology. In general, such preparatory methods include the step of bringing the active ingredient (e.g., polynucleotide) into association with an excipient and/or one or more other accessory ingredients, and then, if necessary and/or desirable, dividing, shaping and/or packaging the product into a desired single- or multi-dose unit. Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The polynucleotides, vectors, or fusion proteins described herein may be administered by any route that results in a therapeutically effective outcome. These include, but are not limited, to intradermal, intramuscular, intranasal, and/or subcutaneous administration. In some embodiments, the polynucleotides, vectors, or fusion proteins described herein are delivered locally instead of systemically. In some embodiments, the polynucleotides, vectors, or fusion proteins are delivered to a pancreatic islet cell (e.g., a beta cell). In some embodiments, the polynucleotides, vectors, or fusion proteins are delivered to the pancreatic islet cell via an endoscopic procedure.

The present disclosure also contemplates combination therapies using, for example, the REVITA® System, which is a minimally invasive, outpatient, endoscopic, one-time procedural therapy. The REVITA® System includes a specially designed control console and a novel single-use balloon catheter. The console is used to monitor the procedure, while the physician uses the catheter to apply heat to the duodenum. The REVITA® System may be used, in some embodiments, as an adjunct combination therapy.

### ADDITIONAL EMBODIMENTS

Additional embodiments are described in the following numbered paragraphs:
1. A polynucleotide comprising a glucoregulatory hormone coding sequence and an insulin coding sequence.
2. The polynucleotide of paragraph 1, wherein the glucoregulatory hormone coding sequence and the insulin coding sequence are arranged such that a functional glucoregulatory hormone and a functional insulin are produced in vivo following administration to a subject.
3. The polynucleotide of paragraph 1, wherein the glucoregulatory hormone coding sequence is nested in the insulin coding sequence.
4. The polynucleotide of paragraph 1, wherein the insulin coding sequence comprises, 5′ to 3′, a signal peptide coding sequence, a B-chain coding sequence, a C-peptide coding sequence, and an A-chain coding sequence.
5. The polynucleotide of paragraph 4, wherein the glucoregulatory hormone coding sequence is located between the signal peptide coding sequence and the B-chain coding sequence.
6. The polynucleotide of paragraph 4, wherein the glucoregulatory hormone coding sequence is nested in the B-chain coding sequence.
7. The polynucleotide of paragraph 4, wherein the glucoregulatory hormone coding sequence is located between the B-chain coding sequence and the C-peptide coding sequence.
8. The polynucleotide of paragraph 4, wherein the glucoregulatory hormone coding sequence is nested in the C-peptide coding sequence.
9. The polynucleotide of paragraph 4, wherein the glucoregulatory hormone coding sequence is located between the C-peptide coding sequence and the A-chain coding sequence.
10. The polynucleotide of paragraph 4, wherein the glucoregulatory hormone coding sequence is nested in the A-chain coding sequence.
11. The polynucleotide of paragraph 4, wherein the glucoregulatory hormone coding sequence is located downstream from the A-chain coding sequence.
12. The polynucleotide of any one of paragraphs 1-11, wherein the glucoregulatory hormone coding sequence is flanked by a first PCSK1 (PC1/3) and/or PCSK2 (PC2) enzyme processing sequence, optionally a native PCSK1 or PCSK2 enzyme processing sequence or an artificial PCSK1 or PCSK2 enzyme processing sequence.
13. The polynucleotide of any one of paragraphs 1-11, wherein the glucoregulatory hormone coding sequence is flanked by a first PCSK1 and/or PCSK2 enzyme processing sequence and a second PCSK1 and/or PCSK2 enzyme processing sequence, optionally a native PCSK1 or PCSK2 enzyme processing sequence or an artificial PCSK1 or PCSK2 enzyme processing sequence.
14. The polynucleotide of any one of paragraphs 1-3, comprising, 5′ to 3′, a signal peptide coding sequence, a glucoregulatory hormone coding sequence, a B-chain coding sequence, a first PCSK1 or PCSK2 enzyme processing sequence, a C-peptide coding sequence, a second PCSK1 or PCSK2 enzyme processing sequence, and an A-chain coding sequence.
15. The polynucleotide of any one of paragraphs 1-3, comprising, 5′ to 3′, a signal peptide coding sequence, a B-chain coding sequence, a glucoregulatory hormone coding sequence, a first PCSK1 or PCSK2 enzyme processing sequence, a C-peptide coding sequence, a second PCSK1 or PCSK2 enzyme processing sequence, and an A-chain coding sequence.
16. The polynucleotide of any one of paragraphs 1-3, comprising, 5′ to 3′, a signal peptide coding sequence, a B-chain coding sequence, a first PCSK1 or PCSK2 enzyme processing sequence, a glucoregulatory hormone coding sequence, a C-peptide coding sequence, a second PCSK1 or PCSK2 enzyme processing sequence, and an A-chain coding sequence.
17. The polynucleotide of any one of paragraphs 1-3, comprising, 5′ to 3′, a signal peptide coding sequence, a B-chain coding sequence, a first PCSK1 or PCSK2 enzyme processing sequence, a glucoregulatory hormone coding sequence nested in a C-peptide coding sequence, a second PCSK1 or PCSK2 enzyme processing sequence, and an A-chain coding sequence.
18. The polynucleotide of any one of paragraphs 1-3, comprising, 5′ to 3′, a signal peptide coding sequence, a B-chain coding sequence, a first PCSK1 or PCSK2 enzyme processing sequence, a C-peptide coding sequence, a glucoregulatory hormone coding sequence, a second PCSK1 or PCSK2 enzyme processing sequence, and an A-chain coding sequence.
19. The polynucleotide of any one of paragraphs 1-3, comprising, 5′ to 3′, a signal peptide coding sequence, a B-chain coding sequence, a first PCSK1 or PCSK2 enzyme processing sequence, a C-peptide coding sequence, a second PCSK1 or PCSK2 enzyme processing sequence, a glucoregulatory hormone coding sequence, and an A-chain coding sequence.
20. The polynucleotide of any one of paragraphs 1-3, comprising, 5′ to 3′, a signal peptide coding sequence, a B-chain coding sequence, a first PCSK1 or PCSK2 enzyme processing sequence, a C-peptide coding sequence, a second PCSK1 or PCSK2 enzyme processing sequence, an A-chain coding sequence, and a glucoregulatory hormone coding sequence.
21. The polynucleotide of any one of the preceding paragraphs, wherein the glucoregulatory hormone is selected from glucagon, GLP-1, oxyntomodulin, glicentin, glicentin-related polypeptide (GRPP), major proglucagon fragment, intervening peptide 1 (IP-1), intervening peptide 2 (IP-2), GLP-2, glucose-dependent insulinotropic peptide (GIP), peptide tyrosine (PYY), Cholecystokinin (CCK), somatostatin, oxyntomodulin, Ghrelin, amylin, glucagon, leptin, follistatin, insulin-like growth factor 1 (IGF1), vasoactive intestinal peptide (VIP), and growth hormone 1 (GH1), and peptides, variants and fusions thereof, optionally any one of SEQ ID NOs: 30-58.
22. The polynucleotide of paragraph 21, wherein the glucoregulatory hormone is a wild-type human GLP-1.
23. The polynucleotide of paragraph 21, wherein the GLP-1 is a variant human GLP-1 comprising a Gly8 substitution, relative to a wild-type human GLP-1.
24. The polynucleotide of any one of the preceding paragraphs comprising a nucleotide sequence having at least 80%, at least 85%, at least 90%, at least 95%, or 100% identity to the nucleotide sequence of any one of SEQ ID NOs: 59-71.
25. The polynucleotide of any one of the preceding paragraphs comprising a nucleotide sequence encoding a polypeptide comprising an amino acid sequence having at least 80%, at least 85%, at least 90%, at least 95%, or 100% identity to the amino acid sequence of any one of SEQ ID NOs: 72-84.
26. The polynucleotide of any one of the preceding paragraphs, wherein the polynucleotide is operably linked to a promoter sequence, preferably a pancreatic islet cell promoter sequence, more preferably a pancreatic islet beta cell promoter sequence, optionally selected from: the human Insulin promoter, the mouse Insulin2 promoter, the mouse Insulin1 promoter, the rat Insulin2 promoter, the rat Insulin1 promoter, Slc2a, IAPP, NKX6.1, DLK1, MafA, Slc30a8/Znt8, PCSK1, and ADCYAP1.
27. The polynucleotide of any one of the preceding paragraphs, further comprising an enhancer sequence.
28. A vector comprising the polynucleotide of any one of the preceding paragraphs.
29. The vector of paragraph 28, wherein the vector is a nonviral vector, optionally a plasmid, bacterial artificial chromosome, yeast artificial chromosome, or minicircle.
30. The vector of paragraph 28, wherein the vector is a viral vector, optionally selected from selected from a retroviral vector, an adenovirus vector, a Herpes simplex virus (HSV) vector, and an adeno-associated virus (AAV) vector.
31. A recombinant adeno-associated virus (AAV) vector genome comprising the polynucleotide of any one of paragraphs 1-27.
32. The recombinant AAV vector genome of paragraph 31 further comprising inverted terminal repeat (ITR) sequences, optionally flanking the polynucleotide.
33. The recombinant AAV vector genome of paragraph 32, wherein the ITR sequences are selected from AAV1, AAV2, AAV3, AAV4, AAV5, AAV6, AAV7, AAV8, AAV9, AAV10 (AAVrh10), and AAV11 ITR sequences.
34. A recombinant adeno-associated virus (AAV) vector comprising (a) the recombinant AAV vector genome of any one of paragraphs 31-33 and (b) a capsid protein.
35. The recombinant AAV vector of paragraph 34, wherein the capsid protein is selected from AAV1, AAV2, AAV3, AAV4, AAV5, AAV6, AAV7, AAV8, AAV9, AAV10, AAV11, Rh10, Rh74, AAV-218, AAV-DJ, AAV-LK03, AAV-KP1, AAV-KP2, and AAV-KP3 capsid proteins, and variants thereof.
36. A fusion protein encoded by the polynucleotide of any one of paragraphs 1-27.
37. A fusion protein comprising an amino acid sequence having at least 80%, at least 85%, at least 90%, at least 95%, or 100% identity to the amino acid sequence of any one of SEQ ID NOS: 72-84.
38. A host cell comprising the polynucleotide of any one of paragraphs 1-27, the vector of any one of paragraphs 28-30, the AAV vector genome of any one of paragraphs 31-33, the recombinant AAV vector of paragraph 34 or 35, or the fusion protein of paragraph 36 or 37.
39. A method comprising delivering to a pancreatic islet cell the polynucleotide of any one of paragraphs 1-27, the vector of any one of paragraphs 28-30, the AAV vector genome of any one of paragraphs 31-33, the recombinant AAV vector of paragraph 34 or 35, or the fusion protein of paragraph 36 or 37.
40. A method comprising administering to a subject the polynucleotide of any one of paragraphs 1-27, the vector of any one of paragraphs 28-30, the AAV vector genome of any one of paragraphs 31-33, the recombinant AAV vector of paragraph 34 or 35, or the fusion protein of paragraph 36 or 37.
41. The method of paragraph 40, wherein the subject has an obesity-related metabolic disorder selected from the group consisting of: prediabetes, Type 2 diabetes, cardiovascular disease, polycystic ovary syndrome (PCOS), non-alcoholic fatty liver disease (NAFLD), and non-alcoholic steatohepatitis (NASH).
42. A method of treating an obesity-related metabolic disorder, the method comprising administering to a subject in need thereof an effective amount of the polynucleotide of any one of paragraphs 1-27, the vector of any one of paragraphs 28-30, the AAV vector genome of any one of paragraphs 31-33, the recombinant AAV vector of paragraph 34 or 35, or the fusion protein of paragraph 36 or 37.
43. The method of paragraph 40, wherein the obesity-related metabolic disorder is selected from the group consisting of: prediabetes, Type 2 diabetes, cardiovascular disease, polycystic ovary syndrome (PCOS), non-alcoholic fatty liver disease (NAFLD), and non-alcoholic steatohepatitis (NASH).
44. The method of any one of paragraphs 39-43, wherein the administering is via endoscopic delivery to the pancreas or a region near the pancreas.
45. The method of paragraph 42, wherein the effective amount restores glycemic durability in the subject.
46. The method of paragraph 42, wherein the effective amount reduces fasting blood glucose by at least 50% relative to baseline.
47. The method of paragraph 42, wherein the effective amount increases fasting insulin by at least 2-fold relative to baseline.
48. The method of paragraph 42, wherein the effective significantly improves glucose tolerance relative to baseline.
49. The method of paragraph 42, wherein the effective significantly improves glucose-stimulated insulin secretion relative to baseline.
50. The method of paragraph 48 or 49, wherein body weight of the subject does not change significantly relative to baseline.
51. The method of paragraph 42, wherein the effective amount is a single dose, and the single dose is sufficient for long-term restoration of beta cell function and/or reduction in therapeutic burden.
52. A method of treating an obesity-related metabolic disorder, the method comprising administering to a subject in need thereof an effective amount of an adeno-associated virus (AAV) vector genome, wherein the AAV vector genome comprises a polynucleotide comprising a human pancreatic islet beta cell-specific promoter operably linked to a human GLP-1 receptor agonist coding sequence, optionally a human GLP-1 coding sequence, and wherein
   (a) the effective amount restores glycemic durability in the subject;
   (b) the effective amount reduces fasting blood glucose by at least 50% relative to baseline;
   (c) the effective amount increases fasting insulin by at least 50% by at least 2-fold relative to baseline;
   (d) the effective significantly improves glucose tolerance relative to baseline;
   (e) the effective significantly improves glucose-stimulated insulin secretion relative to baseline;
   (f) the body weight of the subject does not change significantly relative to baseline; and/or
   (g) the effective amount is a single dose, and the single dose is sufficient for long-term restoration of beta cell function and/or reduction in therapeutic burden.
53. The method of paragraph 52, wherein the method comprises administering to a subject in need thereof an effective amount of an AAV vector comprising the AAV vector genome and a capsid protein.
54. The method of paragraph 53, wherein the capsid protein is selected from AAV1, AAV2, AAV3, AAV4, AAV5, AAV6, AAV7, AAV8, AAV9, AAV10, AAV11, Rh10, Rh74, AAV-218, AAV-DJ, AAV-LK03, AAV-KP1, AAV-KP2, and AAV-KP3 capsid proteins, and variants thereof.
55. An endoscopic gene therapy method, comprising:
   advancing a depositing device comprising at least one depositing element to at least one pancreatic deposit site in a patient having a metabolic disease and/or a pancreatic disease; and
   delivering an effective amount of a treatment agent through the at least one depositing element into the at least one pancreatic deposit site,
   wherein the treatment agent comprises an adeno-associated virus (AAV) vector that comprises an AAV vector genome, wherein the AAV vector genome comprises a polynucleotide comprising a human pancreatic islet beta cell-specific promoter operably linked to (a) a human GLP-1 receptor agonist coding sequence, optionally a human GLP-1 coding sequence, or (b) a peptide tyrosine tyrosine (PYY) coding sequence.
56. The method of paragraph 55, wherein the at least one depositing element comprises at least one needle positioned on a distal portion of the depositing device.
57. The method of paragraph 55 or 56, wherein the distal end of the depositing device is delivered into the patient through the mouth, and advanced through a wall of the gastrointestinal tract to a location proximate the pancreas, optionally wherein (a) the depositing device is delivered through a working channel of a gastrointestinal endoscope that has been delivered through the mouth of the patient of (b) the depositing device is delivered alongside a gastrointestinal endoscope that has been delivered through the mouth of the patient.
58. The method of any one of paragraphs 55-57, wherein:
   the metabolic disease is selected from the group consisting of: Type 1 diabetes; Type 2 diabetes; nonalcoholic fatty liver disease (NAFLD); nonalcoholic steatohepatitis (NASH);
   obesity; and combinations thereof; or the pancreatic disease is selected from the group consisting of: pancreatitis; pancreatic cancer; hyperinsulinism; and combinations thereof.
59. The method of any one of paragraphs 55-58, wherein the at least one pancreatic deposit site is selected from the group consisting of: intraparenchymal space; anterior pararenal space; intraductal space; intraarterial space of an artery that feeds at least a portion of the pancreas; and combinations thereof, preferably wherein the at least one pancreatic deposit site is the intraparenchymal space.
60. The method of any one of paragraphs 55-59, wherein the delivering of a treatment agent comprises at least a first delivery in which a minimum volume of the treatment agent is delivered into the pancreatic parenchyma, and wherein the minimum volume of treatment agent comprises a volume sufficient to cause at least a portion of the volume of the treatment agent to exit into the anterior pararenal space, spread, and re-enter the pancreas, optionally wherein the method further comprises at least a second delivery of the treatment agent to at least one additional deposit site proximate the tail of the pancreas.
61. The method of any one of paragraphs 55-60, wherein the delivering of a treatment agent comprises at least a first delivery in which a minimum volume of treatment agent is delivered into the pancreatic parenchyma, and wherein the minimum volume of the treatment agent comprises a volume of at least 2 ml, at least 3 ml, and/or at least 5 ml.
62. The method of any one of paragraphs 55-61, wherein the depositing device is advanced to the at least one pancreatic deposit site under image guidance, optionally, wherein the image guidance comprises: endoscopic ultrasound guidance; computerized tomography (CT) guidance; and/or magnetic Resonance Imaging (MRI) guidance.
63. The method of any one of paragraphs 55-62, wherein the at least one pancreatic deposit site comprises locations within 10 cm, 7.5 cm, 5 cm, and/or 3 cm of a portion of the pancreas, and wherein the portion of the pancreas comprises the tail, the neck, the body, the head, and/or the uncinate process.
64. The method of any one of paragraphs 55-63, wherein the treatment agent and/or the at least one depositing element is configured to be visualized by an imaging device, and wherein the method further comprises visualizing the treatment agent and/or the at least one depositing element with the imaging device to confirm proper delivery of the treatment agent.
65. The method of any one of paragraphs 55-64, further comprising delivering an imaging agent through the at least one depositing element and visualizing the delivery of the imaging agent with an imaging device to confirm subsequently proper delivery of the treatment agent.
66. The method of any one of paragraphs 55-65, further comprising pre-loading the depositing device with the treatment agent, optionally wherein the treatment agent is loaded into the depositing device from the distal end of the depositing device.
67. The method of any one of paragraphs 55-66, wherein the delivering of a treatment agent is performed at a pressure of at least 3 mmHg and/or no more than 25 mmHg.
68. The method of any one of paragraphs 55-67, wherein the delivering of a treatment agent is performed at a flow rate of at least 1 ml/min and/or no more than 5 ml/min.
69. The method of any one of paragraphs 55-68, wherein the at least one depositing element comprises multiple fenestrations along its length.
70. The method of any one of paragraphs 55-69, further comprising confirming the at least one depositing element is in a proper location prior to delivering the treatment agent.
71. The method of any one of paragraphs 55-70, further comprising the delivery of a permeability-enhancing agent prior to the delivery of the treatment agent and/or simultaneously with the delivery of the treatment agent, optionally wherein the delivery of the permeability-enhancing agent is performed locally and/or intravenously, optionally wherein the permeability-enhancing agent comprises an agent selected from the group consisting of: hyaluronidase; collagenase; losartan; and combinations thereof, and optionally wherein the treatment agent comprises a coformulation of the treatment agent and the permeability-enhancing agent.
72. The method of any one of paragraphs 55-71, further comprising heating tissue proximate the at least one pancreatic deposit site to a temperature above 39° C. prior to, during, and/or after the delivery of the treatment agent.
73. The method of any one of paragraphs 55-72, further comprising delivering a dissemination-blocking material that is configured to prevent undesired dissemination of the treatment agent to non-target locations, optionally wherein the dissemination-blocking material comprises a viscous substance and/or a polymer.
74. The method of any one of paragraphs 55-73, further comprising positioning a blocking element in the patient, wherein the blocking element is configured to prevent undesired dissemination of the treatment agent to non-target locations.
75. The method of any one of paragraphs 55-74, further comprising removing at least a portion of the treatment agent from a deposit site location after the delivery of the treatment agent begins.
76. The method of any one of paragraphs 55-75, further comprising removing all of the treatment agent.

### EXAMPLES

### Example 1—In Vitro Experiments in Mouse Insulinoma (MIN-6) Cells

DNA expression plasmids were generated to enable transient transfection of different transgenes into a murine pancreatic beta cell model in order to test for each transgene's ability to encode functional GLP-1 peptide production and secretion. The transgenes tested are provided in Table 2 and Table 3 and schematically depicted in FIG. 4. Briefly, INS-GLP-1 hybrids (#01-#13) were cloned into an expression plasmid under the control of a human cytomegalovirus (CMV) promoter. The coding sequence of the human preproglucagon (GCG) gene, which produces GLP-1 as well as additional peptides, including GLP-2 and peptides that contain the glucagon sequence, served as a positive control. Other controls included: no transfection (cells only), empty plasmids, and plasmids expressing a green fluorescence protein (GFP) transgene.

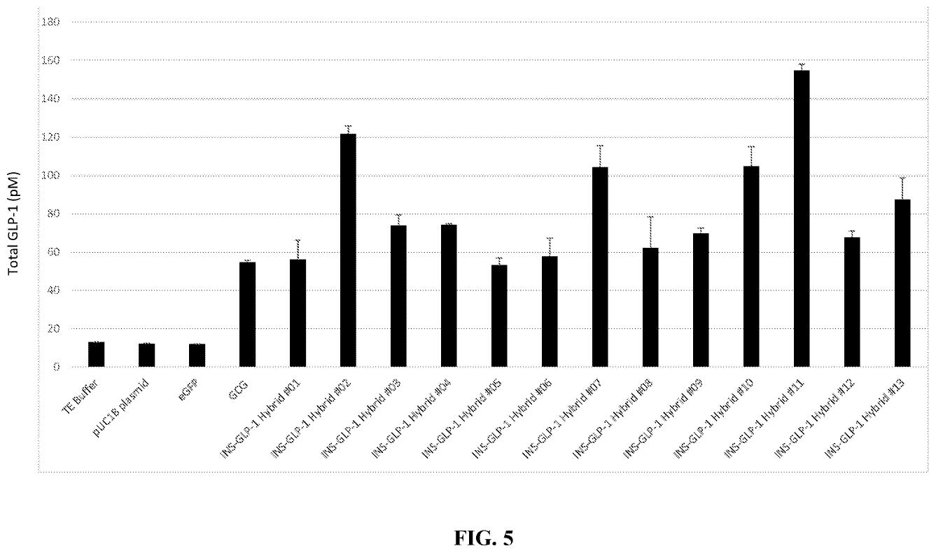

The mouse MIN-6 cell line, a commonly used model of beta cells, was transfected with the expression plasmids described above. Forty-eight hours after transfection, cells were exposed to 25 mM glucose plus 0.100 mM 3-isobutyl-1-methylxanthine (IBMX)) and then the levels of GLP-1 in the supernatants were measured. The results are shown in FIG. 5 and demonstrate that transfection with multiple INS-GLP-1 hybrid expression plasmids resulted in secreted GLP-1.

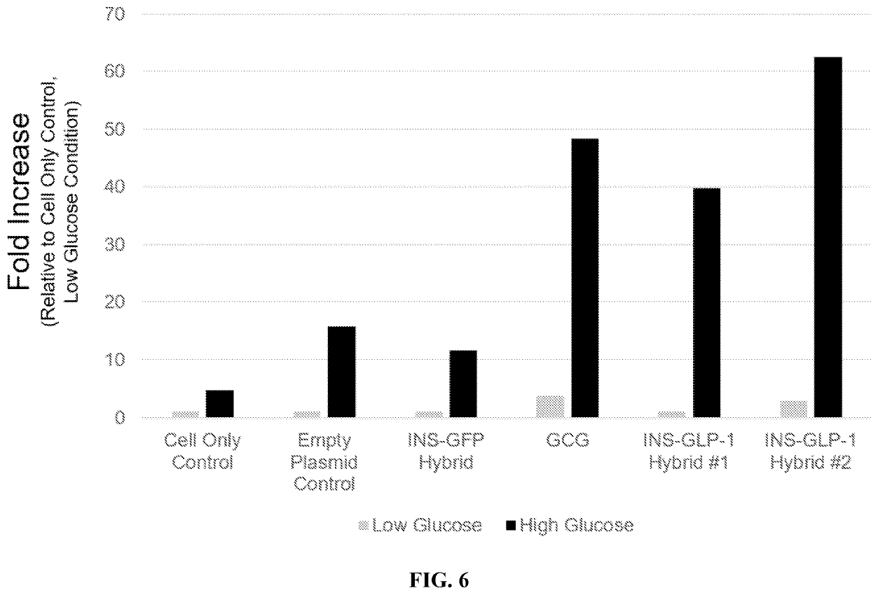

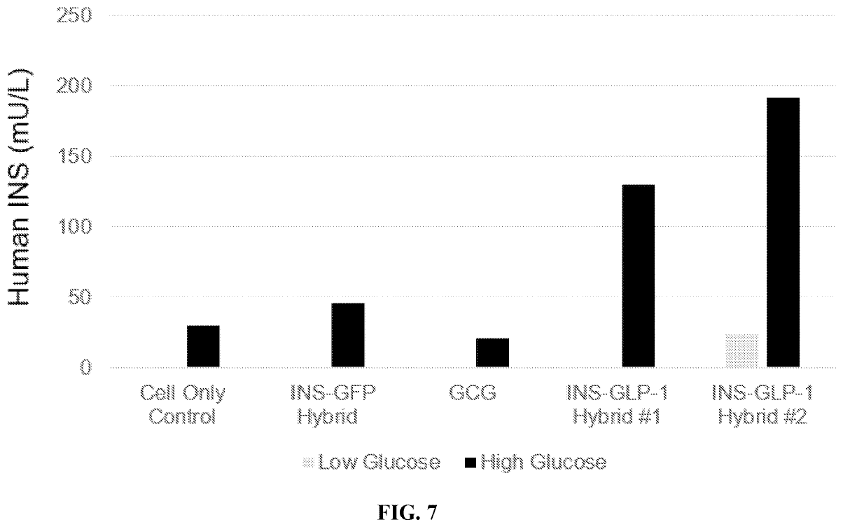

In a further experiment, following a 48-hour incubation with the expression vectors described above, the cells were exposed to either low glucose (2 mM) or high glucose (25 mM glucose+0.100 mM IBMX) for 5 hours to investigate nutrient-responsive transgene secretion into the supernatant of transfected cells. The results are shown in FIG. 6 and FIG. 7 and indicate that the two hybrid transgene constructs produced GLP-1 (FIG. 6) and human insulin (FIG. 7), to be selectively secreted in beta cells in response to high glucose conditions

TABLE 3
Description of Exemplary Fusion Proteins Tested

| | | Amino Acid | |
|---|---|---|---|
| Name | Insulin Insertion Site | Insertion Site | GLP-1 Sequence |
| INS-GLP-1 Hybrid #01 | C-Peptide Insertion, C-terminal | INS Q87-K88 | GCG(K82-P133) |
| INS-GLP-1 Hybrid #02 | C-Peptide Insertion, C-terminal | INS Q87-K88 | GCG(K82-R145) |
| INS-GLP-1 Hybrid #03 | C-Peptide Insertion, N-terminal | INS T54-R55 | GCG(K82-R145) |
| INS-GLP-1 Hybrid #04 | C-Peptide Insertion, Middle | INS G71-P72 | GCG(K82-R145) |
| INS-GLP-1 Hybrid #05 | C-Peptide Insertion, C-terminal | INS Q87-K88 | GCG(K90-R145) |
| INS-GLP-1 Hybrid #06 | C-Peptide Insertion, C-terminal | INS Q87-K88 | GCG(K82-R130) |
| INS-GLP-1 Hybrid #07 | C-Peptide Insertion, N-terminal | INS T54-R55 | GCG(K90-R130) |
| INS-GLP-1 Hybrid #08 | C-Peptide Insertion, Middle | INS G71-P72 | GCG(K90-R130) |
| INS-GLP-1 Hybrid #09 | C-Peptide Insertion, C-terminal | INS Q87-K88 | GCG(K90-R130) |
| INS-GLP-1 Hybrid #10 | C-terminal Fusion | INS after N110 | GCG(K82-R145) |
| INS-GLP-1 Hybrid #11 | C-terminal Fusion | INS after N110 | GCG(K90-R130) |
| INS-GLP-1 Hybrid #12 | In between SP and B-chain | INS A24-F25 | GCG(K82-R145) |
| INS-GLP-1 Hybrid #13 | In between SP and B-chain | INS A24-F25 | GCG(K90-R130) |

GCG = human preproglucagon coding sequence

### Example 2—In Vitro Experiments in Human EndoC-BH5 Cell Model

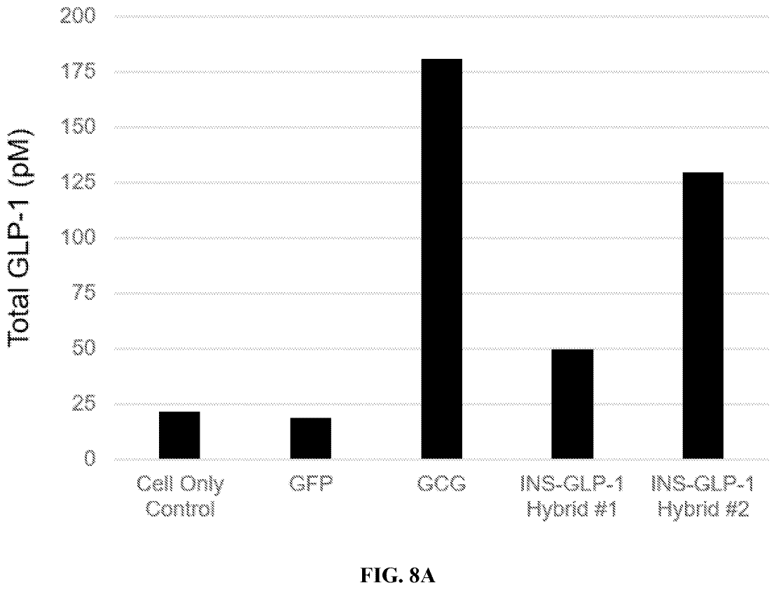

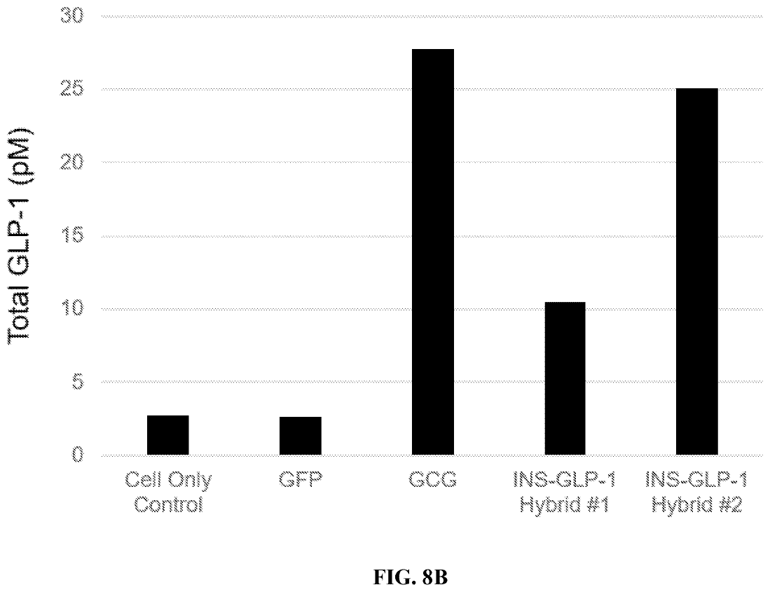

The expression vectors comprising the transgenes described in Example 1 were produced and used to transfect human pancreatic beta cells (EndoC-BH5 cells). After 48 hours of transfection, levels of GLP-1 in the supernatant and cell lysates were measured. As is shown in FIG. 8A, transfection with either hybrid transgene resulted in GLP-1 production in the supernatants. As shown in FIG. 8B, similar results were observed in the cell lysate, indicating that the hybrid transgenes were able to successfully produce and secrete GLP-1. The level of total GLP-1 detected in the supernatant of EndoC-BH5 cells (FIG. 8A), which is indicative of glucose-responsive secretion of the transgene, is significantly higher than the level of total GLP-1 detected within the cells (FIG. 8B), suggestive of robust processing of the transgenic glucoregulatory hormone and subsequent nutrient-responsive transgene secretion.

### Example 3—In Vivo Experiments in Type 2 Diabetes Mouse Model

An AAV-based gene therapy candidate was tested in a db/db T2D mouse model to determine its impact on disease progression and severity. Four-week-old db/db male mice were separated into different treatment groups (n=8/group). On day 1, mice were treated with their respect AAV compositions (vehicle control, MIP-eGFP (10e12 VG/animal), MIP-Ex4 (2.5e12 VG/animal), or MIP-Ex4 (10e12 VG/animal)). Note that “Ex4” refers to “Exendin-4,” a glucagon-like peptide 1 (GLP-1) receptor agonist and “MIP” refers to a mouse insulin promoter. On days-1, 8, 15, 22, 29, 36, 43, 50, 57, and 64, mice underwent a 4 hour fasting period and blood glucose was measured. On days-1, 22, 36, 50, and 64, insulin levels were also measured following the fasting period. On day 39, intraperitoneal glucose tolerance tests (IPGTT) were administered and tracked over 120 minutes (0 minutes, 15 minutes, 30 minutes, 60 minutes, and 120 minutes). On day 70, tissue was collected for analysis.

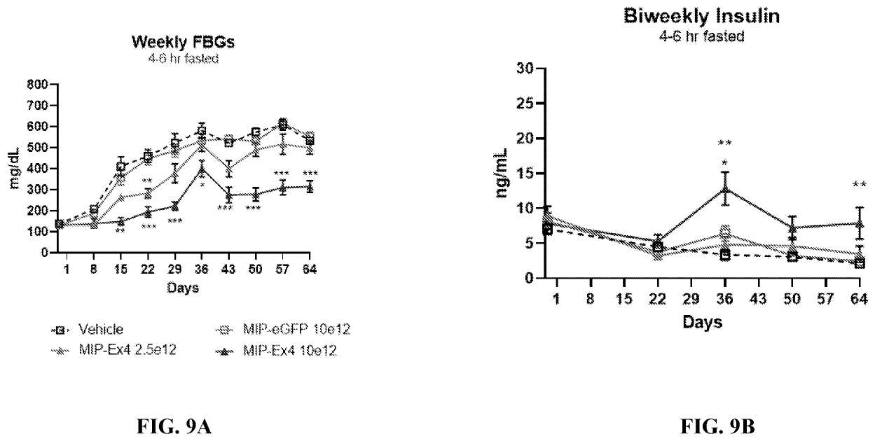

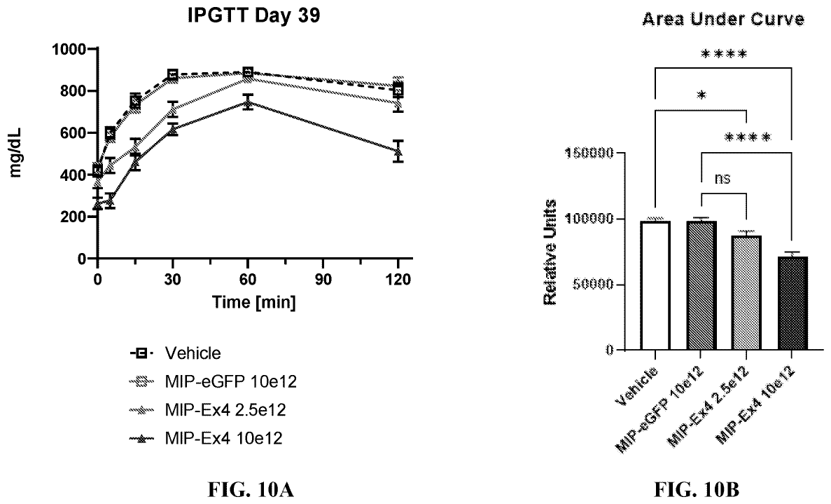

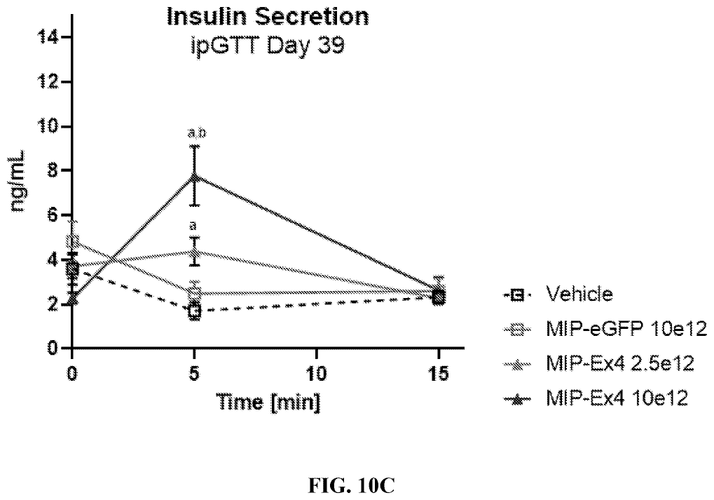

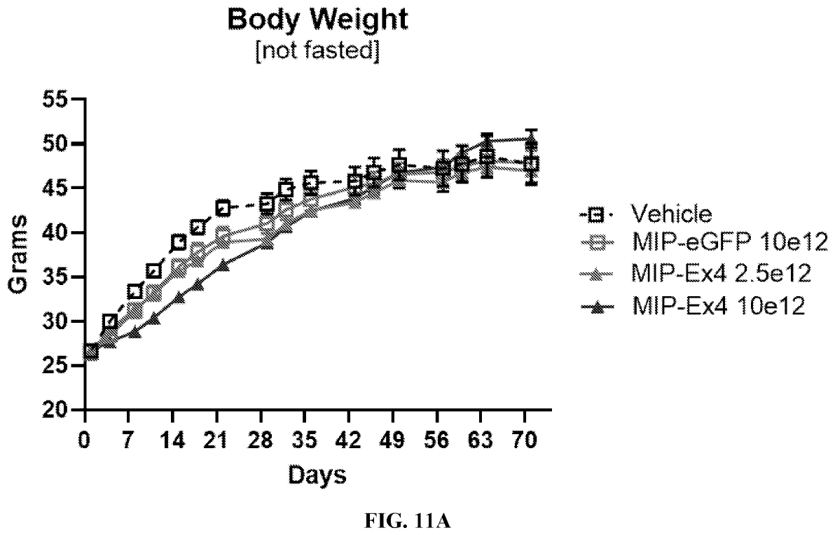

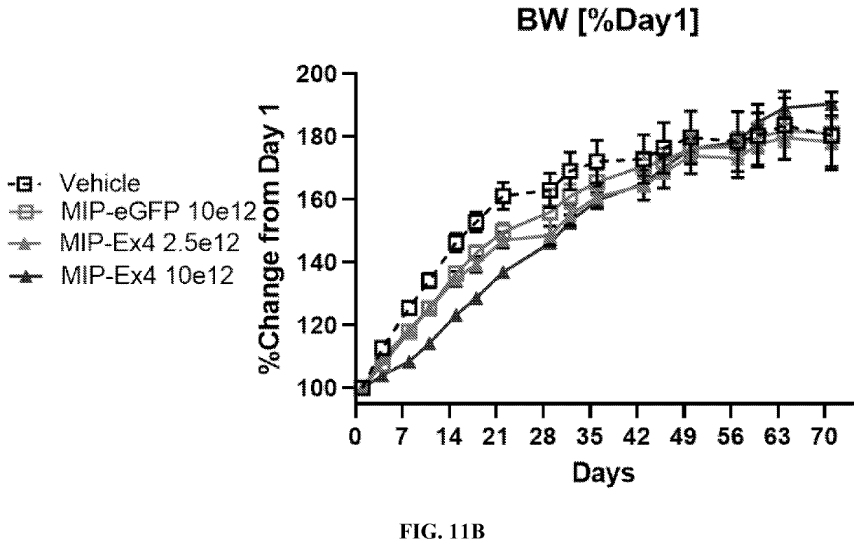

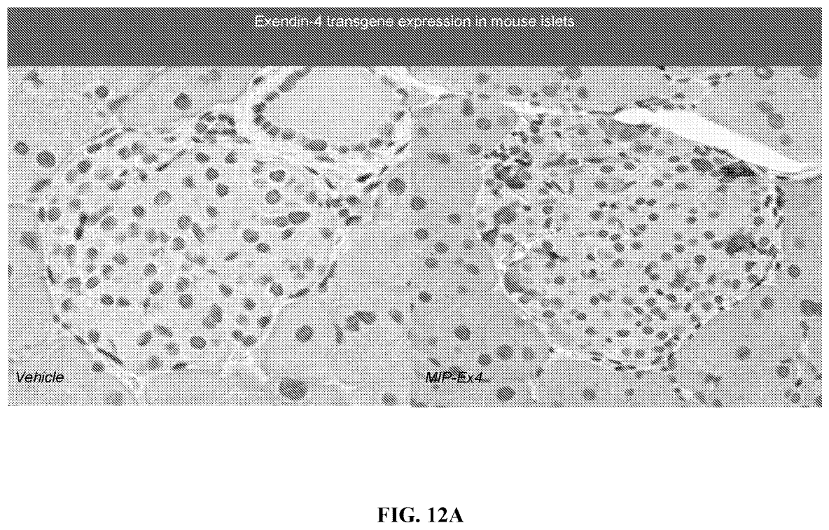

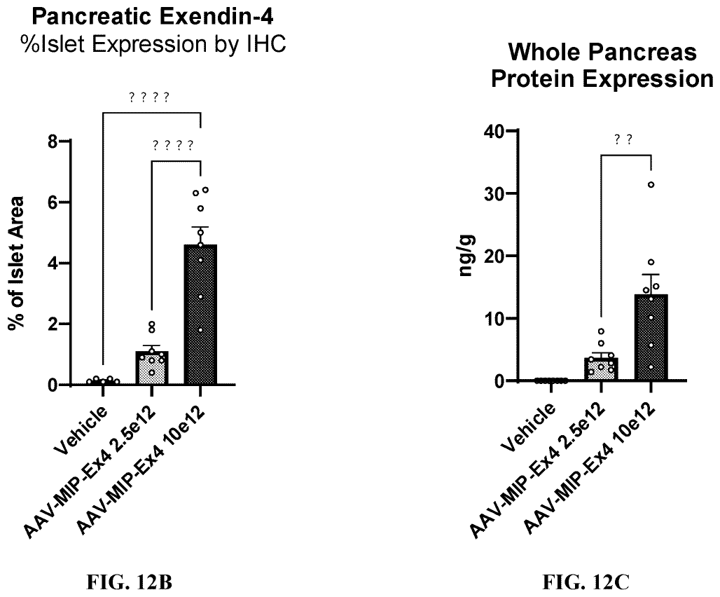

Dose-dependent and durable glycemic control in db/db mice for 10-weeks post-injection was observed. High-dose MIP-Ex4-treated mice exhibited a 59% reduction [Δ 304 mg/dL] in fasting blood glucose (p<0.0001) (FIG. 9A) and a 2.8-fold increase in fasting insulin (p=0.004) (FIG. 9B). A significant improvement in both glucose tolerance (p<0.0001) (FIGS. 10A and 10B) and glucose-stimulated insulin secretion (p<0.05) via intraperitoneal glucose tolerance test (IPGTT) (FIG. 10C) was observed, with no effect on body weight (FIGS. 11A-11B). Immunohistochemical analysis showed that the GLP-1RA protein was expressed in the pancreas (FIG. 12C) and was restricted to the islet cells (FIGS. 12A and 12B).

### Example 4—In Vitro Experiments in Mouse Insulinoma (MIN-6) Cells (Peptide Tyrosine Tyrosine)

DNA expression plasmids were generated to enable transient transfection of different transgenes into a murine pancreatic beta cell model in order to test for each transgene's ability to encode for functional peptide tyrosine tyrosine (PYY) secretion. The transgenes tested are provided in Table 2 and Table 3. Briefly, INS-PYY hybrids (#01-#03) were cloned into an expression plasmid under the control of a human cytomegalovirus (CMV) promoter. The controls included the following groups: no transfection (cells only), empty plasmids, and plasmids expressing an enhanced green fluorescence protein (eGFP) transgene.

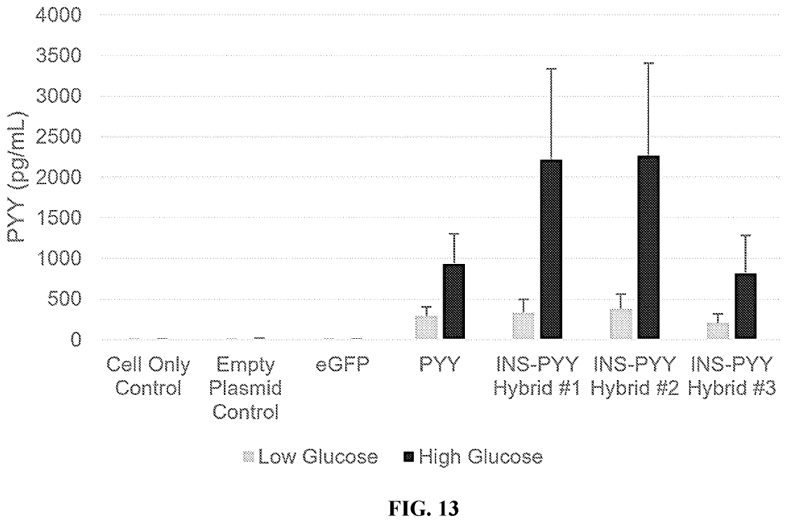

The mouse MIN-6 cell line, a commonly used model of beta cells, was transfected with the expression plasmids described above. Following transfection, cells were exposed to 25 mM glucose plus 0.100 mM 3-isobutyl-1-methylxanthine (IBMX) (high glucose) or 2 mM glucose (low glucose) and then the levels of PYY in the supernatants were measured. The results are shown in FIG. 13 and demonstrate that transfection with multiple INS-PYY hybrid expression plasmids resulted in secreted PYY.

### Example 5—In Vitro Experiments in Mouse Insulinoma (MIN-6) Cells

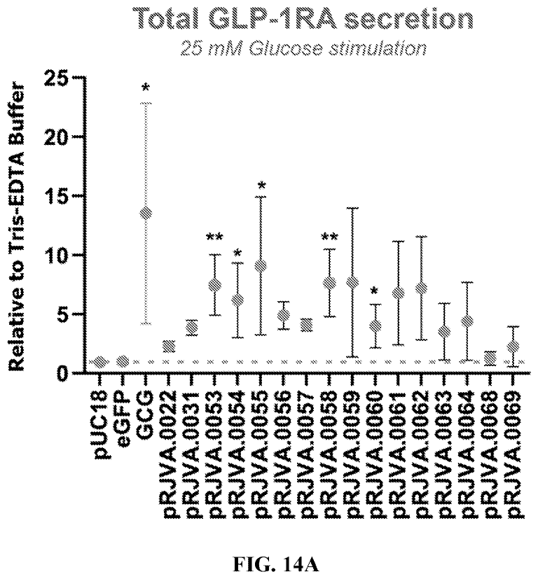

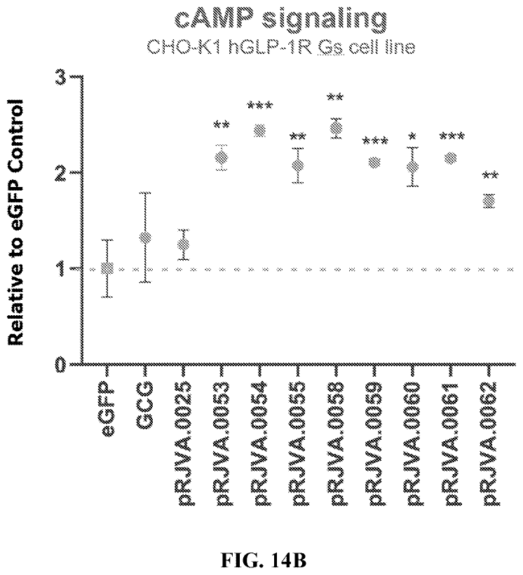

The mouse MIN-6 cell line was transfected with a DNA construct library including plasmids encoding GLP-1RAs. After transfection, cells were exposed to 25 mM glucose and then the levels of GLP-1RA secretion in the cell supernatants were measured. The results are shown in FIG. 14A and demonstrate that transfection multiple GLP-1RA constructs resulted in secreted GLP-1RA relative to the control (Tris-EDTA buffer).

The supernatants from conditions that showed high levels of secreted GLP-1RA following stimulation with glucose were then exposed to the CHO-K1 hGLP-1R Gs cell line to assess the functional activity of each GLP-1RA. Cyclic AMP signaling relative to a GFP control was measured and the results are shown in FIG. 14B. Eight of the nine constructs tested had significantly higher levels of cAMP signaling relative to the control.

### Example 6—Ex Vivo Experiments in Mouse and Human Cells

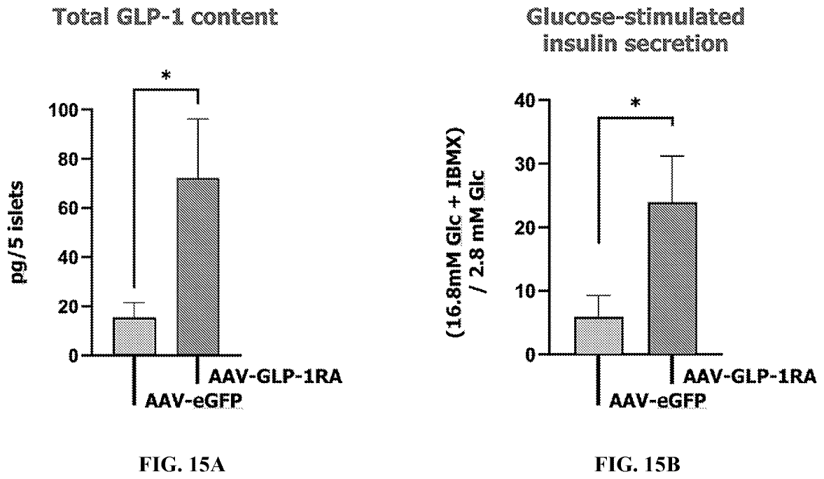

BKS db/db islet cells were isolated and cultured ex vivo and then transduced with an AAV-based GLP-1RA construct comprising Exendin-4 to examine its impact on insulin secretion. Four days after transduction, insulin secretion was measured. In islet cells transduced with the AAV-GLP-1RA construct, there was significantly more GLP-1 (FIG. 15A) and glucose-stimulated insulin secretion (FIG. 15B) compared to the islet cells transduced with AAV-eGFP (control).

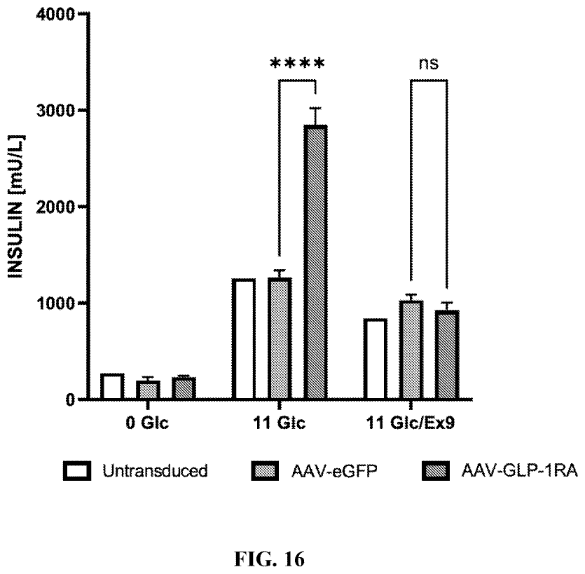

When a human beta cell line, EndoC-BH5, was used, AAV-mediated delivery of GLP-1RA was found to enhance insulin secretion in a GLP-1RA-dependent manner (FIG. 16). In particular, in the presence of glucose, there was a significant difference between the amount of insulin secreted by cells transduced with AAV-GLP-1RA compared to the control (AAV-eGFP). When cells were exposed to both glucose and exendin-9 (Ex9) peptide, a GLP-1RA antagonist, there was no statistically significant difference between groups.

statistically significant difference between groups.

### Example 7—In Vivo Locational Studies

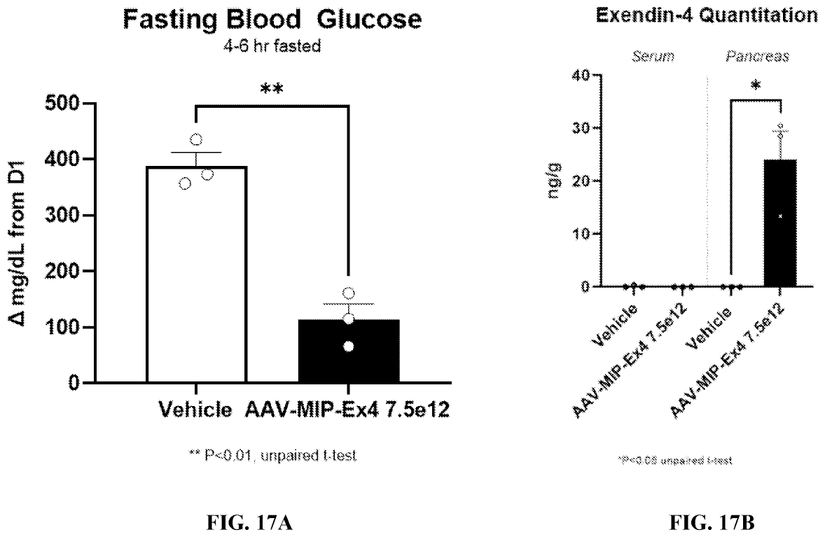

BKS db/db mice were administered an AAV expressing a beta cell-restricted exendin-4 transgene, AAV-MIP-Ex4 (7.5e12 VG/animal), or a vehicle control (n=3/group) Note that “Ex4” refers to “exendin-4,” a glucagon-like peptide 1 (GLP-1) receptor agonist and “MIP” refers to a mouse insulin promoter. Four weeks later, fasting blood glucose was measured (after 4-6 hours of fasting), and the results are shown in FIG. 17A. Significant reductions were observed in mice administered AAV-MIP-Ex4, as compared to the vehicle. In addition, levels of Exendin-4 were measured in the serum and pancreas of each mouse, and the results are shown in FIG. 17B. AAV-based Exendin-4 production was not detectable in the sera of either group of animals; however, high levels of Exendin-4 were detected in the pancreas, as measured via lipid chromatograph mass spectrometry (LCMS), indicated a targeted local delivery of AAV-MIP-Ex4.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. An endoscopic gene therapy method, comprising:
advancing a depositing device comprising a depositing element to a pancreatic deposit site in a patient having a metabolic disease and/or a pancreatic disease; and
delivering an effective amount of a treatment agent through the depositing element into the pancreatic deposit site,
wherein the treatment agent comprises an adeno-associated virus (AAV) vector that comprises an AAV vector genome, wherein the AAV vector genome comprises a polynucleotide comprising a human pancreatic islet beta cell-specific promoter operably linked to a human GLP-1 coding sequence.

2. The method of claim 1, wherein the depositing element comprises a needle positioned on a distal portion of the depositing device.

3. The method of claim 2, wherein the distal portion of the depositing device is delivered into the patient's mouth, and advanced through a gastrointestinal tract wall to a location proximate the patient's pancreas, optionally wherein (a) the depositing device is delivered through a working channel of a gastrointestinal endoscope that has been delivered through the mouth of the patient of (b) the depositing device is delivered alongside a gastrointestinal endoscope that has been delivered through the patient's mouth.

4. The method of claim 1, wherein:
the metabolic disease is selected from the group consisting of: Type 1 diabetes; Type 2 diabetes; nonalcoholic fatty liver disease (NAFLD); nonalcoholic steatohepatitis (NASH); obesity; and combinations thereof; or
the pancreatic disease is selected from the group consisting of: pancreatitis; pancreatic cancer; hyperinsulinism; and combinations thereof.

5. The method of claim 1, wherein the pancreatic deposit site is selected from the group consisting of: intraparenchymal space; anterior pararenal space; intraductal space; intraarterial space of an artery that feeds a portion of the patient's pancreas; and combinations thereof, preferably wherein the pancreatic deposit site is the intraparenchymal space.

6. The method of claim 1, wherein the delivering of a treatment agent comprises a first delivery in which a minimum volume of the treatment agent is delivered into pancreatic parenchyma, and wherein the method further comprises a second delivery of the treatment agent to an additional deposit site proximate a tail tissue of the patient's pancreas.

7. (canceled)

8. The method of claim 1, wherein the depositing device is advanced to the pancreatic deposit site under image guidance, optionally, wherein the image guidance comprises: endoscopic ultrasound guidance; computerized tomography (CT) guidance; and/or magnetic Resonance Imaging (MRI) guidance.

9. The method of claim 1, wherein the pancreatic deposit site comprises locations within 10 cm, 7.5 cm, 5 cm, and/or 3 cm of a portion of the pancreas, and wherein the portion of the patient's pancreas comprises tail, neck, body, head, and/or uncinate process tissue.

10. The method of claim 1, wherein the treatment agent and/or the depositing element is configured to be visualized by an imaging device, and wherein the method further comprises visualizing the treatment agent and/or the depositing element with the imaging device to confirm proper delivery of the treatment agent.

11. The method of claim 1, further comprising delivering an imaging agent through the depositing element and visualizing delivery of the imaging agent with an imaging device to confirm subsequently proper delivery of the treatment agent.

12.-15. (canceled)

16. The method of claim 1, further comprising confirming the depositing element is in a proper location prior to delivering the treatment agent.

17. The method of claim 1, further comprising delivering a permeability-enhancing agent prior to the delivering of the treatment agent and/or simultaneously with the delivering of the treatment agent, optionally wherein the delivering of the permeability-enhancing agent is performed locally and/or intravenously, optionally wherein the permeability-enhancing agent comprises an agent selected from the group consisting of: hyaluronidase; collagenase; losartan; and combinations thereof, and optionally wherein the treatment agent comprises a coformulation of the treatment agent and the permeability-enhancing agent.

18. (canceled)

19. The method of claim 1, further comprising delivering a dissemination-blocking material that is configured to prevent undesired dissemination of the treatment agent to non-target locations, optionally wherein the dissemination-blocking material comprises a viscous substance and/or a polymer.

20. The method of claim 1, further comprising positioning a blocking element in the patient, wherein the blocking element is configured to prevent undesired dissemination of the treatment agent to non-target locations.

21.-22. (canceled)

23. The method of claim 1, wherein the patient has a metabolic disease.

24. The method of claim 23, wherein the metabolic disease is selected from: Type 1 diabetes; Type 2 diabetes; nonalcoholic fatty liver disease (NAFLD); nonalcoholic steatohepatitis (NASH); and obesity.

25. The method of claim 1, wherein the patient has a pancreatic disease.

26. The method of claim 25, wherein the pancreatic disease is selected from pancreatitis; pancreatic cancer; and hyperinsulinism.

27. An endoscopic gene therapy method, comprising:
advancing a depositing device comprising a depositing element to a pancreatic deposit site in a patient having a metabolic disease and/or a pancreatic disease; and
delivering an effective amount of a treatment agent through the depositing element into the pancreatic deposit site,
wherein the treatment agent comprises an adeno-associated virus (AAV) vector that comprises an AAV vector genome, wherein the AAV vector genome comprises a polynucleotide comprising a human pancreatic islet beta cell-specific promoter operably linked to a glucose-dependent insulinotropic peptide (GIP).

28. An endoscopic gene therapy method, comprising:
advancing a depositing device comprising a depositing element to a pancreatic deposit site in a patient having a metabolic disease and/or a pancreatic disease; and
delivering an effective amount of a treatment agent through the depositing element into the pancreatic deposit site,
wherein the treatment agent comprises an adeno-associated virus (AAV) vector that comprises an AAV vector genome, wherein the AAV vector genome comprises a polynucleotide comprising a human pancreatic islet beta cell-specific promoter operably linked to a peptide tyrosine tyrosine (PYY) coding sequence.


SEQUENCE LISTING (94 sequences)

SEQ ID NO: 1 - DNA, 543 bp, synthetic construct
  source 1..543: mol_type = other DNA
atgaaaagcatttactttgtggctgggttatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataa

SEQ ID NO: 2 - DNA, 87 bp, synthetic construct
  source 1..87: mol_type = other DNA
cattcacagggcacattcaccagtgactacagcaagtatctggactccaggcgtgcccaagattttgtgcagtggttgatgaatacc

SEQ ID NO: 3 - DNA, 111 bp, synthetic construct
  source 1..111: mol_type = other DNA
cacgatgaatttgagagacatgctgaagggacctttaccagtgatgtaagttcttatttggaaggccaagctgccaaggaattcattgcttggctggtgaaaggccgagga

SEQ ID NO: 4 - DNA, 111 bp, synthetic construct
  source 1..111: mol_type = other DNA
cattcacagggcacattcaccagtgactacagcaagtatctggactccaggcgtgcccaagattttgtgcagtggttgatgaataccaagaggaacaggaataacattgcc

SEQ ID NO: 5 - DNA, 207 bp, synthetic construct
  source 1..207: mol_type = other DNA
cgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgcc

SEQ ID NO: 6 - DNA, 90 bp, synthetic construct
  source 1..90: mol_type = other DNA
cgttcccttcaagacacagaggagaaatccagatcattctcagcttcccaggcagacccactcagtgatcctgatcagatgaacgaggac

SEQ ID NO: 7 - DNA, 261 bp, synthetic construct
  source 1..261: mol_type = other DNA
cacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgac

SEQ ID NO: 8 - DNA, 18 bp, synthetic construct
  source 1..18: mol_type = other DNA
aacaggaataacattgcc

SEQ ID NO: 9 - DNA, 39 bp, synthetic construct
  source 1..39: mol_type = other DNA
gatttcccagaagaggtcgccattgttgaagaacttggc

SEQ ID NO: 10 - DNA, 99 bp, synthetic construct
  source 1..99: mol_type = other DNA
catgctgatggttctttctctgatgagatgaacaccattcttgataatcttgccgccagggactttataaactggttgattcagaccaaaatcactgac

SEQ ID NO: 11 - DNA, 462 bp, synthetic construct
  source 1..462: mol_type = other DNA
atggtggccacgaagacctttgctctgctgctgctgtccctgttcctggcagtgggactaggagagaagaaagagggtcacttcagcgctctcccctccctgcctgttggatctcatgctaaggtgagcagccctcaacctcgaggccccaggtacgcggaagggactttcatcagtgactacagtattgccatggacaagattcaccaacaagactttgtgaactggctgctggcccaaaaggggaagaagaatgactggaaacacaacatcacccagagggaggctcgggcgctggagctggccagtcaagctaataggaaggaggaggaggcagtggagccacagagctccccagccaagaaccccagcgatgaagatttgctgcgggacttgctgattcaagagctgttggcctgcttgctggatcagacaaacctctgcaggctcaggtctcggtga

SEQ ID NO: 12 - DNA, 294 bp, synthetic construct
  source 1..294: mol_type = other DNA
atggtgttcgtgcgcaggccgtggcccgccttgaccacagtgcttctggccctgctcgtctgcctaggggcgctggtcgacgcctaccccatcaaacccgaggctccccgcgaagacgcctcgccggaggagctgaaccgctactacgcctccctgcgccactacctcaacctggtcacccggcagcggtatgggaaaagagacggcccggacacgcttctttccaaaacgttcttccccgacggcgaggaccgccccgtcaggtcgcggtcggagggcccagacctgtggtga

SEQ ID NO: 13 - DNA, 348 bp, synthetic construct
  source 1..348: mol_type = other DNA
atgaacagcggcgtgtgcctgtgcgtgctgatggcggtactggcggctggcgccctgacgcagccggtgcctcccgcagatcccgcgggctccgggctgcagcgggcagaggaggcgccccgtaggcagctgagggtatcgcagagaacggatggcgagtcccgagcgcacctgggcgccctgctggcaagatacatccagcaggcccggaaagctccttctggacgaatgtccatcgttaagaacctgcagaacctggaccccagccacaggataagtgaccgggactacatgggctggatggattttggccgtcgcagtgccgaggagtatgagtacccctcctag

SEQ ID NO: 14 - DNA, 351 bp, synthetic construct
  source 1..351: mol_type = other DNA
atgctgtcctgccgcctccagtgcgcgctggctgcgctgtccatcgtcctggccctgggctgtgtcaccggcgctccctcggaccccagactccgtcagtttctgcagaagtccctggctgctgccgcggggaagcaggaactggccaagtacttcttggcagagctgctgtctgaacccaaccagacggagaatgatgccctggaacctgaagatctgtcccaggctgctgagcaggatgaaatgaggcttgagctgcagagatctgctaactcaaacccggctatggcaccccgagaacgcaaagctggctgcaagaatttcttctggaagactttcacatcctgttag

SEQ ID NO: 15 - DNA, 351 bp, synthetic construct
  source 1..351: mol_type = other DNA
atgccctccccagggaccgtctgcagcctcctgctcctcggcatgctctggctggacttggccatggcaggctccagcttcctgagccctgaacaccagagagtccagagaaaggagtcgaagaagccaccagccaagctgcagccccgagctctagcaggctggctccgcccggaagatggaggtcaagcagaaggggcagaggatgaactggaagtccggttcaacgccccctttgatgttggaatcaagctgtcaggggttcagtaccagcagcacagccaggccctggggaagtttcttcaggacatcctctgggaagaggccaaagaggccccagccgacaagtga

SEQ ID NO: 16 - DNA, 333 bp, synthetic construct
  source 1..333: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 17 - DNA, 270 bp, synthetic construct
  source 1..270: mol_type = other DNA
atgggcatcctgaagctgcaagtatttctcattgtgctctctgttgcattgaaccatctgaaagctacacccattgaaagtcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttag

SEQ ID NO: 18 - DNA, 504 bp, synthetic construct
  source 1..504: mol_type = other DNA
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 19 - DNA, 954 bp, synthetic construct
  source 1..954: mol_type = other DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactga

SEQ ID NO: 20 - DNA, 462 bp, synthetic construct
  source 1..462: mol_type = other DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 21 - DNA, 513 bp, synthetic construct
  source 1..513: mol_type = other DNA
atggacaccagaaataaggcccagctccttgtgctcctgactcttctcagtgtgctcttctcacagacttcggcatggcctctttacagggcaccttctgctctcaggttgggtgacagaataccctttgagggagcaaatgaacctgatcaagtttcattaaaagaagacattgacatgttgcaaaatgcattagctgaaaatgacacaccctattatgatgtatccagaaatgccaggcatgctgatggagttttcaccagtgacttcagtaaactcttgggtcaactttctgccaaaaagtaccttgagtctcttatgggaaaacgtgttagcagtaacatctcagaagaccctgtaccagtcaaacgtcactcagatgcagtcttcactgacaactatacccgccttagaaaacaaatggctgtaaagaaatatttgaactcaattctgaatggaaagaggagcagtgagggagaatctcccgactttccagaagagttagaaaaatga

SEQ ID NO: 22 - DNA, 654 bp, synthetic construct
  source 1..654: mol_type = other DNA
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 23 - DNA, 93 bp, synthetic construct
  source 1..93: mol_type = other DNA
catgctgaagggacctttaccagtgatgtaagttcttatttggaaggccaagctgccaaggaattcattgcttggctggtgaaaggccgagga

SEQ ID NO: 24 - DNA, 192 bp, synthetic construct
  source 1..192: mol_type = other DNA
aagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcaga

SEQ ID NO: 25 - DNA, 123 bp, synthetic construct
  source 1..123: mol_type = other DNA
aaacgtcacgatgaatttgagagacatgctgaagggacctttaccagtgatgtaagttcttatttggaaggccaagctgccaaggaattcattgcttggctggtgaaaggccgaggaaggcga

SEQ ID NO: 26 - DNA, 147 bp, synthetic construct
  source 1..147: mol_type = other DNA
aagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcga

SEQ ID NO: 27 - DNA, 192 bp, synthetic construct; identical to SEQ ID NO: 24
  source 1..192: mol_type = other DNA

SEQ ID NO: 28 - DNA, 123 bp, synthetic construct; identical to SEQ ID NO: 25
  source 1..123: mol_type = other DNA

SEQ ID NO: 29 - DNA, 168 bp, synthetic construct
  source 1..168: mol_type = other DNA
aaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcaga

SEQ ID NO: 30 - DNA, 156 bp, synthetic construct
  source 1..156: mol_type = other DNA
aagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttccca

SEQ ID NO: 31 - protein, 180 aa, synthetic construct
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 32 - protein, 29 aa, synthetic construct
HSQGTFTSDYSKYLDSRRAQDFVQWLMNT

SEQ ID NO: 33 - protein, 37 aa, synthetic construct
HDEFERHAEGTFTSDVSSYLEGQAAKEFIAWLVKGRG

SEQ ID NO: 34 - protein, 37 aa, synthetic construct
HSQGTFTSDYSKYLDSRRAQDFVQWLMNTKRNRNNIA

SEQ ID NO: 35 - protein, 69 aa, synthetic construct
RSLQDTEEKSRSFSASQADPLSDPDQMNEDKRHSQGTFTSDYSKYLDSRRAQDFVQWLMNTKRNRNNIA

SEQ ID NO: 36 - protein, 30 aa, synthetic construct
RSLQDTEEKSRSFSASQADPLSDPDQMNED

SEQ ID NO: 37 - protein, 87 aa, synthetic construct
HDEFERHAEGTFTSDVSSYLEGQAAKEFIAWLVKGRGRRDFPEEVAIVEELGRRHADGSFSDEMNTILDNLAARDFINWLIQTKITD

SEQ ID NO: 38 - protein, 6 aa, synthetic construct
NRNNIA

SEQ ID NO: 39 - protein, 13 aa, synthetic construct
DFPEEVAIVEELG

SEQ ID NO: 40 - protein, 33 aa, synthetic construct
HADGSFSDEMNTILDNLAARDFINWLIQTKITD

SEQ ID NO: 41 - protein, 153 aa, synthetic construct
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 42 - protein, 97 aa, synthetic construct
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 43 - protein, 115 aa, synthetic construct
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 44 - protein, 116 aa, synthetic construct
MLSCRLQCALAALSIVLALGCVTGAPSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 45 - protein, 116 aa, synthetic construct
MPSPGTVCSLLLLGMLWLDLAMAGSSFLSPEHQRVQRKESKKPPAKLQPRALAGWLRPEDGGQAEGAEDELEVRFNAPFDVGIKLSGVQYQQHSQALGKFLQDILWEEAKEAPADK

SEQ ID NO: 46 - protein, 110 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 47 - protein, 89 aa, synthetic construct
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 48 - protein, 167 aa, synthetic construct
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 49 - protein, 317 aa, synthetic construct
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 50 - protein, 153 aa, synthetic construct
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 51 - protein, 170 aa, synthetic construct
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 52 - protein, 217 aa, synthetic construct
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 53 - protein, 31 aa, synthetic construct
HAEGTFTSDVSSYLEGQAAKEFIAWLVKGRG

SEQ ID NO: 54 - protein, 49 aa, synthetic construct
KRNRNNIAKRHDEFERHAEGTFTSDVSSYLEGQAAKEFIAWLVKGRGRR

SEQ ID NO: 55 - protein, 64 aa, synthetic construct
KRNRNNIAKRHDEFERHAEGTFTSDVSSYLEGQAAKEFIAWLVKGRGRRDFPEEVAIVEELGRR

SEQ ID NO: 56 - protein, 41 aa, synthetic construct
KRHDEFERHAEGTFTSDVSSYLEGQAAKEFIAWLVKGRGRR

SEQ ID NO: 57 - protein, 56 aa, synthetic construct
KRHDEFERHAEGTFTSDVSSYLEGQAAKEFIAWLVKGRGRRDFPEEVAIVEELGRR

SEQ ID NO: 58 - protein, 52 aa, synthetic construct
KRNRNNIAKRHDEFERHAEGTFTSDVSSYLEGQAAKEFIAWLVKGRGRRDFP

SEQ ID NO: 59 - DNA, 489 bp, synthetic construct
  source 1..489: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccaaagcgtggcattgtggaacaatgctgtaccagcatctgctccctctaccagctggagaactactgcaactag

SEQ ID NO: 60 - DNA, 525 bp, synthetic construct
  source 1..525: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagaaagcgtggcattgtggaacaatgctgtaccagcatctgctccctctaccagctggagaactactgcaactag

SEQ ID NO: 61 - DNA, 525 bp, synthetic construct
  source 1..525: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagaccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 62 - DNA, 525 bp, synthetic construct
  source 1..525: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggcaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacctggtgcaggcagcctgcagcccttggccctggaggggtccctgcagaagcgtggcattgtggaacaatgctgtaccagcatctgctccctctaccagctggagaactactgcaactag

SEQ ID NO: 63 - DNA, 501 bp, synthetic construct
  source 1..501: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagaaagcgtggcattgtggaacaatgctgtaccagcatctgctccctctaccagctggagaactactgcaactag

SEQ ID NO: 64 - DNA, 480 bp, synthetic construct
  source 1..480: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgaaagcgtggcattgtggaacaatgctgtaccagcatctgctccctctaccagctggagaactactgcaactag

SEQ ID NO: 65 - DNA, 456 bp, synthetic construct
  source 1..456: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagaccaaacgtcacgatgaatttgagagacatgctgaagggacctttaccagtgatgtaagttcttatttggaaggccaagctgccaaggaattcattgcttggctggtgaaaggccgaggaaggcgacgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 66 - DNA, 456 bp, synthetic construct
  source 1..456: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggcaaacgtcacgatgaatttgagagacatgctgaagggacctttaccagtgatgtaagttcttatttggaaggccaagctgccaaggaattcattgcttggctggtgaaaggccgaggaaggcgacctggtgcaggcagcctgcagcccttggccctggaggggtccctgcagaagcgtggcattgtggaacaatgctgtaccagcatctgctccctctaccagctggagaactactgcaactag

SEQ ID NO: 67 - DNA, 456 bp, synthetic construct
  source 1..456: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaaacgtcacgatgaatttgagagacatgctgaagggacctttaccagtgatgtaagttcttatttggaaggccaagctgccaaggaattcattgcttggctggtgaaaggccgaggaaggcgaaagcgtggcattgtggaacaatgctgtaccagcatctgctccctctaccagctggagaactactgcaactag

SEQ ID NO: 68 - DNA, 525 bp, synthetic construct
  source 1..525: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaacaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagatag

SEQ ID NO: 69 - DNA, 456 bp, synthetic construct
  source 1..456: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaacaaacgtcacgatgaatttgagagacatgctgaagggacctttaccagtgatgtaagttcttatttggaaggccaagctgccaaggaattcattgcttggctggtgaaaggccgaggaaggcgatag

SEQ ID NO: 70 - DNA, 525 bp, synthetic construct
  source 1..525: mol_type = other DNA
atggccctgtggatgcgcctcctgcccctgctggcgctgctggccctctggggacctgacccagccgcagccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagatttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 71 - DNA, 456 bp, synthetic construct
  source 1..456: mol_type = other DNA
atggccctgtggatgcgcctcctgcccctgctggcgctgctggccctctggggacctgacccagccgcagccaaacgtcacgatgaatttgagagacatgctgaagggacctttaccagtgatgtaagttcttatttggaaggccaagctgccaaggaattcattgcttggctggtgaaaggccgaggaaggcgatttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 72 - protein, 162 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRNRNNIAKRHDEFERHAEGTFTSDVSSYLEGQAAKEFIAWLVKGRGRRDFPKRGIVEQCCTSICSLYQLENYCN

SEQ ID NO: 73 - protein, 174 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRNRNNIAKRHDEFERHAEGTFTSDVSSYLEGQAAKEFIAWLVKGRGRRDFPEEVAIVEELGRRKRGIVEQCCTSICSLYQLENYCN

SEQ ID NO: 74 - protein, 174 aa, synthetic construct
MALWMRLLPLLALLALWGPDPAAAFVNQHLCGSHLVEALYLVCGERGFFYTPKTKRNRNNIAKRHDEFERHAEGTFTSDVSSYLEGQAAKEFIAWLVKGRGRRDFPEEVAIVEELGRRRREAEDLQVGQVELGGGPGAGSLQPLALEGSLQKRGIVEQCCTSICSLYQLENYCN

SEQ ID NO: 75 - protein, 174 aa, synthetic construct
MALWMRLLPLLALLALWGPDPAAAFVNQHLCGSHLVEALYLVCGERGFFYTPKTRREAEDLQVGQVELGGGKRNRNNIAKRHDEFERHAEGTFTSDVSSYLEGQAAKEFIAWLVKGRGRRDFPEEVAIVEELGRRPGAGSLQPLALEGSLQKRGIVEQCCTSICSLYQLENYCN

SEQ ID NO: 76 - protein, 166 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRHDEFERHAEGTFTSDVSSYLEGQAAKEFIAWLVKGRGRRDFPEEVAIVEELGRRKRGIVEQCCTSICSLYQLENYCN

SEQ ID NO: 77 - protein, 159 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRNRNNIAKRHDEFERHAEGTFTSDVSSYLEGQAAKEFIAWLVKGRGRRKRGIVEQCCTSICSLYQLENYCN

SEQ ID NO: 78 - protein, 151 aa, synthetic construct
MALWMRLLPLLALLALWGPDPAAAFVNQHLCGSHLVEALYLVCGERGFFYTPKTKRHDEFERHAEGTFTSDVSSYLEGQAAKEFIAWLVKGRGRRRREAEDLQVGQVELGGGPGAGSLQPLALEGSLQKRGIVEQCCTSICSLYQLENYCN

SEQ ID NO: 79 - protein, 151 aa, synthetic construct
MALWMRLLPLLALLALWGPDPAAAFVNQHLCGSHLVEALYLVCGERGFFYTPKTRREAEDLQVGQVELGGGKRHDEFERHAEGTFTSDVSSYLEGQAAKEFIAWLVKGRGRRPGAGSLQPLALEGSLQKRGIVEQCCTSICSLYQLENYCN

SEQ ID NO: 80 - protein, 151 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRHDEFERHAEGTFTSDVSSYLEGQAAKEFIAWLVKGRGRRKRGIVEQCCTSICSLYQLENYCN

SEQ ID NO: 81 - protein, 174 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNKRNRNNIAKRHDEFERHAEGTFTSDVSSYLEGQAAKEFIAWLVKGRGRRDFPEEVAIVEELGRR

SEQ ID NO: 82 - protein, 151 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNKRHDEFERHAEGTFTSDVSSYLEGQAAKEFIAWLVKGRGRR

SEQ ID NO: 83 - protein, 174 aa, synthetic construct
MALWMRLLPLLALLALWGPDPAAAKRNRNNIAKRHDEFERHAEGTFTSDVSSYLEGQAAKEFIAWLVKGRGRRDFPEEVAIVEELGRRFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 84 - protein, 151 aa, synthetic construct
MALWMRLLPLLALLALWGPDPAAAKRHDEFERHAEGTFTSDVSSYLEGQAAKEFIAWLVKGRGRRFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 85 - DNA, 450 bp, synthetic construct
  source 1..450: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccggtaccccatcaaacccgaggctccccgcgaagacgcctcgccggaggagctgaaccgctactacgcctccctgcgccactacctcaacctggtcacccggcagcggtatgggaaaagagaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 86 - DNA, 450 bp, synthetic construct
  source 1..450: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgttaccccatcaaacccgaggctccccgcgaagacgcctcgccggaggagctgaaccgctactacgcctccctgcgccactacctcaacctggtcacccggcagcggtatgggaaaagaggcattgtggaacaatgctgtaccagcatctgctccctctaccagctggagaactactgcaactag

SEQ ID NO: 87 - DNA, 456 bp, synthetic construct
  source 1..456: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaacaagcgttaccccatcaaacccgaggctccccgcgaagacgcctcgccggaggagctgaaccgctactacgcctccctgcgccactacctcaacctggtcacccggcagcggtatgggaaaagatag

SEQ ID NO: 88 - protein, 149 aa, synthetic construct
MALWMRLLPLLALLALWGPDPAAAFVNQHLCGSHLVEALYLVCGERGFFYTPKTRRYPIKPEAPREDASPEELNRYYASLRHYLNLVTRQRYGKREAEDLQVGQVELGGGPGAGSLQPLALEGSLQKRGIVEQCCTSICSLYQLENYCN

SEQ ID NO: 89 - protein, 149 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRYPIKPEAPREDASPEELNRYYASLRHYLNLVTRQRYGKRGIVEQCCTSICSLYQLENYCN

SEQ ID NO: 90 - protein, 151 aa, synthetic construct
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCNKRYPIKPEAPREDASPEELNRYYASLRHYLNLVTRQRYGKR

SEQ ID NO: 91 - DNA, 117 bp, synthetic construct
  source 1..117: mol_type = other DNA
taccccatcaaacccgaggctccccgcgaagacgcctcgccggaggagctgaaccgctactacgcctccctgcgccactacctcaacctggtcacccggcagcggtatgggaaaaga

SEQ ID NO: 92 - DNA, 108 bp, synthetic construct
  source 1..108: mol_type = other DNA
taccccatcaaacccgaggctccccgcgaagacgcctcgccggaggagctgaaccgctactacgcctccctgcgccactacctcaacctggtcacccggcagcggtat

SEQ ID NO: 93 - protein, 39 aa, synthetic construct
YPIKPEAPREDASPEELNRYYASLRHYLNLVTRQRYGKR

SEQ ID NO: 94 - protein, 36 aa, synthetic construct
YPIKPEAPREDASPEELNRYYASLRHYLNLVTRQRY